(19) United States
(12) Patent Application Publication
(10) Pub. No.: US 2024/0391902 A1
(43) Pub. Date: Nov. 28, 2024

(54) Crystalline Form of Compound

(71) Applicant: Shenzhen Forward Pharmaceuticals Co., Limited, Shenzhen (CN)

(72) Inventors: Chenggang Zhu, Shenzhen (CN); Xuan Yang, Shenzhen (CN); Chaochun Zhang, Shenzhen (CN); Liangliang Xu, Shenzhen (CN)

(21) Appl. No.: 18/011,741

(22) PCT Filed: Jun. 23, 2021
(86) PCT No.: PCT/CN2021/101779

§ 371 Date: Dec. 20, 2022

(30) Foreign Application Priority Data
Jun. 23, 2020 (CN) 202010579560.2
May 22, 2021 (CN) 202110563762.2

(51) Int. Cl. C07D 403/04; A61K 31/53; C01B 7/01; C07C 57/145; C07C 309/04

(52) U.S. Cl. CPC C07D 403/04; A61K 31/53; C01B 7/01; C07C 57/145; C07C 309/04

(57) ABSTRACT

The present application relates to the crystalline form of the mesylate salt, the crystalline form of the hydrochloride salt and the crystalline form of the maleate salt of a compound of formula (I):

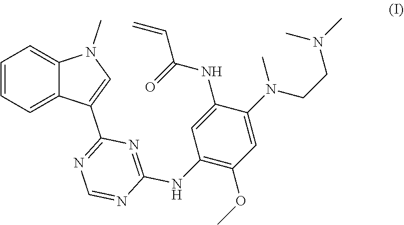 [SMILES: C=CC(=O)Nc1cc(Nc2ncnc(-c3cn(C)c4ccccc34)n2)c(OC)cc1N(C)CCN(C)C]

The present application also relates to a method for treating non-small cell lung cancer (NSCLC) with EGFR exon 20 insertion mutation using the crystalline forms. The present application also relates to synthetic routes for the compound of formula (I).

### TECHNICAL FIELD

The present application relates to the crystalline form of the mesylate salt, the crystalline form of the hydrochloride salt and the crystalline form of the maleate salt of a compound of formula (I):

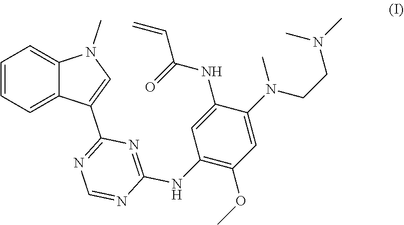 [SMILES: C=CC(=O)Nc1cc(Nc2ncnc(-c3cn(C)c4ccccc34)n2)c(OC)cc1N(C)CCN(C)C]

The present application also relates to a method for treating non-small cell lung cancer (NSCLC) with EGFR exon 20 insertion mutation using the crystalline forms. The present application also relates to synthetic routes for the compound of formula (I).

### BACKGROUND ART

In the field of anticancer drugs, EGFR is known as a member of the transmembrane protein tyrosine kinase of the erbB receptor family.

Homodimerization and/or heterodimerization of the erbB receptor leads to phosphorylation of certain tyrosine residues in the intracellular domain and activates multiple intracellular signaling pathways involved in cell proliferation and survival. Dysregulation of erbB family signaling can lead to cell proliferation, invasion, metastasis, and angiogenesis, and has been reported in cancers such as lung, head and neck, and breast cancer. Therefore, as targets for anticancer drug development, many drugs targeting EGFR have been clinically applied.

Chinese patent CN105461695B discloses a compound of formula (I), which have multiple basic centers. The inhibitory activity of the compound against EGFR activating mutation (such as exon 19 deletion activating mutation, L858R activating mutation, T790M drug resistance mutation and exon 20 insertion mutation) is significantly higher than the inhibitory activity against wild-type EGFR (WT EGFR), and therefore, the compound has higher selectivity and safety, and lower toxic and side effects.

Salt formation studies are usually carried out on organic basic compounds having activity. However, those skilled in the art cannot predict with which acids a specific organic basic compound can form stable salts, or whether a specific organic basic compound or its acid addition salt is more suitable for further drug development, let alone which salt formed has better chemical stability, physical stability or solubility, and which salt has all these better properties. In particular, if an organic basic compound has multiple basic centers, it is impossible for those skilled in the art to predict whether its salts formed with a specific acid in various equivalent ratios have the same properties or different properties, let alone which equivalent ratio of the organic basic compound to the acid is more suitable for further drug development.

After the salt formation study of the organic basic compounds having activity, the crystallization study of the screened salt is usually further carried out, because in addition to the properties brought by the salt formation, the crystallization is likely to provide the salt with additional properties that are more suitable for further drug development, such as better stability, processability, and druggability. However, although there is general guidance on crystallization in the prior art, such general guidance does not enable a person skilled in the art to reasonably determine whether a specific salt formed from a specific organic basic compound and a specific acid can form a particular crystalline form, let alone whether such a particular crystalline form has additional properties that are more suitable for further drug development.

### SUMMARY OF THE INVENTION

The present application seeks to find salts of the compound of formula (I) suitable for further drug development. In particular, the present application seeks to find salts with properties suitable for further drug development, including a reasonable salt-forming equivalent ratio, better chemical stability, better physical stability and/or better solubility. The present application is further intended to seek crystalline forms of the salt of the compound of formula (I) suitable for further drug development based on the salts of the compound of formula (I) screened out.

In one aspect according to the invention, the present application relates to a salt formed from the compound of formula (I) with an acid:

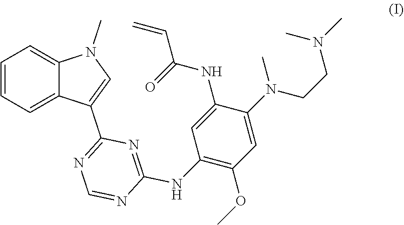 [SMILES: C=CC(=O)Nc1cc(Nc2ncnc(-c3cn(C)c4ccccc34)n2)c(OC)cc1N(C)CCN(C)C]

wherein the acid is selected from hydrochloric acid, methanesulfonic acid, benzenesulfonic acid, ethanesulfonic acid, maleic acid, hydrobromic acid, citric acid, L-tartaric acid, and p-toluenesulfonic acid.

In another aspect according to the invention, the present application relates to a crystalline form of the mesylate salt of the compound of formula (I), a crystalline form of the hydrochloride salt of the compound of formula (I) and a crystalline form of the maleate salt of the compound of formula (I).

Preferably, the crystalline form of the mesylate salt of the compound of formula (I) is crystalline form II-A, which is characterized in that the crystalline form II-A at least has an X-ray powder diffraction pattern having characteristic peaks expressed in 2θ° of 15.12°±0.2°, 22.28°±0.2° and 25.82°±0.2°.

Preferably, the crystalline form of the hydrochloride salt of the compound of formula (I) is crystalline form III-A, which is characterized in that the crystalline form III-A at least has an X-ray powder diffraction pattern having characteristic peaks expressed in 2θ° of 24.85°±0.2°, 14.08°±0.2° and 14.45°±0.2°.

Preferably, the crystalline form of the maleate salt of the compound of formula (I) is crystalline form IV-C, which is characterized in that the crystalline form IV-C at least has an X-ray powder diffraction pattern having characteristic peaks expressed in 2θ° of 20.71°±0.2°, 25.76°±0.2° and 17.00°±0.2°.

In another aspect according to the invention, the present application relates to a pharmaceutical composition comprising the salt or crystalline form according to the invention, which comprises the salt or crystalline form according to the invention and a pharmaceutically acceptable carrier.

In still another aspect according to the invention, the present application relates to a method of treating non-small cell lung cancer (NSCLC) with EGFR exon 20 insertion mutation, comprising administering the salt or crystalline form according to the invention to a patient.

In a further aspect according to the invention, the present application relates to the use of the salt or crystalline form according to the invention in the preparation of a medicament for the treatment of non-small cell lung cancer (NSCLC) with EGFR exon 20 insertion mutation.

### BRIEF DESCRIPTION OF THE DRAWINGS

The accompanying drawings are used for a better understanding of the present application, and do not constitute a limitation to the present application.

FIG. 1: XRPD pattern of the amorphous free base (Sample No. Y11526-45-RV-FWD1509-AF-SU12).

FIG. 2: DSC profile of the amorphous free base (Sample No. Y11526-45-RV-FWD1509-AF-SU12).

FIG. 3: TGA profile of the amorphous free base (Sample No. Y11526-45-RV-FWD1509-AF-SU12).

FIG. 4: ¹H-NMR spectrum of the amorphous free base (Sample No. Y11526-45-RV-FWD1509-AF-SU12).

FIG. 5: PLM photograph of the amorphous free base (Sample No. Y11526-45-RV-FWD1509-AF-SU12).

FIG. 6: XRPD pattern of the amorphous 1 eq. (equivalent) hydrochloride salt (Sample No. Y11526-42-SU11-methanol-dichloromethane).

FIG. 7: DSC profile of the amorphous 1 eq. hydrochloride salt (Sample No. Y11526-42-SU11-methanol-dichloromethane).

FIG. 8: TGA profile of the amorphous 1 eq. hydrochloride salt (Sample No. Y11526-42-SU11-methanol-dichloromethane).

FIG. 9: ¹H-NMR spectrum of the amorphous 1 eq. hydrochloride salt (Sample No. Y11526-42-SU11-methanol-dichloromethane).

FIG. 10: PLM photograph of the amorphous 1 eq. hydrochloride salt (Sample No. Y11526-42-SU11-methanol-dichloromethane).

FIG. 11: XRPD pattern of the amorphous 2 eq. hydrochloride salt (Sample No. Y11526-28-SU5-methanol-dichloromethane).

FIG. 12: DSC profile of the amorphous 2 eq. hydrochloride salt (Sample No. Y11526-28-SU5-methanol-dichloromethane).

FIG. 13: TGA profile of the amorphous 2 eq. hydrochloride salt (Sample No. Y11526-28-SU5-methanol-dichloromethane).

FIG. 14: ¹H-NMR spectrum of the amorphous 2 eq. hydrochloride salt (Sample No. Y11526-28-SU5-methanol-dichloromethane).

FIG. 15: PLM photograph of the amorphous 2 eq. hydrochloride salt (Sample No. Y11526-28-SU5-methanol-dichloromethane).

FIG. 16: XRPD pattern of the amorphous 1 eq. mesylate salt (Sample No. Y11526-42-SU10-methanol-dichloromethane).

FIG. 17: DSC profile of the amorphous 1 eq. mesylate salt (Sample No. Y11526-42-SU10-methanol-dichloromethane).

FIG. 18: TGA profile of the amorphous 1 eq. mesylate salt (Sample No. Y11526-42-SU10-methanol-dichloromethane).

FIG. 19: ¹H-NMR spectrum of the amorphous 1 eq. mesylate salt (Sample No. Y11526-42-SU10-methanol-dichloromethane).

FIG. 20: PLM photograph of the amorphous 1 eq. mesylate salt (Sample No. Y11526-42-SU10-methanol-dichloromethane).

FIG. 21: XRPD pattern of the amorphous 2 eq. mesylate salt (Sample No. Y11526-28-SU4-methanol-dichloromethane).

FIG. 22: DSC profile of the amorphous 2 eq. mesylate salt (Sample No. Y11526-28-SU4-methanol-dichloromethane).

FIG. 23: TGA profile of the amorphous 2 eq. mesylate salt (Sample No. Y11526-28-SU4-methanol-dichloromethane).

FIG. 24: ¹H-NMR spectrum of the amorphous 2 eq. mesylate salt (Sample No. Y11526-28-SU4-methanol-dichloromethane).

FIG. 25: PLM photograph of the amorphous 2 eq. mesylate salt (Sample No. Y11526-28-SU4-methanol-dichloromethane).

FIG. 26: XRPD pattern of the amorphous 1 eq. besylate salt (Sample No. Y11526-42-SU9-methanol-dichloromethane).

FIG. 27: DSC profile of the amorphous 1 eq. besylate salt (Sample No. Y11526-42-SU9-methanol-dichloromethane).

FIG. 28: TGA profile of the amorphous 1 eq. besylate salt (Sample No. Y11526-42-SU9-methanol-dichloromethane).

FIG. 29: ¹H-NMR spectrum of the amorphous 1 eq. besylate salt (Sample No. Y11526-42-SU9-methanol-dichloromethane).

FIG. 30: PLM photograph of the amorphous 1 eq. besylate salt (Sample No. Y11526-42-SU9-methanol-dichloromethane).

FIG. 31: XRPD pattern of the amorphous 2 eq. besylate salt (Sample No. Y11526-30-SU1-water).

FIG. 32: DSC profile of the amorphous 2 eq. besylate salt (Sample No. Y11526-30-SU1-water).

FIG. 33: TGA profile of the amorphous 2 eq. besylate salt (Sample No. Y11526-30-SU1-water).

FIG. 34: ¹H-NMR spectrum of the amorphous 2 eq. besylate salt (Sample No. Y11526-30-SU1-water).

FIG. 35: PLM photograph of the amorphous 2 eq. besylate salt (Sample No. Y11526-30-SU1-water).

FIG. 36: XRPD pattern of the amorphous 1 eq. esylate salt (Sample No. Y11526-28-SU7-methanol-dichloromethane).

FIG. 37: DSC profile of the amorphous 1 eq. esylate salt (Sample No. Y11526-28-SU7-methanol-dichloromethane).

FIG. 38: TGA profile of the amorphous 1 eq. esylate salt (Sample No. Y11526-28-SU7-methanol-dichloromethane).

FIG. 39: ¹H-NMR spectrum of the amorphous 1 eq. esylate salt (Sample No. Y11526-28-SU7-methanol-dichloromethane).

FIG. 40: PLM photograph of the amorphous 1 eq. esylate salt (Sample No. Y11526-28-SU7-methanol-dichloromethane).

FIG. 41: XRPD pattern of the amorphous 1 eq. maleate salt (Sample No. Y11526-30-SU2-water).

FIG. 42: DSC profile of the amorphous 1 eq. maleate salt (Sample No. Y11526-30-SU2-water).

FIG. 43: TGA profile of the amorphous 1 eq. maleate salt (Sample No. Y11526-30-SU2-water).

FIG. 44: ¹H-NMR spectrum of the amorphous 1 eq. maleate salt (Sample No. Y11526-30-SU2-water).

FIG. 45: PLM photograph of the amorphous 1 eq. maleate salt (Sample No. Y11526-30-SU2-water).

FIG. 46: XRPD pattern of the crystalline 1 eq. hydrobromide salt (Sample No. Y11526-33-SU8-methanol-dichloromethane).

FIG. 47: DSC profile of the crystalline 1 eq. hydrobromide salt (Sample No. Y11526-33-SU8-methanol-dichloromethane).

FIG. 48: TGA profile of the crystalline 1 eq. hydrobromide salt (Sample No. Y11526-33-SU8-methanol-dichloromethane).

FIG. 49: ¹H-NMR spectrum of the crystalline 1 eq. hydrobromide salt (Sample No. Y11526-33-SU8-methanol-dichloromethane).

FIG. 50: PLM photograph of the crystalline 1 eq. hydrobromide salt (Sample No. Y11526-33-SU8-methanol-dichloromethane).

FIG. 51: XRPD pattern of the amorphous nitrate salt (Sample No. Y11526-18-RV7-methanol-dichloromethane).

FIG. 52: ¹H-NMR spectrum of the amorphous nitrate salt (Sample No. Y11526-18-RV7-methanol-dichloromethane).

FIG. 53: XRPD pattern of the amorphous sulfate salt (Sample No. Y11526-15-RV3-methanol-dichloromethane).

FIG. 54: DSC profile of the amorphous sulfate salt (Sample No. Y11526-15-RV3-methanol-acetonitrile).

FIG. 55: TGA profile of the amorphous sulfate salt (Sample No. Y11526-15-RV3-methanol-acetonitrile).

FIG. 56: ¹H-NMR spectrum of the amorphous sulfate salt (Sample No. Y11526-15-RV3-methanol-acetonitrile).

FIG. 57: PLM photograph of the amorphous sulfate salt (Sample No. Y11526-15-RV3-methanol-acetonitrile).

FIG. 58: XRPD pattern of the amorphous 2 eq. p-tosylate salt (Sample No. Y11526-23-FD11-water).

FIG. 59: DSC profile of the amorphous 2 eq. p-tosylate salt (Sample No. Y11526-23-FD11-water).

FIG. 60: TGA profile of the amorphous 2 eq. p-tosylate salt (Sample No. Y11526-23-FD11-water).

FIG. 61: ¹H-NMR spectrum of the amorphous 2 eq. p-tosylate salt (Sample No. Y11526-23-FD11-water).

FIG. 62: PLM photograph of the amorphous 2 eq. p-tosylate salt (Sample No. Y11526-23-FD11-water).

FIG. 63: photograph of the amorphous 2 eq. p-tosylate salt (Sample No. Y11526-23-FD11-water).

FIG. 64: XRPD pattern of the amorphous sulfosalicylate salt (Sample No. Y11526-25-FD13-1,4-dioxane).

FIG. 65: DSC profile of the amorphous sulfosalicylate salt (Sample No. Y11526-25-FD13-1,4-dioxane).

FIG. 66: TGA profile of the amorphous sulfosalicylate salt (Sample No. Y11526-25-FD13-1,4-dioxane).

FIG. 67: ¹H-NMR spectrum of the amorphous sulfosalicylate salt (Sample No. Y11526-25-FD13-1,4-dioxane).

FIG. 68: PLM photograph of the amorphous sulfosalicylate salt (Sample No. Y11526-25-FD13-1,4-dioxane).

FIG. 69: XRPD pattern of the amorphous sulfosalicylate salt (Sample No. Y11526-25-FD12-1,4-dioxane).

FIG. 70: DSC profile of the amorphous sulfosalicylate salt (Sample No. Y11526-25-FD12-1,4-dioxane).

FIG. 71: TGA profile of the amorphous sulfosalicylate salt (Sample No. Y11526-25-FD12-1,4-dioxane).

FIG. 72: ¹H-NMR spectrum of the amorphous sulfosalicylate salt (Sample No. Y11526-25-FD12-1,4-dioxane).

FIG. 73: PLM photograph of the amorphous sulfosalicylate salt (Sample No. Y11526-25-FD12-1,4-dioxane).

FIG. 74: XRPD pattern of the amorphous L-malate salt (Sample No. Y11526-17-RV6-methanol-dichloromethane).

FIG. 75: DSC profile of the amorphous L-malate salt (Sample No. Y11526-17-RV6-methanol-dichloromethane).

FIG. 76: TGA profile of the amorphous L-malate salt (Sample No. Y11526-17-RV6-methanol-dichloromethane).

FIG. 77: ¹H-NMR spectrum of the amorphous L-malate salt (Sample No. Y11526-17-RV6-methanol-dichloromethane).

FIG. 78: PLM photograph of the amorphous L-malate salt (Sample No. Y11526-17-RV6-methanol-dichloromethane).

FIG. 79: XRPD pattern of the amorphous 1 eq. citrate salt (Sample No. Y11526-10-FD6-1,4-dioxane).

FIG. 80: DSC profile of the amorphous 1 eq. citrate salt (Sample No. Y11526-10-FD6-1,4-dioxane).

FIG. 81: TGA profile of the amorphous 1 eq. citrate salt (Sample No. Y11526-10-FD6-1,4-dioxane).

FIG. 82: ¹H-NMR spectrum of the amorphous 1 eq. citrate salt (Sample No. Y11526-10-FD6-1,4-dioxane).

FIG. 83: PLM photograph of the amorphous 1 eq. citrate salt (Sample No. Y11526-10-FD6-1,4-dioxane).

FIG. 84: XRPD pattern of the amorphous 1 eq. L-tartrate salt (Sample No. Y11526-10-FD7-1,4-dioxane).

FIG. 85: DSC profile of the amorphous 1 eq. L-tartrate salt (Sample No. Y11526-10-FD7-1,4-dioxane).

FIG. 86: TGA profile of the amorphous 1 eq. L-tartrate salt (Sample No. Y11526-10-FD7-1,4-dioxane).

FIG. 87: ¹H-NMR spectrum of the amorphous 1 eq. L-tartrate salt (Sample No. Y11526-10-FD7-1,4-dioxane).

FIG. 88: HPLC chromatogram overlay of the amorphous 1 eq. L-tartrate salt (Sample No. Y11526-10-FD7-1,4-dioxane).

FIG. 89: XRPD overlay of the amorphous free base in a solid storage stability test, in which from top to bottom, the first trace is the XRPD of the crystalline free base as a control, the second trace is the solid obtained in Test BS2, the third trace is the XRPD of the solid obtained in Test BS1, and the fourth trace is the XRPD of the original solid.

FIG. 90: XRPD overlay of the amorphous 1 eq. hydrochloride salt in a solid storage stability test, in which the upper trace is the XRPD of the solid obtained in Test BS2, the middle trace is the XRPD of the solid obtained in Test BS1, and the lower trace is the XRPD of the original solid.

FIG. 91: XRPD overlay of the amorphous 2 eq. hydrochloride salt in a solid storage stability test, in which the upper trace is the XRPD of the solid obtained in Test BS2, the middle trace is the XRPD of the solid obtained in Test BS1, and the lower trace is the XRPD of the original solid.

FIG. 92: Photograph of the amorphous 2 eq. hydrochloride salt in a solid storage stability test, in which the left picture is the photograph of the original solid, and the right picture is the photograph of the solid obtained in Test BS1.

FIG. 93: XRPD overlay of the amorphous 1 eq. mesylate salt in a solid storage stability test, in which the upper trace is the XRPD of the solid obtained in Test BS2, the middle trace is the XRPD of the solid obtained in Test BS1, and the lower trace is the XRPD of the original solid.

FIG. 94: XRPD overlay of the amorphous 2 eq. mesylate salt in a solid storage stability test, in which the upper trace is the XRPD of the solid obtained in Test BS2, the middle trace is the XRPD of the solid obtained in Test BS1, and the lower trace is the XRPD of the original solid.

FIG. 95: Photograph of the amorphous 2 eq. mesylate salt in a solid storage stability test, in which the left picture is the photograph of the original solid, the middle picture is the photograph of the solid obtained in Test BS1, and the right picture is the photograph of the solid obtained in Test BS2.

FIG. 96: XRPD overlay of the amorphous 1 eq. besylate salt in a solid storage stability test, in which the upper trace is the XRPD of the solid obtained in Test BS2, the middle trace is the XRPD of the solid obtained in Test BS1, and the lower trace is the XRPD of the original solid.

FIG. 97: Photograph of the amorphous 2 eq. besylate salt in a solid storage stability test, in which the left picture is the photograph of the original solid, and the right picture is the photograph of the solid obtained in Test BS1.

FIG. 98: XRPD overlay of the amorphous 2 eq. besylate salt in a solid storage stability test, in which the upper trace is the XRPD of the solid obtained in Test BS2, and the lower trace is the XRPD of the original solid.

FIG. 99: Photograph of the amorphous 1 eq. esylate salt in a solid storage stability test, in which the left picture is the photograph of the original solid, and the right picture is the photograph of the solid obtained in Test BS1.

FIG. 100: XRPD overlay of the amorphous 1 eq. esylate salt in a solid storage stability test, in which the upper trace is the XRPD of the solid obtained in Test BS2, and the lower trace is the XRPD of the original solid.

FIG. 101: XRPD overlay of the amorphous 1 eq. maleate salt in a solid storage stability test, in which the upper trace is the XRPD of the solid obtained in Test BS2, the middle trace is the XRPD of the solid obtained in Test BS1, and the lower trace is the XRPD of the original solid.

FIG. 102: Photograph of the amorphous 1 eq. maleate salt in a solid storage stability test, in which the left picture is the photograph of the original solid, and the right picture is the photograph of the solid obtained in Test BS1.

FIG. 103: XRPD overlay of the crystalline 1 eq. hydrobromide salt in a solid storage stability test, in which the upper trace is the XRPD of the solid obtained in Test BS2, the middle trace is the XRPD of the solid obtained in Test BS1, and the lower trace is the XRPD of the original solid.

FIG. 104: XRPD pattern of the solid of the crystalline 1 eq. hydrobromide salt obtained in a 2 h solid solubility test, in which the upper trace is the XRPD of the solid obtained in the 2 h solid solubility test, and the lower trace is the XRPD of the original solid.

FIG. 105: X-ray powder diffraction pattern of crystalline form II-A of the mesylate salt.

FIG. 106: Thermogravimetric analysis (TGA)/differential scanning calorimetry (DSC) profile of crystalline form II-A of the mesylate salt.

FIG. 107: X-ray powder diffraction pattern of crystalline form III-A of the hydrochloride salt.

FIG. 108: Thermogravimetric analysis (TGA)/differential scanning calorimetry (DSC) profile of crystalline form III-A of the hydrochloride salt.

FIG. 109: X-ray powder diffraction pattern of crystalline form IV-C of the maleate salt.

FIG. 110: Thermogravimetric analysis (TGA)/differential scanning calorimetry (DSC) profile of crystalline form IV-C of the maleate salt.

### DETAILED DESCRIPTION OF THE INVENTION

In one aspect, the present application relates to a salt formed from a compound of formula (I) with an acid:

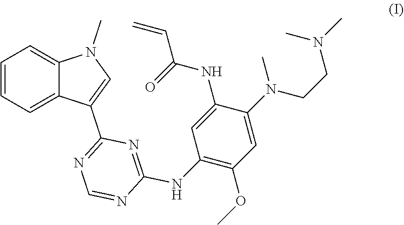 [SMILES: C=CC(=O)Nc1cc(Nc2ncnc(-c3cn(C)c4ccccc34)n2)c(OC)cc1N(C)CCN(C)C]

wherein the acid is selected from hydrochloric acid, methanesulfonic acid, benzenesulfonic acid, ethanesulfonic acid, maleic acid, hydrobromic acid, citric acid, L-tartaric acid, and p-toluenesulfonic acid. Preferably, the acid is selected from hydrochloric acid, methanesulfonic acid, and maleic acid. More preferably, the equivalent ratio of the compound of formula (I) to the acid is 1:1 or 1:2. Further preferably, the acid is methanesulfonic acid. More further preferably, the equivalent ratio of the compound of formula (I) to the acid is 1:1.

The inventors of the present application have found that when the compound of formula (I) is reacted with an organic or inorganic acid, it is unexpected that the compound of formula (I) can form salts with some acids, but cannot form salts with other acids at all.

More surprisingly, when the compound of formula (I) is reacted with an acid selected from hydrochloric acid, methanesulfonic acid, benzenesulfonic acid, ethanesulfonic acid, maleic acid, hydrobromic acid, sulfosalicylic acid, L-malic acid, citric acid, or L-tartaric acid in a molar charge ratio of compound of formula (I):acid of 1:1, some of the salts formed have a stoichiometric ratio of compound of formula (I):acid of 1:1, while the compound of formula (I) and some acids cannot form salts having a stoichiometric ratio of compound of formula (I):acid of 1:1. Specifically:
hydrochloric acid, methanesulfonic acid, benzenesulfonic acid, ethanesulfonic acid, maleic acid, hydrobromic acid, citric acid, and L-tartaric acid enable the resulting salts to have a stoichiometric ratio of compound of formula (I):acid of 1:1 (the corresponding salts are hereinafter referred to as 1 eq. hydrochloride salt, 1 eq. mesylate salt, 1 eq. besylate salt, 1 eq. esylate salt, 1 eq. maleate salt, 1 eq. hydrobromide salt, 1 eq. citrate salt and 1 eq. L-tartrate, respectively), while sulfosalicylic acid and L-malic acid cannot enable the resulting salts to have a stoichiometric ratio of compound of formula (I):acid of 1:1.

In addition, more surprisingly, when the compound of formula (I) is reacted with an acid selected from hydrochloric acid, methanesulfonic acid, benzenesulfonic acid, nitric acid, sulfuric acid, p-toluenesulfonic acid, or sulfosalicylic acid in a molar charge ratio of compound of formula (I):acid of 1:2, some of the salts formed have a stoichiometric ratio of compound of formula (I):acid of 1:2, while the compound of formula (I) and some acids cannot form salts having a stoichiometric ratio of compound of formula (I):acid of 1:2. Specifically:
hydrochloric acid, methanesulfonic acid, benzenesulfonic acid, and p-toluenesulfonic acid enable the resulting salts to have a stoichiometric ratio of compound of formula (I):acid of 1:2 (the corresponding salts are hereinafter referred to as 2 eq. hydrochloride salt, 2 eq. mesylate salt, 2 eq. besylate salt, and 2 eq. p-tosylate salt, respectively), while nitric acid, sulfuric acid, and sulfosalicylic acid cannot enable the resulting salts to have a stoichiometric ratio of compound of formula (I):acid of 1:2.

More specifically, the salts obtained as above include 1 eq. hydrochloride salt, 1 eq. mesylate salt, 1 eq. besylate salt, 1 eq. esylate salt, 1 eq. maleate salt, 1 eq. hydrobromide salt, 1 eq. citrate salt, 1 eq. L-tartrate, 2 eq. hydrochloride salt, 2 eq. mesylate salt, 2 eq. besylate salt and 2 eq. p-tosylate salt, and sulfosalicylate salt, L-malate salt, nitrate salt, and sulfate salt that do not have a reasonable salt-forming equivalent ratio. For example, the salts obtained as above include amorphous 1 eq. hydrochloride salt, amorphous 1 eq. mesylate salt, amorphous 1 eq. besylate salt, amorphous 1 eq. esylate salt, amorphous 1 eq. maleate salt, crystalline 1 eq. hydrobromide salt, amorphous 1 eq. citrate salt, amorphous 1 eq. L-tartrate, amorphous 2 eq. hydrochloride salt, amorphous 2 eq. mesylate salt, amorphous 2 eq. besylate salt and amorphous 2 eq. p-tosylate salt, and amorphous sulfosalicylate salt, amorphous L-malate salt, amorphous nitrate salt, and amorphous sulfate salt that do not have a reasonable salt-forming equivalent ratio.

It is crucial for drug development whether the salts obtained as above have chemical and physical stability at the end of the preparation. The chemical and physical stability of the salts at the end of the preparation includes:
the chemical stability of the salts at the end of the preparation, that is, the purity of the salts obtained at the end of the preparation is not significantly reduced compared with the purity of the free base used to prepare the salts; and
the physical stability of the salts at the end of the preparation, that is, the salts do not undergo crystal phase transformation, moisture absorption and/or color change, etc. immediately at the end of the preparation.

Different salts behave differently in terms of chemical and physical stability at the end of the preparation, indicating that it is unpredictable how a certain salt behaves. Specifically:
1 eq. citrate salt, 1 eq. L-tartrate, nitrate salt and L-malate salt are chemically unstable at the end of the preparation;
2 eq. p-tosylate salt and sulfate salt are physically unstable at the end of the preparation; and
1 eq. hydrochloride salt, 2 eq. hydrochloride salt, 1 eq. mesylate salt, 2 eq. mesylate salt, 1 eq. besylate salt, 2 eq. besylate salt, 1 eq. esylate salt, 1 eq. maleate salt, 1 eq. hydrobromide salt, and sulfosalicylate salt are chemically and physically stable at the end of the preparation.

The salts as above which have a reasonable salt-forming equivalent ratio and are chemically and physically stable at the end of the preparation include 1 eq. hydrochloride salt, 2 eq. hydrochloride salt, 1 eq. mesylate salt, 2 eq. mesylate salt, 1 eq. besylate salt, 2 eq. besylate salt, 1 eq. esylate salt, 1 eq. maleate salt, and 1 eq. hydrobromide salt. It is also crucial for drug development whether these salts remain chemically and physically stable after storage. The chemical and physical stability of the salts after storage includes:
the chemical stability of the salts after storage, that is, the purity of the salts after storage is not significantly reduced compared with the purity of the salts before storage; and
the physical stability of the salts after storage, that is, the salts do not undergo crystal phase transformation, moisture absorption and/or color change, etc. after storage.

Different salts behave differently in terms of chemical and physical stability after storage, indicating that it is unpredictable how a certain salt behaves. Specifically:
1 eq. hydrochloride salt, 1 eq. besylate salt, and 1 eq. maleate salt are chemically unstable under storage conditions of “solid/25° C./60% RH/open/1 week” and/or “solid/60° C./closed container/1 week”;
2 eq. hydrochloride salt, 2 eq. mesylate salt, 2 eq. besylate salt, 1 eq. esylate salt, and 1 eq. maleate salt are physically unstable under storage conditions of “solid/25° C./60% RH/open/1 week” and/or “solid/60° C./closed container/1 week”; and
1 eq. mesylate salt and 1 eq. hydrobromide salt are chemically and physically stable under storage conditions of “solid/25° C./60% RH/open/1 week” and “solid/60° C./closed container/1 week”.

The salts as above which have a reasonable salt-forming equivalent ratio and are chemically and physically stable at the end of the preparation include 1 eq. hydrochloride salt, 2 eq. hydrochloride salt, 1 eq. mesylate salt, 2 eq. mesylate salt, 1 eq. besylate salt, 2 eq. besylate salt, 1 eq. esylate salt, 1 eq. maleate salt, and 1 eq. hydrobromide salt. It is also crucial for drug development whether these salts have better solubility. Different salts have different solubility, indicating that it is unpredictable what solubility a certain salt may have. Specifically:
2 eq. besylate salt and 1 eq. hydrobromide salt do not achieve a solubility of 2 mg/mL in some solvents that mimic physiological conditions; and
1 eq. hydrochloride salt, 2 eq. hydrochloride salt, 1 eq. mesylate salt, 2 eq. mesylate salt, 1 eq. besylate salt, 1 eq. esylate salt, and 1 eq. maleate salt have a solubility greater than 2 mg/mL in various solvents that mimic physiological conditions.

As can be seen from the above contents, 1 eq. mesylate salt surprisingly and unexpectedly has a reasonable salt-forming equivalent ratio, better chemical stability, better physical stability and better solubility simultaneously, which makes it as a salt of the compound of formula (I) suitable for further drug development.

In another aspect according to the invention, the present application relates to a pharmaceutical composition comprising the salt according to the invention, which comprises the salt according to the invention and a pharmaceutically acceptable carrier.

In still another aspect according to the invention, the present application relates to a method of treating non-small cell lung cancer (NSCLC) with EGFR exon 20 insertion mutation, comprising administering the salt according to the invention to a patient.

In a further aspect according to the invention, the present application relates to the use of the salt according to the invention in the preparation of a medicament for the treatment of non-small cell lung cancer (NSCLC) with EGFR exon 20 insertion mutation.

In addition to the properties brought about by the salt formation, since crystallization may provide the salt with additional properties that are more suitable for further drug development, such as better stability, processability, and druggability, crystallization studies were carried out on some of the salts prepared after the above salt formation study and screening of the compound of formula (I). After conducting crystallization studies on the prepared salts, the inventors of the present application have found that the mesylate, hydrochloride salt and maleate salts of the compound of formula (I) can form crystals and have obtained the crystalline forms of the salts, and have found that the crystalline forms of the mesylate, hydrochloride salt and maleate salts of the compound of formula (I) have improved bioavailability and increased dynamic solubility.

The present invention therefore provides the following technical solutions relating to the crystalline forms.

In one aspect according to the invention, the present application relates to a crystalline form II-A of the mesylate salt of the compound of formula (I), characterized in that the crystalline form II-A at least has an X-ray powder diffraction pattern having characteristic peaks expressed in 2θ° of 15.12°±0.2°, 22.28°±0.2° and 25.82°±0.2°.

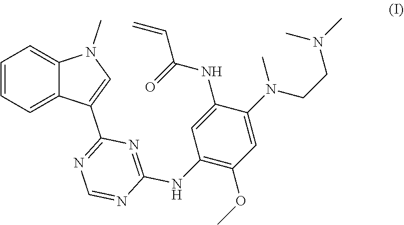 [SMILES: C=CC(=O)Nc1cc(Nc2ncnc(-c3cn(C)c4ccccc34)n2)c(OC)cc1N(C)CCN(C)C]

Further, the above crystalline form II-A is characterized in that the crystalline form II-A at least has an X-ray powder diffraction pattern having characteristic peaks expressed in 2θ° of one or all of 19.62°±0.2° and 26.24°±0.2°.

Further, the above crystalline form II-A is characterized in that the crystalline form II-A at least has an X-ray powder diffraction pattern having characteristic peaks expressed in 2θ° of one or all of 10.36°±0.2° and 18.94°±0.2°.

Further, the above crystalline form II-A is characterized in that the crystalline form II-A at least has an X-ray powder diffraction pattern having characteristic peaks expressed in 2θ° of one or all of 7.22°±0.2°, 8.62°±0.2°, 9.48°±0.2°, 15.74°±0.2°, 16.46°±0.2°, 16.92°±0.2°, 17.70°±0.2°, 19.30°±0.2°, 20.36°±0.2°, 20.81°±0.2°, 24.06°±0.2°, 24.78°±0.2°, and 25.54°±0.2°.

Further, the above crystalline form II-A is characterized in that the X-ray powder diffraction pattern thereof has characteristic peaks at diffraction angle 2θ° of 7.22°±0.2°, 8.62°±0.2°, 9.48°±0.2°, 10.36°±0.2°, 15.12°±0.2°, 15.74°±0.2°, 16.46°±0.2°, 16.92°±0.2°, 17.70°±0.2°, 18.94°±0.2°, 19.30°±0.2°, 19.62°±0.2°, 20.36°±0.2°, 20.81°±0.2°, 22.28°±0.2°, 24.06°±0.2°, 24.78°±0.2°, 25.54°±0.2°, 25.82°±0.2°, and 26.24°±0.2°.

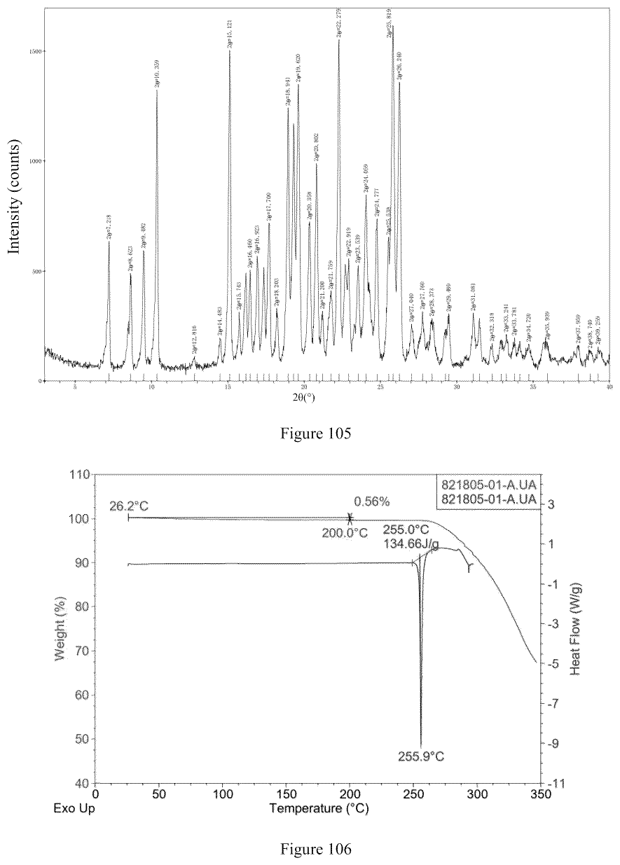

Further, the above crystalline form II-A is characterized in that the X-ray powder diffraction pattern of the crystalline form II-A is substantially as shown in FIG. 105.

Further, the above crystalline form II-A is characterized in that the crystalline form II-A has a weight loss of 0.56% at 200.0° C.

Further, the above crystalline form II-A is characterized in that the crystalline form II-A begins to show an endothermic peak when heated to 255.9° C.

Further, the above crystalline form II-A is characterized in that its thermogravimetric analysis (TGA)/differential scanning calorimetry (DSC) profile is substantially as shown in FIG. 106.

Further, the above crystalline form II-A is characterized in that the molar ratio of the compound of formula (I) to methanesulfonic acid is 1:1.

In addition, the present invention provides a method for preparing the crystalline form II-A of the mesylate salt of the compound of formula (I), comprising:
a) suspending the compound of formula (I) in a first solvent;
b) increasing the temperature to 20-80° C., and adding dropwise a methanesulfonic acid solution dissolved in a second solvent beforehand; and
c) crystallizing the mixture and filtrating to obtain the crystalline form II-A.

Further, the first solvent is water, ketone, cyclic ether or nitrile solvent or a mixed solvent thereof; and the second solvent is water, ketone, cyclic ether or nitrile solvent or a mixed solvent thereof. Yet further, the first solvent is water, ketone, cyclic ether or nitrile solvent or a mixed solvent thereof; and the second solvent is a ketone, cyclic ether or nitrile solvent, or a mixed solvent of water and a ketone, cyclic ether or nitrile solvent. Yet further, the ketone solvent includes, but is not limited to, acetone, the cyclic ether solvent includes, but is not limited to, tetrahydrofuran or 1,4-dioxane, and the nitrile solvent includes, but is not limited to, acetonitrile.

Further, in the mixed solvent of the ketone, cyclic ether or nitrile solvent and water, the volume ratio of the ketone, cyclic ether or nitrile solvent to water is 5:1-25:1, and still further, the volume ratio of the ketone, cyclic ether or nitrile solvent to water is 9:1-15:1.

Further, the temperature is increased in step b) to 20-50° C.

The present invention provides a method for preparing the crystalline form II-A of the mesylate salt of the compound of formula (I), comprising:
a) suspending the compound of formula (I) in a first solvent;
b) increasing the temperature to 20-80° C., and adding dropwise a methanesulfonic acid solution dissolved in a second solvent beforehand; and
c) adding dropwise a third solvent;
d) crystallizing the mixture and filtrating to obtain the crystalline form II-A.

Further, the first solvent is water, ketone, cyclic ether or nitrile solvent or a mixed solvent thereof; and the second solvent is water, ketone, cyclic ether or nitrile solvent or a mixed solvent thereof. Yet further, the first solvent is water, ketone, cyclic ether or nitrile solvent or a mixed solvent thereof; and the second solvent is a ketone, cyclic ether or nitrile solvent, or a mixed solvent of water and a ketone, cyclic ether or nitrile solvent. Yet further, the ketone solvent includes, but is not limited to, acetone, the cyclic ether solvent includes, but is not limited to, tetrahydrofuran or 1,4-dioxane, and the nitrile solvent includes, but is not limited to, acetonitrile.

Further, in the mixed solvent of the ketone, cyclic ether or nitrile solvent and water, the volume ratio of the ketone, cyclic ether or nitrile solvent to water is 5:1-25:1, and still further, the volume ratio of the ketone, cyclic ether or nitrile solvent to water is 9:1-16:1.

Further, the temperature is increased in step b) to 20-50° C.

Further, the third solvent is a C₆₋₇ alkane, ether or ester solvent. Yet further, the C₆₋₇ alkane solvent includes, but is not limited to, n-heptane; the ether solvent includes, but is not limited to, methyl tert-butyl ether; and the ester solvent includes, but is not limited to, methyl formate, ethyl acetate, isopropyl acetate, propyl acetate, or butyl acetate.

In one aspect, the present application relates to a crystalline form III-A of the hydrochloride salt of the compound of formula (I), characterized in that the crystalline form III-A at least has an X-ray powder diffraction pattern having characteristic peaks expressed in 2θ° of 24.85°±0.2°, 14.08°±0.2° and 14.45°±0.2°.

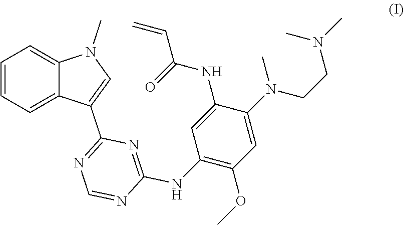 [SMILES: C=CC(=O)Nc1cc(Nc2ncnc(-c3cn(C)c4ccccc34)n2)c(OC)cc1N(C)CCN(C)C]

Further, the above crystalline form III-A is characterized in that the crystalline form III-A at least has an X-ray powder diffraction pattern having characteristic peaks expressed in 2θ° of one or all of 25.87°±0.2 and 6.55°±0.2°.

Further, the above crystalline form III-A is characterized in that the crystalline form III-A at least has an X-ray powder diffraction pattern having characteristic peaks expressed in 2θ° of one or all of 13.10°±0.2° and 19.07°±0.2°.

Further, the above crystalline form III-A is characterized in that the crystalline form III-A at least has an X-ray powder diffraction pattern having characteristic peaks expressed in 2θ° of one or all of 10.36°±0.2°, 12.28°±0.2°, 15.98°±0.2°, 17.33°±0.2°, 19.57°±0.2°, 21.15°±0.2°, 21.72°±0.2°, 24.30°±0.2°, and 33.29°±0.2°.

Further, the above crystalline form III-A is characterized in that the X-ray powder diffraction pattern thereof has characteristic peaks at diffraction angle 2θ° of 6.55°±0.2°, 10.36°±0.2°, 12.28°±0.2°, 13.10°±0.2°, 14.08°±0.2°, 14.45°±0.2°, 15.98°±0.2°, 17.33°±0.2°, 19.07°±0.2°, 19.57°±0.2°, 21.15°±0.2°, 21.72°±0.2°, 24.30°±0.2°, 24.85°±0.2°, 25.87°±0.2°, and 33.29°±0.2°.

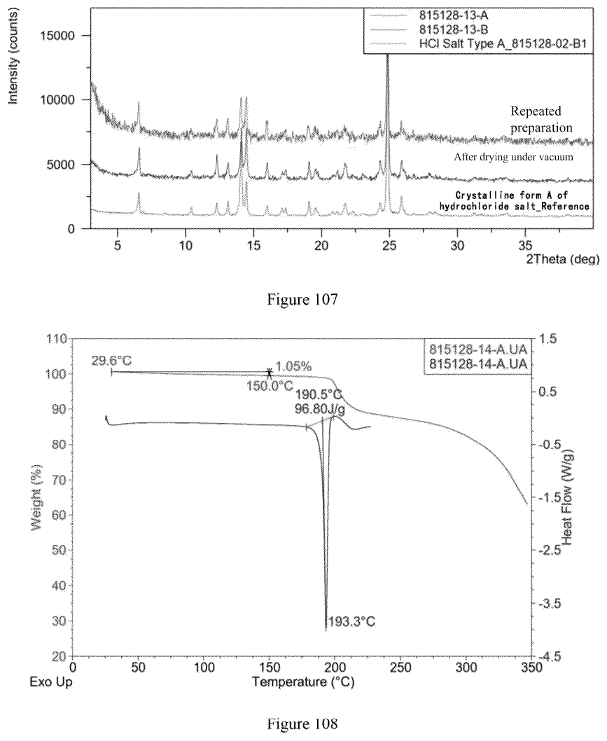

Further, the above crystalline form III-A is characterized in that the X-ray powder diffraction pattern of the crystalline form III-A is substantially as shown in FIG. 107.

Further, the above crystalline form III-A is characterized in that the crystalline form III-A has a weight loss of 1.05% at 150.0° C.

Further, the above crystalline form III-A is characterized in that the crystalline form III-A begins to show an endothermic peak when heated to 193.3° C.

Further, the above crystalline form III-A is characterized in that its thermogravimetric analysis (TGA)/differential scanning calorimetry (DSC) profile is substantially as shown in FIG. 108.

Further, the above crystalline form III-A is characterized in that the molar ratio of the compound of formula (I) to hydrochloric acid is 1:1.

In addition, the present invention provides a method for preparing the crystalline form III-A of the hydrochloride salt of the compound of formula (I), comprising:
a) suspending the compound of formula (I) in a first solvent;
b) increasing the temperature to 20-80° C., and adding dropwise a concentrated hydrochloric acid (about 37%) solution dissolved in a second solvent beforehand; and
c) crystallizing the mixture and filtrating to obtain the crystalline form III-A. Further, the first solvent is water, ketone, cyclic ether or nitrile solvent or a mixed solvent thereof; and the second solvent is water, ketone, cyclic ether or nitrile solvent or a mixed solvent thereof. Yet further, the first solvent is water, ketone, cyclic ether or nitrile solvent or a mixed solvent thereof; and the second solvent is an alcohol or nitrile solvent, or a mixed solvent of water and an alcohol or nitrile solvent. Yet further, the ketone solvent includes, but is not limited to, acetone, the cyclic ether solvent includes, but is not limited to, tetrahydrofuran or 1,4-dioxane, the alcohol solvent includes, but is not limited to, ethanol, and the nitrile solvent includes, but is not limited to, acetonitrile.

Further, in the mixed solvent of the alcohol or nitrile solvent and water, the volume ratio of the alcohol or nitrile solvent to water is 5:1-25:1, and still further, the volume ratio of the alcohol or nitrile solvent to water is 9:1-15:1.

Further, the temperature is increased in step b) to 20-50° C.

The present invention provides a method for preparing the crystalline form III-A of the hydrochloride salt of the compound of formula (I), comprising:
a) suspending the compound of formula (I) in a first solvent;
b) increasing the temperature to 20-80° C., and adding dropwise a concentrated hydrochloric acid (about 37%) solution dissolved in a second solvent beforehand; and
c) adding dropwise a third solvent;
d) crystallizing the mixture and filtrating to obtain the crystalline form III-A.

Further, the first solvent is water, ketone, cyclic ether or nitrile solvent or a mixed solvent thereof; and the second solvent is water, ketone, cyclic ether or nitrile solvent or a mixed solvent thereof. Yet further, the first solvent is water, ketone, cyclic ether or nitrile solvent or a mixed solvent thereof; and the second solvent is an alcohol or nitrile solvent, or a mixed solvent of water and an alcohol or nitrile solvent. Yet further, the ketone solvent includes, but is not limited to, acetone, the cyclic ether solvent includes, but is not limited to, tetrahydrofuran or 1,4-dioxane, the alcohol solvent includes, but is not limited to, ethanol, and the nitrile solvent includes, but is not limited to, acetonitrile.

Further, in the mixed solvent of the alcohol or nitrile solvent and water, the volume ratio of the alcohol or nitrile solvent to water is 5:1-25:1, and still further, the volume ratio of the alcohol or nitrile solvent to water is 9:1-15:1.

Further, the temperature is increased in step b) to 20-50° C.

Further, the third solvent is a C₆₋₇ alkane, ether or ester solvent. Yet further, the C₆₋₇ alkane solvent includes, but is not limited to, n-heptane; the ether solvent includes, but is not limited to, methyl tert-butyl ether; and the ester solvent includes, but is not limited to, methyl formate, ethyl acetate, isopropyl acetate, propyl acetate, or butyl acetate.

In one aspect, the present application relates to a crystalline form IV-C of the maleate salt of the compound of formula (I), characterized in that the crystalline form IV-C at least has an X-ray powder diffraction pattern having characteristic peaks expressed in 2θ° of 20.71°±0.2°, 25.76°±0.2° and 17.00°±0.2°.

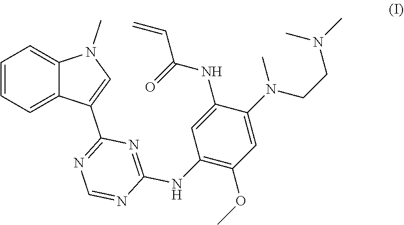 [SMILES: C=CC(=O)Nc1cc(Nc2ncnc(-c3cn(C)c4ccccc34)n2)c(OC)cc1N(C)CCN(C)C]

Further, the above crystalline form IV-C is characterized in that the crystalline form IV-C at least has an X-ray powder diffraction pattern having characteristic peaks expressed in 2θ° of one or all of 6.47°±0.2° and 10.63°±0.2°.

Further, the above crystalline form IV-C is characterized in that the crystalline form IV-C at least has an X-ray powder diffraction pattern having characteristic peaks expressed in 2θ° of one or all of 28.70°±0.2° and 16.42°±0.2°.

Further, the above crystalline form IV-C is characterized in that the crystalline form IV-C at least has an X-ray powder diffraction pattern having characteristic peaks expressed in 2θ° of one or all of 8.44°±0.2°, 9.15°±0.2°, 12.70°±0.2°, 15.48°±0.2°, 18.19°±0.2°, 22.43°±0.2°, and 31.39°±0.2°.

Further, the above crystalline form IV-C is characterized in that the X-ray powder diffraction pattern thereof has characteristic peaks at diffraction angle 2θ° of 6.47°±0.2°, 8.44°±0.2°, 9.15°±0.2°, 10.63°±0.2°, 12.70°±0.2°, 15.48°±0.2°, 16.42°±0.2°, 17.00°±0.2°, 18.19°±0.2°, 20.71°±0.2°, 22.43°±0.2°, 25.76°±0.2°, 28.70°±0.2°, and 31.39°±0.2°.

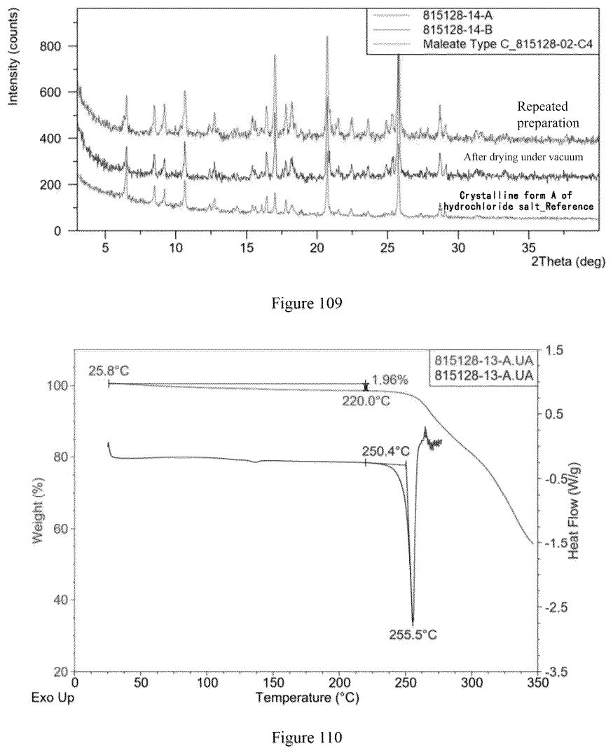

Further, the above crystalline form IV-C is characterized in that the X-ray powder diffraction pattern of the crystalline form IV-C is substantially as shown in FIG. 109.

Further, the above crystalline form IV-C is characterized in that the crystalline form IV-C has a weight loss of 1.96% at 220.0° C.

Further, the above crystalline form IV-C is characterized in that the crystalline form IV-C begins to show an endothermic peak when heated to 255.5° C.

Further, the above crystalline form IV-C is characterized in that its thermogravimetric analysis (TGA)/differential scanning calorimetry (DSC) profile is substantially as shown in FIG. 110.

Further, the above crystalline form IV-C is characterized in that the molar ratio of the compound of formula (I) to maleic acid is 1:1.

In addition, the present invention provides a method for preparing the crystalline form IV-C of the maleate of the compound of formula (I), comprising:
a) suspending the compound of formula (I) in a first solvent;
b) increasing the temperature to 20-80° C., and adding dropwise a maleic acid solution dissolved in a second solvent beforehand; and
c) crystallizing the mixture and filtrating to obtain the crystalline form IV-C.

Further, the first solvent is water, ketone, cyclic ether or nitrile solvent or a mixed solvent thereof; and the second solvent is water, ketone, cyclic ether or nitrile solvent or a mixed solvent thereof. Yet further, the first solvent is water, ketone, cyclic ether or nitrile solvent or a mixed solvent thereof; and the second solvent is a ketone, cyclic ether or nitrile solvent, or a mixed solvent of water and a ketone, cyclic ether or nitrile solvent. Yet further, the ketone solvent includes, but is not limited to, acetone, the cyclic ether solvent includes, but is not limited to, tetrahydrofuran or 1,4-dioxane, and the nitrile solvent includes, but is not limited to, acetonitrile.

Further, in the mixed solvent of the ketone, cyclic ether or nitrile solvent and water, the volume ratio of the ketone, cyclic ether or nitrile solvent to water is 5:1-25:1, and still further, the volume ratio of the ketone, cyclic ether or nitrile solvent to water is 9:1-15:1.

Further, the temperature is increased in step b) to 20-50° C.

The present invention provides a method for preparing the crystalline form IV-C of the maleate of the compound of formula (I), comprising:
a) suspending the compound of formula (I) in a first solvent;
b) increasing the temperature to 20-80° C., and adding dropwise a maleic acid solution dissolved in a second solvent beforehand; and
c) adding dropwise a third solvent;
d) crystallizing the mixture and filtrating to obtain the crystalline form IV-C.

Further, the first solvent is water, ketone, cyclic ether or nitrile solvent or a mixed solvent thereof; and the second solvent is water, ketone, cyclic ether or nitrile solvent or a mixed solvent thereof. Yet further, the first solvent is water, ketone, cyclic ether or nitrile solvent or a mixed solvent thereof; and the second solvent is a ketone, cyclic ether or nitrile solvent, or a mixed solvent of water and a ketone, cyclic ether or nitrile solvent. Yet further, the ketone solvent includes, but is not limited to, acetone, the cyclic ether solvent includes, but is not limited to, tetrahydrofuran or 1,4-dioxane, and the nitrile solvent includes, but is not limited to, acetonitrile.

Further, in the mixed solvent of the ketone, cyclic ether or nitrile solvent and water, the volume ratio of the ketone, cyclic ether or nitrile solvent to water is 5:1-25:1, and still further, the volume ratio of the ketone, cyclic ether or nitrile solvent to water is 9:1-16:1.

Further, the temperature is increased in step b) to 20-50° C.

Further, the third solvent is a C₆₋₇ alkane, ether or ester solvent. Yet further, the C₆₋₇ alkane solvent includes, but is not limited to, n-heptane; the ether solvent includes, but is not limited to, methyl tert-butyl ether; and the ester solvent includes, but is not limited to, methyl formate, ethyl acetate, isopropyl acetate, propyl acetate, or butyl acetate.

Since the formation of the crystalline form is accidental and unpredictable, the obtaining of the crystalline form II-A of the mesylate salt of the compound of formula (I), the crystalline form III-A of the hydrochloride salt of the compound of formula (I) and the crystalline form IV-C of the maleate salt of the compound of formula (I) is in itself surprising and unexpected. The inventors of the present application have also surprisingly and unexpectedly discovered that these crystalline forms have improved bioavailability and increased dynamic solubility.

In one aspect, the present application relates to a method for preparing the compound of formula (I), comprising the following steps:

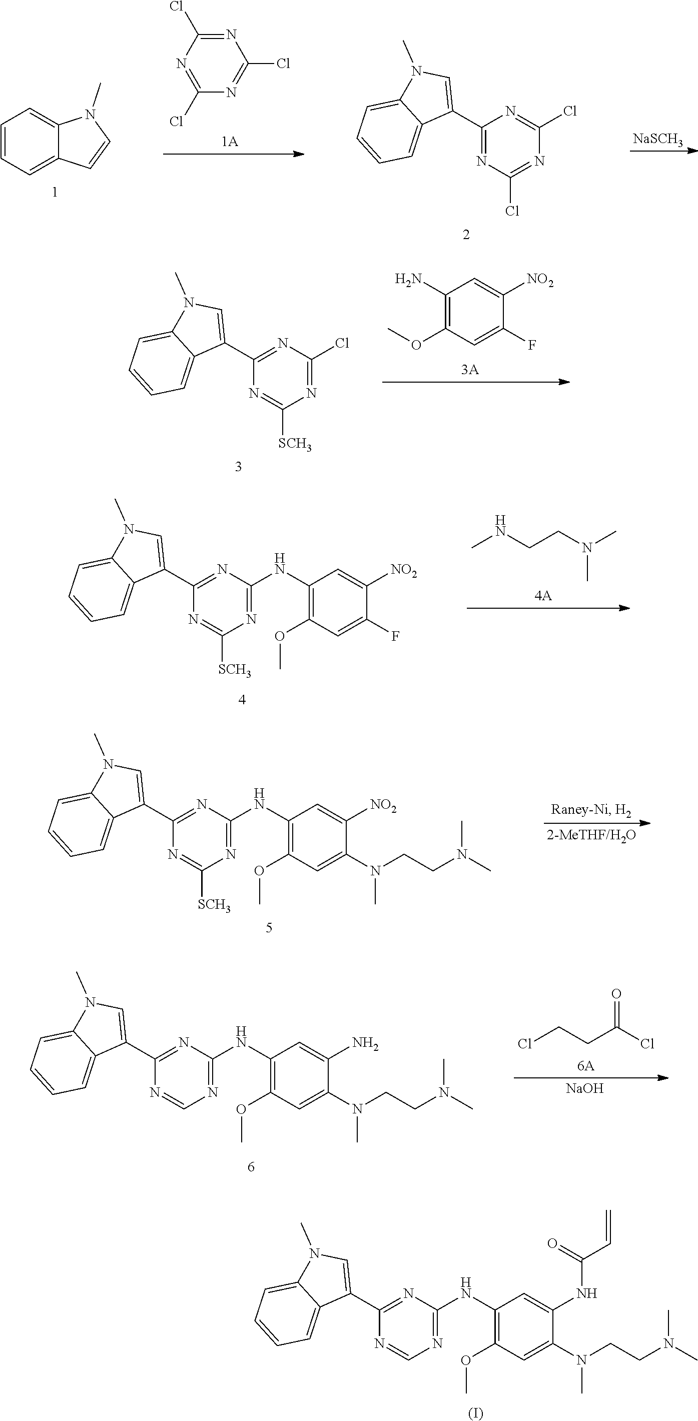

Further, the method for preparing the compound of formula (I) is as follows:

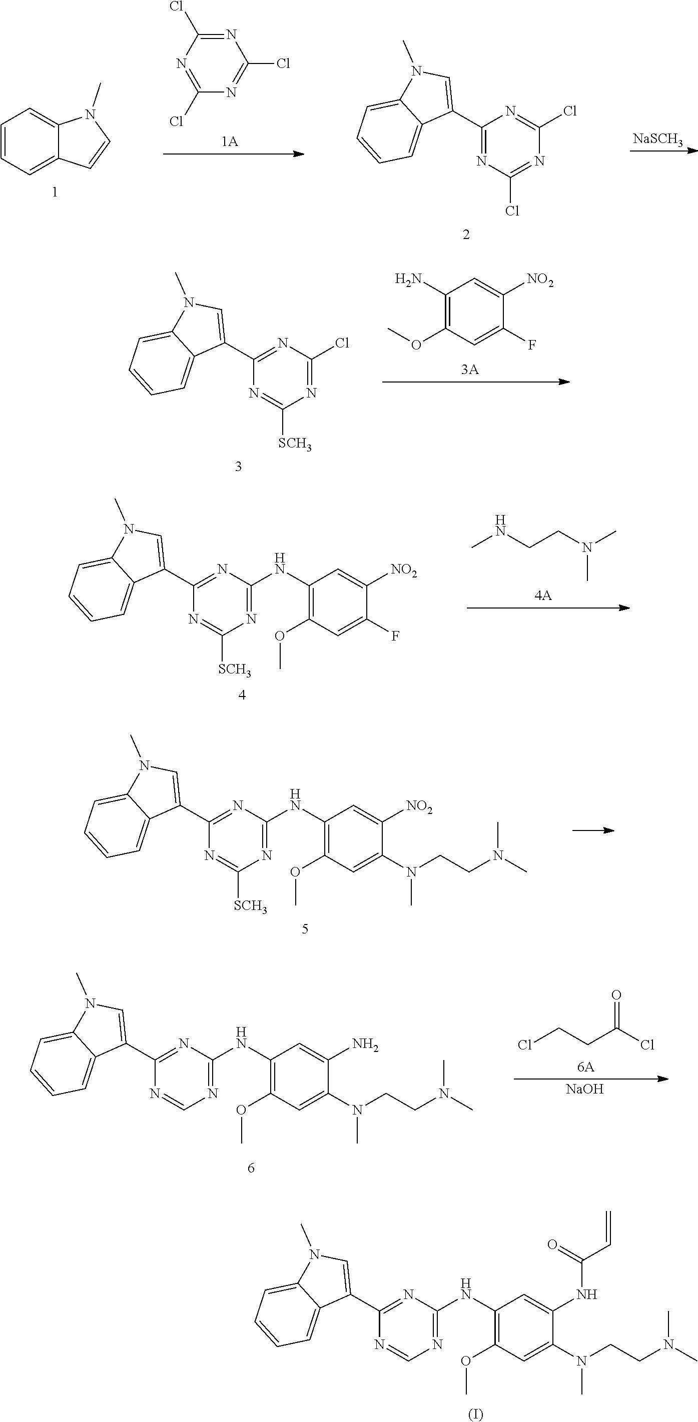

Compound 1A and Compound 1 are subjected to a substitution or coupling reaction to give Compound 2. Compound 2 is reacted with sodium thiomethoxide to give Compound 3. Compound 3 and Compound 3A are subjected to a substitution or coupling reaction to give Compound 4, which is then subjected to a nucleophilic substitution reaction with Compound 4A to give Compound 5. The nitro group and methylthio group of Compound 5 are sequentially reduced and removed to obtain Compound 6. Compound 6 and 3-chloropropionyl chloride 6A are subjected to acylation and elimination under basic conditions to give the compound of formula (I). The reducing agents employed in the method for reducing the nitro group are those well known in the art, including, but not limited to, iron powder, zinc powder, sodium sulfide, palladium carbon (Pd/C)/hydrogen, Raney-Ni/hydrogen, platinum dioxide/hydrogen, etc.

In one aspect according to the invention, the present application relates to a method for preparing the mesylate salt of the compound of formula (I), comprising the following steps:

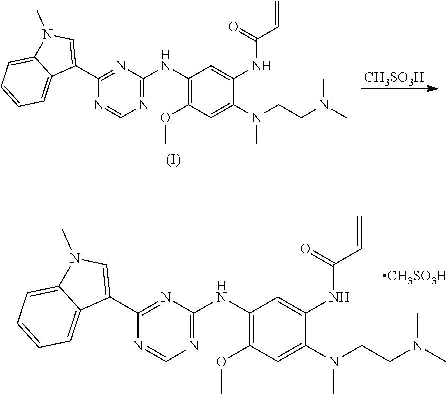

Further, the compound of formula (I) is directly reacted with methanesulfonic acid in a solvent to form a salt to obtain the mesylate salt of the compound of formula (I), and the solvent includes, but is not limited to, a mixed solvent of acetone and water.

In one aspect according to the invention, the present application relates to a method for preparing the hydrochloride salt of the compound of formula (I), comprising the following steps:

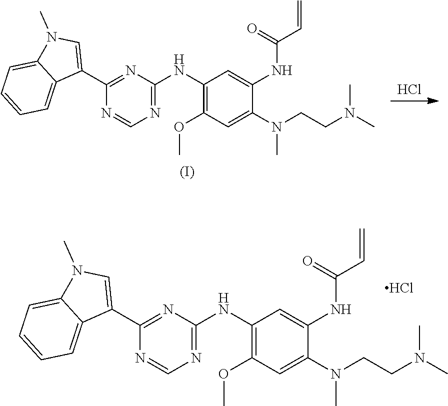

Further, the compound of formula (I) is directly reacted with hydrochloric acid in a solvent to form a salt to obtain the hydrochloride salt of the compound of formula (I), and the solvent includes, but is not limited to, a mixed solvent of ethanol and water.

In one aspect according to the invention, the present application relates to a method for preparing the maleate salt of the compound of formula (I), comprising the following steps:

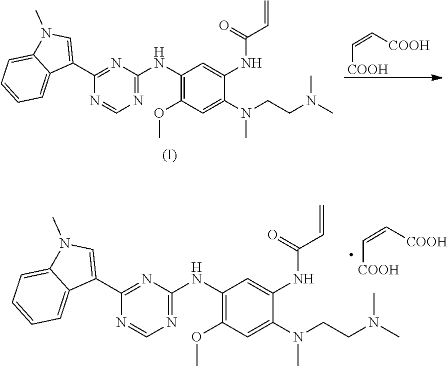

Further, the compound of formula (I) is directly reacted with maleic acid in a solvent to form a salt to obtain the maleate salt of the compound of formula (I), and the solvent includes, but is not limited to, a mixed solvent of ethanol and water.

The present invention provides a pharmaceutical composition comprising any one of the above salts or crystalline forms of the compound of formula (I) and a pharmaceutically acceptable carrier.

The present invention also provides any one of the above salts or crystalline forms of the compound of formula (I) for use as an antitumor agent.

The present invention also provides the use of any one of the above salts or crystalline forms of the compound of formula (I) in the preparation of a medicament for the treatment of diseases, especially cancer, mediated by EGFR activating, drug resistance or exon 20 insertion mutation in mammals, especially humans.

The present invention also provides the use of any one of the above salts or crystalline forms of the compound of formula (I) in the preparation of a medicament for the treatment of cancer.

The present invention also provides the use of any one of the above salts or crystalline forms of the compound of formula (I) for the treatment of diseases, especially cancer, mediated by EGFR activating, drug resistance or exon 20 insertion mutation in mammals, especially humans.

The present invention also provides a method for the treatment of diseases, especially cancer, mediated by EGFR activating, drug resistance or exon 20 insertion mutation in mammals, especially humans, comprising administering to a patient any one of the above salts or crystalline forms of the compound of formula (I) or a pharmaceutical composition comprising a therapeutically effective amount of any one of the above salts or crystalline forms of the compound of formula (I) and a pharmaceutically acceptable carrier, excipient or diluent.

The present invention also provides a method for the treatment of cancer, comprising administering to a patient any one of the above salts or crystalline forms of the compound of formula (I) or a pharmaceutical composition comprising a therapeutically effective amount of any one of the above salts or crystalline forms of the compound of formula (I) and a pharmaceutically acceptable carrier, excipient or diluent.

The cancer mentioned in the present invention includes, but is not limited to, for example ovarian cancer, non-small cell lung cancer, small cell lung cancer, cervical cancer, colorectal cancer, breast cancer, pancreatic cancer, glioma, glioblastoma, melanoma, prostatic cancer, leukemia, lymphoma, non-Hodgkin lymphoma, gastric cancer, lung cancer, hepatocellular carcinoma, gastrointestinal stromal tumor, thyroid cancer, bile duct cancer, endometrial cancer, kidney cancer, anaplastic large cell lymphoma, acute myeloid leukemia, multiple myeloma, or mesothelioma. The above salts or crystalline forms of the compound of formula (I) has better application especially for tumor types with epidermal growth factor receptor exon 20 insertion mutation. Further, the cancer is non-small cell lung cancer.

The epidermal growth factor receptor EGFR exon 20 insertion mutation mentioned in the present invention include, but are not limited to, V774insHV insertion mutation, D770insNPG insertion mutation, N771insH insertion mutation, V769insASV insertion mutation, A763insFQEA insertion mutation, D770insSVD insertion mutation and the like. For example, any one of the above salts or crystalline forms of the compound of formula (I) of the present invention can be used as an agent for the treatment of non-small cell lung cancer with epidermal growth factor receptor exon 20 insertion mutation.

Any one of the above salts or crystalline forms of the compound of formula (I) of the present invention can be administered to mammals, including humans, by oral, rectal, parenteral (intravenous, intramuscular, or subcutaneous), topical (dust, ointment, or drops), or intratumoral administration.

The dosage of any one of the above salts or crystalline forms of the compound of formula (I) of the present invention may be about 0.05-50 mg/kg body weight/day, for example 0.1-45 mg/kg body weight/day, more for example 0.5-35 mg/kg body weight/day.

Any one of the above salts or crystalline forms of the compound of formula (I) of the present invention can be formulated into solid preparations for oral administration, including, but not limited to, capsules, tablets, pills, powders and granules. In these solid dosage forms, any one of the above salts or crystalline forms of the compound of formula (I) of the present invention are mixed as an active ingredient with at least one conventional inert excipient (or carrier), such as sodium citrate or dicalcium phosphate, or mixed with the following ingredients: (1) fillers or compatibilizers, such as starch, lactose, sucrose, glucose, and mannitol; (2) binders, such as hydroxymethyl cellulose, alginate, gelatin, polyvinylpyrrolidone, sucrose, and gum arabic; (3) humectants, such as glycerin; (4) disintegrating agents, such as agar, calcium carbonate, potato starch or cassava starch, and alginic acid; (5) retarding solvents, such as paraffin; (6) absorption accelerators, such as quaternary ammonium compounds; (7) wetting agents, such as cetyl alcohol and glyceryl monostearate; (8) adsorbents, such as kaolin; and (9) lubricating agents, such as talc, calcium stearate, magnesium stearate, solid polyethylene glycol, and sodium lauryl sulfate, or mixtures thereof. Buffers may also be included in capsules, tablets and pills.

The solid dosage forms such as tablets, dragees, capsules, pills and granules can be coated or microencapsulated with coatings and shell materials such as enteric coatings and other materials well known in the art. They may contain opacifying agents, and the release of the active ingredient in such compositions may be in a certain part of the digestive tract in a delayed manner. Examples of embedding components that can be employed are polymeric substances and waxes. If desired, the active ingredient can also be in microencapsulated form with one or more of the above excipients.

Any one of the above salts or crystalline forms of the compound of formula (I) of the present invention can be formulated into liquid dosage forms for oral administration, including, but not limited to, pharmaceutically acceptable emulsions, solutions, suspensions, syrups, and tinctures. In addition to any one of the above salts or crystalline forms of the compound of formula (I) as the active ingredient, the liquid dosage forms may contain inert diluents conventionally employed in the art, such as water and other solvents, solubilizers and emulsifiers, such as ethanol, isopropanol, ethyl carbonate, ethyl acetate, propylene glycol, 1,3-butanediol, dimethylformamide, and oils, especially cottonseed oil, peanut oil, corn germ oil, olive oil, castor oil and sesame oil, or mixtures of these substances. In addition to these inert diluents, the liquid preparations of the present invention may also contain conventional adjuvants such as wetting agents, emulsifying agents, suspending agents, sweetening agents, flavoring agents and perfuming agents.

Such suspending agents include, for example, ethoxylated isostearyl alcohols, polyoxyethylene sorbitol and sorbitan, microcrystalline cellulose, aluminum methoxide, agar, or mixtures thereof.

Any one of the above salts or crystalline forms of the compound of formula (I) of the present invention may be formulated into dosage forms for parenteral injection, including, but not limited to, physiologically acceptable sterile aqueous or anhydrous solutions, dispersions, suspensions or emulsions, and sterile powders for reconstitution into sterile injectable solutions or dispersions. Suitable carriers, diluents, solvents or excipients include water, ethanol, polyols and suitable mixtures thereof.

Any one of the above salts or crystalline forms of the compound of formula (I) of the present invention may also be formulated into dosage forms for topical administration, including, for example, ointments, powders, suppositories, drops, sprays, and inhalants. Any one of the above salts or crystalline forms of the compound of formula (I) of the present invention as the active ingredient is mixed with a carrier acceptable under sterile and physiological conditions and optional preservatives, buffers or propellants that may be required if necessary.

The present invention also provides a pharmaceutical composition comprising any one of the above salts or crystalline forms of the compound of formula (I) of the present invention and a pharmaceutically acceptable carrier, excipient or diluent. In preparing the pharmaceutical composition, any one of the above salts or crystalline forms of the compound of formula (I) of the present invention is usually mixed with a pharmaceutically acceptable carrier, excipient or diluent.

The compositions of the present invention can be formulated into conventional pharmaceutical preparations according to conventional preparation methods. The pharmaceutical preparations are, for example, tablets, pills, capsules, powders, granules, emulsions, suspensions, dispersions, solutions, syrups, ointments, drops, suppositories, inhalants, and sprays.

Any one of the above salts or crystalline forms of the compound of formula (I) of the present invention can be administered alone or in combination with other pharmaceutically acceptable therapeutic agents, especially in combination with other antitumor drugs. Such therapeutic agents include, but are not limited to, for example, alkylating agents (e.g., cisplatin, oxaliplatin, carboplatin, cyclophosphamide, chlormethine, melphalan, chlorambucil, busulfan, temozolomide, and nitrosourea); antimetabolites (e.g., gemcitabine and antifolates such as fluoropyrimidines (e.g., 5-fluorouracil and tegafur), raltitrexed, methotrexate, cytarabine, and hydroxyurea); antitumor antibiotics (e.g., anthracyclines such as adriamycin, bleomycin, doxorubicin, daunorubicin, epirubicin, idarubicin, mitomycin C, actinomycin, and mithramycin); antimitotic agents (e.g., vinca alkaloids, such as vincristine, vinblastine, vindesine, vinorelbine; and taxanes, such as paclitaxel and taxotere); topoisomerase inhibitors (e.g., epipodophyllotoxins (e.g., etoposide and teniposide), amsacrine, topotecan, and camptothecin) and the like. The ingredients to be combined may be administered simultaneously or sequentially, in a single preparation or in separate preparations. The combination includes not only the combination of any one of the above salts or crystalline forms of the compound of formula (I) of the present invention and one other active agent, but also includes any one of the above salts or crystalline forms of the compound of formula (I) of the present invention and a combination of two or more other active agents.

The present application will be illustrated by the following examples.

### EXAMPLES

The present invention will be further described below in conjunction with the examples, and it should be understood that the examples are only used to further illustrate and elucidate the present invention, and are not intended to limit the present invention.

Unless otherwise defined, the related technical and scientific terms in this specification have the same meaning as commonly understood by a person skilled in the art. Although methods and materials similar or identical to those described herein can be used in experiments or practical applications, the materials and methods are described herein below. In case of conflict, the present specification, including definitions therein, will control, and in addition, the materials, methods and examples are not intended in a limiting sense, and are provided solely for the purpose of illustration. The present invention is further described below in conjunction with specific examples, which are not intended to limit the scope of the present invention.

The following experimental conditions were used in the examples:

X-ray powder diffractometer (XRPD)

| Instrument | Bruker D8 Advance |
|---|---|
| Detector | LYNXEYE_XE_T (1D model) |
| Opening angle | 2.94° |
| Scanning mode | Continuous PSD fast mode |
| Radiation source | Cu/K-Alpha1 (λ = 1.5418 Å) |
| X-ray source power | 40 kV, 40 mA |
| Step size | 0.02° |
| Time per step | 0.06 seconds/step |
| Scanning range | 3° to 40° |
| Slit in primary | Twin_Secondary Motorized slit 10.0 mm |
| beam path | according to sample length; |
| | SollerMount axial Soller angle 2.5° |
| Slit in secondary | Detector OpticsMount Soller slit 2.5°; |
| beam path | Twin_Secondary Motorized slit 5.2 mm |
| Rotation speed | 15 rpm |
| of sample | |
| XRPD plate | monocrystalline silicon wafer, flat plate |
| or | |
| Instrument | Bruker D8 Advance |
| Detector | LYNXEYE_XE_T (1D model) |
| Opening angle | 2.94° |
| Scanning mode | Continuous PSD fast mode |
| Radiation source | Cu/K-Alpha1 (λ = 1.5418 Å) |
| X-ray source power | 40 kV, 40 mA |
| Step size | 0.02° |
| Time per step | 0.12 seconds/step |
| Scanning range | 3° to 40° |
| Slit in primary | Twin_Secondary Motorized slit 10.0 mm |
| beam path | according to sample length; |
| | SollerMount axial Soller angle 2.5° |
| Slit in secondary | Detector OpticsMount Soller slit 2.5°; |
| beam path | Twin_Secondary Motorized slit 5.2 mm |
| Rotation speed | 15 rpm |
| of sample | |
| XRPD plate | monocrystalline silicon wafer, flat plate |

Differential scanning calorimetry (DSC)

| | |
|---|---|
| Instrument | TA Discovery 2500 or Q2000 |
| Sample pan | Tzero pan and Tzero sealing cap with pinhole |
| Temperature range | 30 to 250° C. or before degradation |
| Rate of | 10° C./min or 2° C./min |
| temperature rise | |
| Flow rate of nitrogen | 50 mL/min |
| Sample amount | about 1 to 2 mg |

Thermogravimetic analysis (TGA)

| | |
|---|---|
| Instrument | Discovery 5500 or Q5000 |
| Sample pan | aluminum pan, open |
| Flow rate | 10 mL/min for balance; 25 mL/min for sample |
| of nitrogen | |
| Initial temperature | Environmental condition (lower than 35° C.) |
| Final temperature | 300° C. or abort next segment if weight <80%(w/w) |
| | (the weight loss of the compound is not greater than |
| | 20%(w/w)) |
| Rate of | 10° C./min |
| temperature rise | |
| Sample amount | about 2 to 10 mg |

Polarizing microscope (PLM)

| | |
|---|---|
| Instrument | Nikon LV100POL |
| Method | crossed polarizer, with the addition of silicone oil |

Magnetic resonance imaging (NMR)

| | |
|---|---|
| Instrument | Bruker Avance-AV 400M |
| Frequency | 400 MHz |
| Probe | 5 mm PABBO BB/19F-1H/D |
| | Z-GRD Z108618/0406 |
| Number of scans | 8 |
| Temperature | 297.6 K |
| Relaxation | 1 second |

High performance liquid chromatography (HPLC)

| Instrument | Shimadzu LC40 | | |
|---|---|---|---|
| Wavelength | 210 nm | | |
| Column | Waters Xbridge C18 3.5 μm, 4.6*150 mm | | |
| Detector | DAD | | |
| Column | 40° C. | | |
| temperature | | | |
| Flow rate | 1.0 mL/min | | |
| Mobile phase A | 20 mmol/L dipotassium hydrogen | | |
| | phosphate solution (adjusted | | |
| | with phosphoric acid to pH = 8.05) | | |
| Mobile phase B | acetonitrile | | |
| Diluent | acetonitrile/water (v:v = 1:1) | | |
| Injection volume | 5 μL | | |
| | Time (min) | Mobile phase A (%) | Mobile phase B (%) |
| Gradient | 0 | 70 | 30 |
| | 2 | 70 | 30 |
| | 27 | 45 | 55 |
| | 40 | 20 | 80 |
| | 45 | 20 | 80 |
| | 46 | 70 | 30 |
| | 56 | 70 | 30 |

Ion chromatography (IC)

| | |
|---|---|
| Instrument | Metrohm 940 professional IC |
| Sample center | 889 IC |
| Detector | conductivity detector |
| Eluent (anion) | 3.2 mmol/L Na₂CO₃ + 1.0 mmol/L NaHCO₃ |
| Suppressor | 0.5% H₂SO₄ |
| solution | |
| Column | Anion A SUPP 5-150 or Cation Column C4-150 |
| Column | 30° C. |
| temperature | |
| Flow rate | 0.7 mL/min (anion) |
| Injection volume | 20 μL |

The free base concentration and acid ion concentration in the same sample were determined by IC, and the base:acid stoichiometric ratio in this sample was then calculated as follows:

$$free base : acid ion = \frac{{C}_{F}}{{M}_{F}} : \frac{{C}_{C}}{{M}_{c}}$$

wherein C_{F} is the free base concentration (mg/mL), M_{F} is the molar mass of the free base (g/mol), C_{C} is the acid ion concentration (mg/mL), and M_{C} is the molar mass of the acid ion (g/mol).

### Example 1: Amorphous Free Base (Sample No. Y11526-45-RV-FWD1509-AF-SU12)

About 300 mg of the compound of formula (I) (with a purity of 99.9%) were weighed and placed in a 40 mL glass bottle, and 10 mL of methanol/acetonitrile (v:v=1:1) were added to obtain a clear solution. The clear solution was subjected to rapid volatilization (i.e., rotary evaporation) to remove the solvent to afford about 290 mg of an off-white solid in about 97% yield.

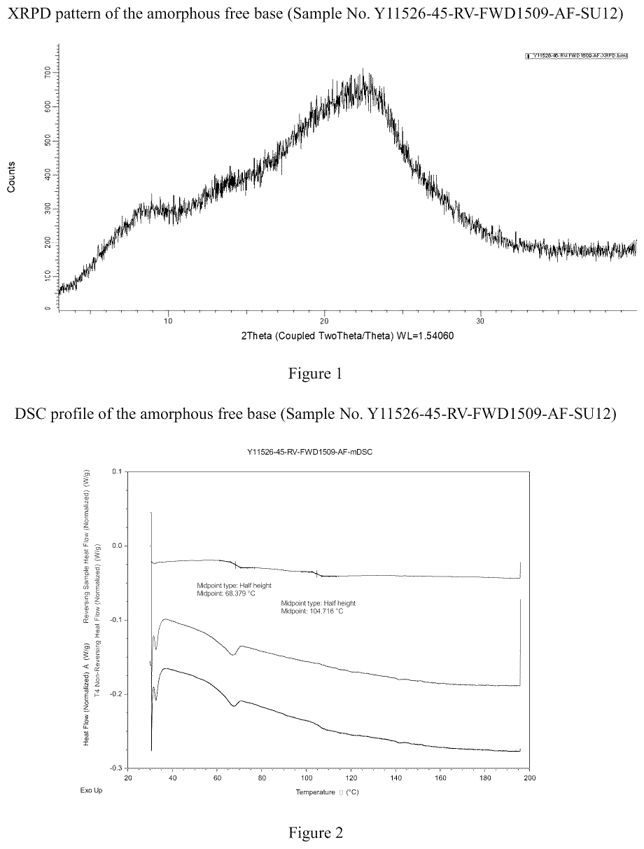

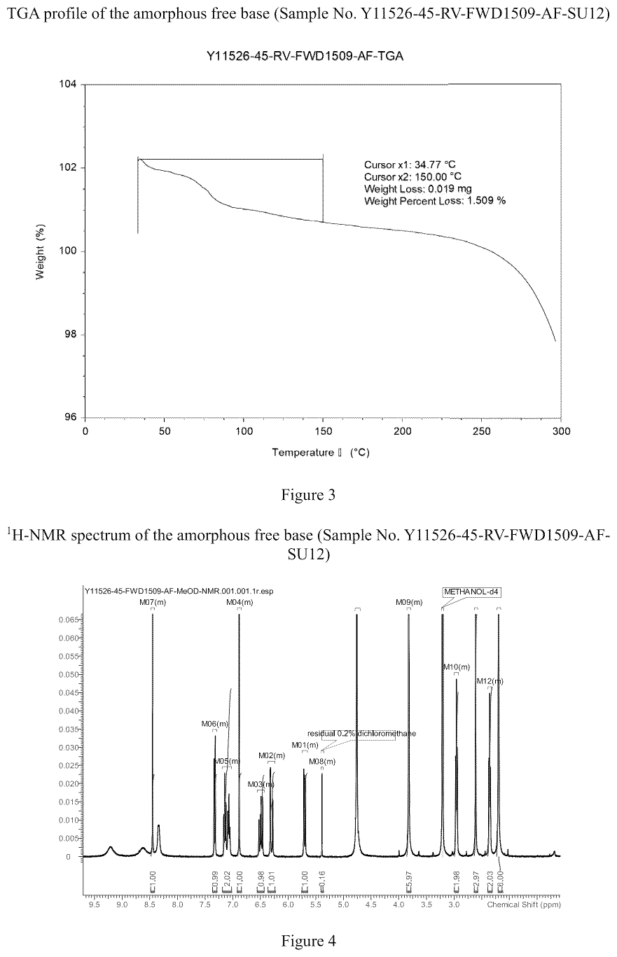

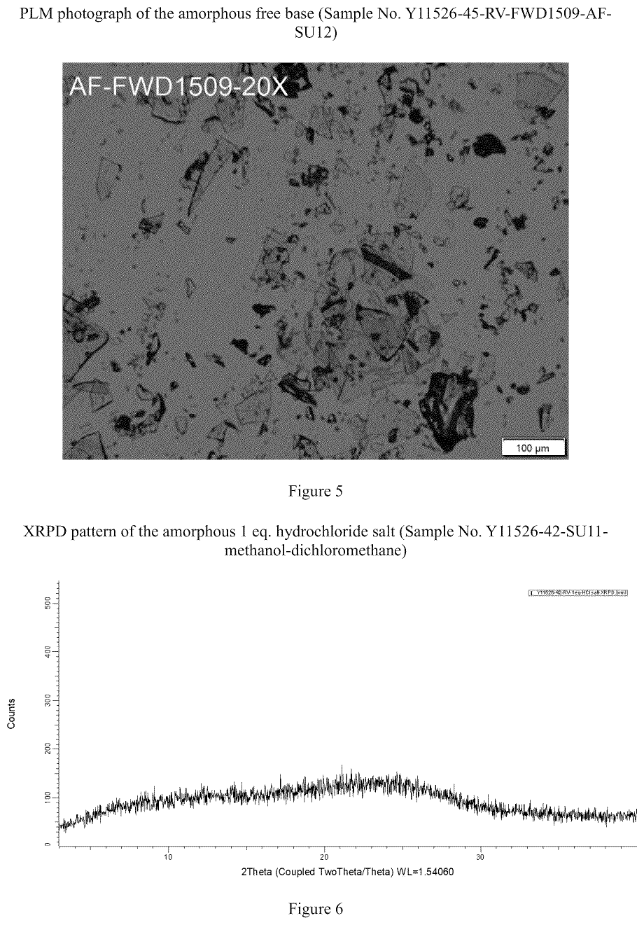

HPLC showed that the product had a purity of 99.9%. PLM showed that the product was an irregular sample (FIG. 5). XRPD showed that the product was amorphous (FIG. 1). DSC showed that the product had a glass-transition temperature of 68.4° C. and 104.7° C. (FIG. 2). TGA showed that the product had a weight loss of about 5.1% at 150° C. (FIG. 3).

The amorphous free base (Sample No. Y11526-45-RV-FWD1509-AF-SU12) was chemically and physically stable at the end of the preparation.

### Example 2: Amorphous 1 Eq. Hydrochloride Salt (Sample No. Y11526-42-SU11-Methanol-Dichloromethane)

About 300 mg of the compound of formula (I) (with a purity of 99.9%) were weighed and placed in a 40 mL glass bottle, 20 mL of methanol/dichloromethane (v:v=1:1) and 1 eq. of a diluted hydrochloric acid solution (518 μL, 44 mg/mL, in methanol/dichloromethane (v:v=1:1)) were added, and the reaction was performed at 50° C. for 2 h to obtain a clear solution. The clear solution was subjected to rapid volatilization to remove the solvent to afford about 306 mg of a pale yellow solid in about 95% yield.

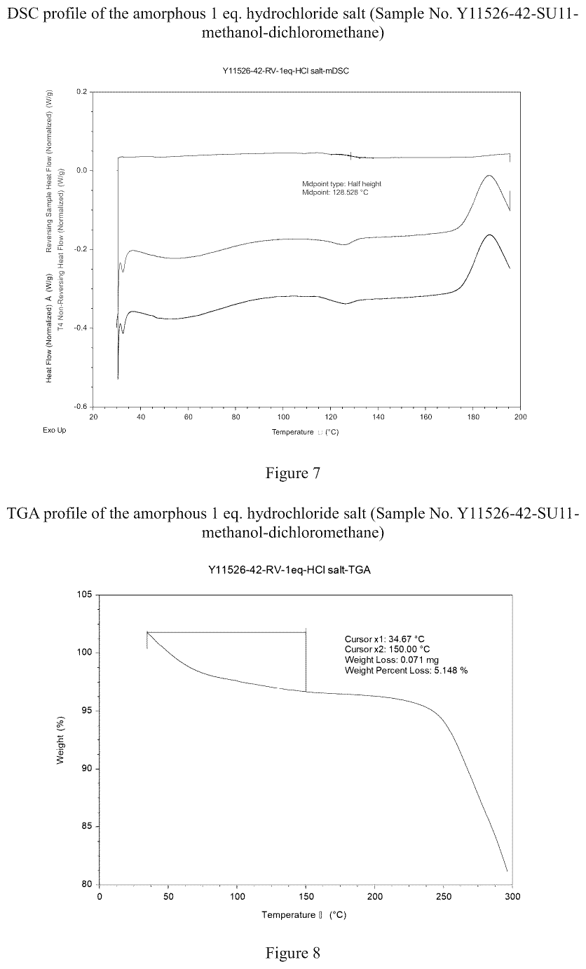

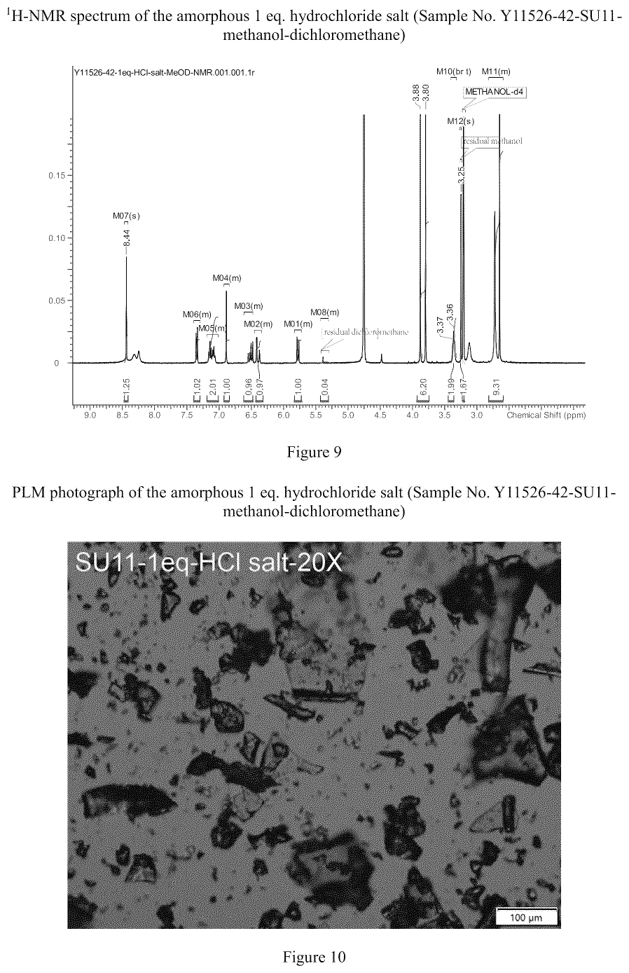

HPLC showed that the product had a purity of 99.9%. PLM showed that the product was an irregular sample (FIG. 10). XRPD showed that the product was amorphous (FIG. 6). DSC showed that the product had a glass-transition temperature of 128.5° C. (FIG. 7). TGA showed that the product had a weight loss of about 5.1% at 150° C. (FIG. 8). IC showed that the product had a free base concentration of 0.5 mg/mL and a chloride ion concentration of 33.7 mg/L, so the base:acid stoichiometric ratio in the product was about 1:1.

The amorphous 1 eq. hydrochloride salt (Sample No. Y11526-42-SU11-methanol-dichloromethane) was chemically and physically stable and reasonable in terms of the base:acid stoichiometric ratio at the end of the preparation.

### Example 3: Amorphous 2 Eq. Hydrochloride Salt (Sample No. Y11526-28-SU5-Methanol-Dichloromethane)

About 300 mg of the compound of formula (I) (with a purity of 99.9%) were weighed and placed in a 40 mL glass bottle, 5 mL of methanol/dichloromethane (v:v=1:1) and 2 eq. of a diluted hydrochloric acid solution (1010 μL, 44 mg/mL, in methanol/dichloromethane (v:v=1:1)) were added, and the reaction was performed at 50° C. for 2 h, then cooled down to 25° C. and continued at this temperature for about 2 h to obtain a clear solution. The resulting clear solution was subjected to rapid volatilization to remove the solvent to afford about 310 mg of a yellow solid in about 90% yield.

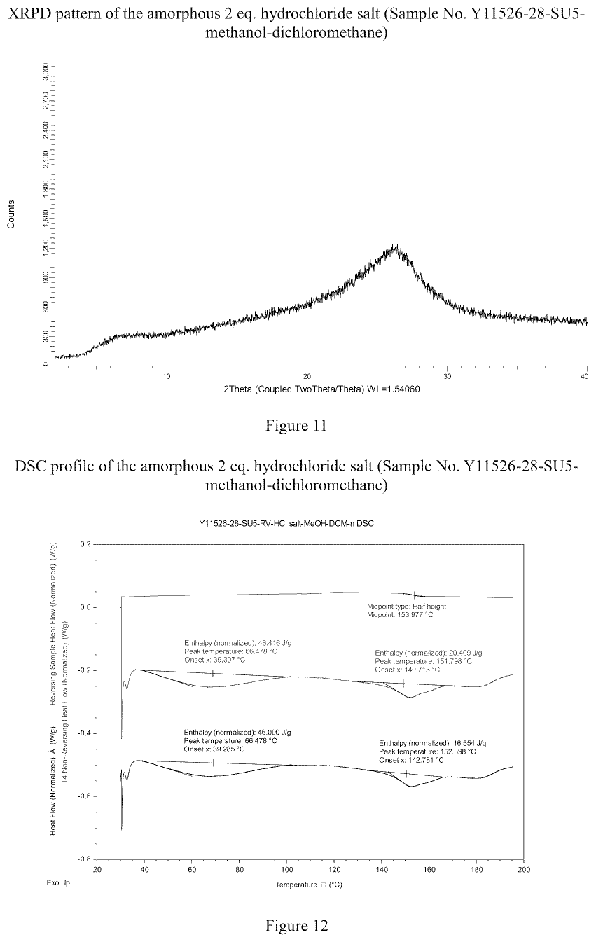

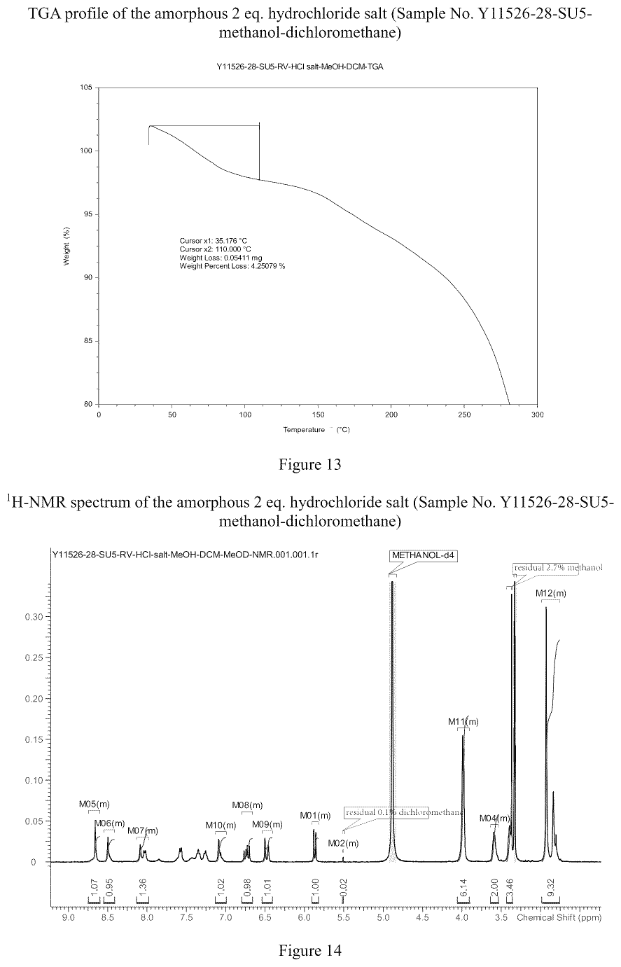

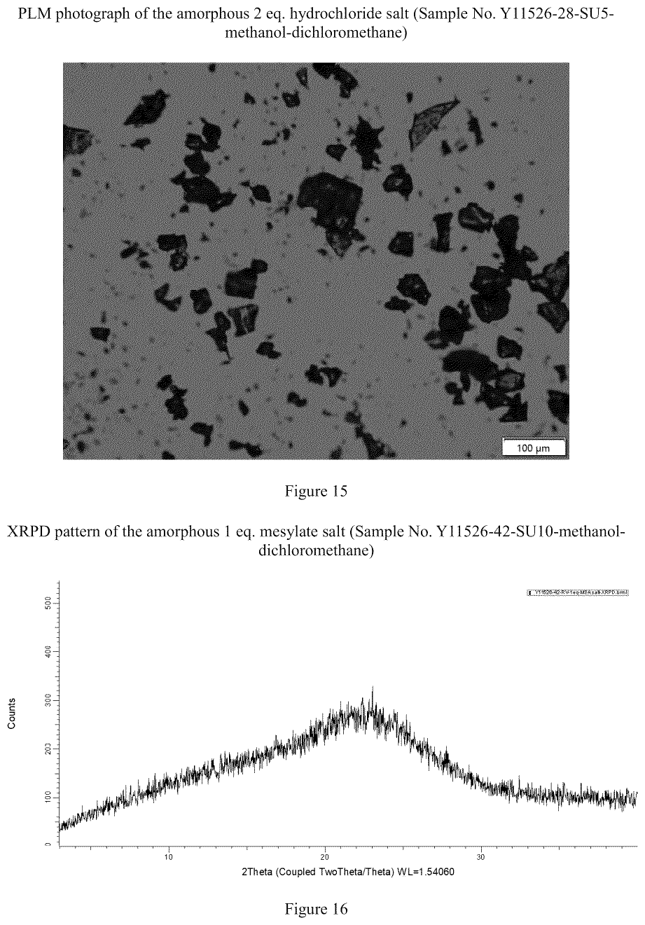

HPLC showed that the product had a purity of 99.9%. PLM showed that the product was an irregular sample (FIG. 15). XRPD showed that the product was amorphous (FIG. 11). DSC showed that the product had a glass-transition temperature of 154.0° C. (FIG. 12). TGA showed that the product had a weight loss of about 4.3% at 110° C. (FIG. 13). IC showed that the product had a free base concentration of 0.5 mg/mL and a chloride ion concentration of 77.4 mg/L, so the base:acid stoichiometric ratio in the product was about 1:2.

The amorphous 2 eq. hydrochloride salt (Sample No. Y11526-28-SU5-methanol-dichloromethane) was chemically and physically stable and reasonable in terms of the base:acid stoichiometric ratio at the end of the preparation.

### Example 4: Amorphous 1 Eq. Mesylate Salt (Sample No. Y11526-42-SU10-Methanol-Dichloromethane)

The weighed compound of formula (I) was added to acetone (11.70 times by weight with respect to that of the compound of formula (I) weighed) under stirring, and the temperature was increased to 45-55° C. After dissolving, the reaction mixture was filtered while hot, the filtrate was heated to 45-55° C., water (1 time by weight with respect to that of the compound of formula (I) weighed) was added, stirring was continued at 45-55° C., methanesulfonic acid (0.188 times by weight with respect to that of the compound of formula (I) weighed) was added dropwise within 30 min, and the mixture was stirred at 45-55° C. for 60±10 min. The reaction mixture was cooled down to 20-30° C. within 1.0-2.0 h, and crystallization was performed at 10-30° C. under stirring for 1.0-2.0 h. The reaction mixture was filtered, and the filter cake was rinsed twice with acetone (2*0.78 times by weight with respect to that of the compound of formula (I) weighed). The filter cake was dried to constant weight under the conditions of 40±5° C. and ≤−0.07 MPa to obtain a high-crystallinity material.

About 300 mg of the high-crystallinity material obtained were weighed, and about 50 mL of methanol/dichloromethane (v:v=1:1) were added to obtain a clear solution. The clear solution was subjected to rapid volatilization to remove the solvent to afford about 268 mg of a pale yellow solid in about 72% yield.

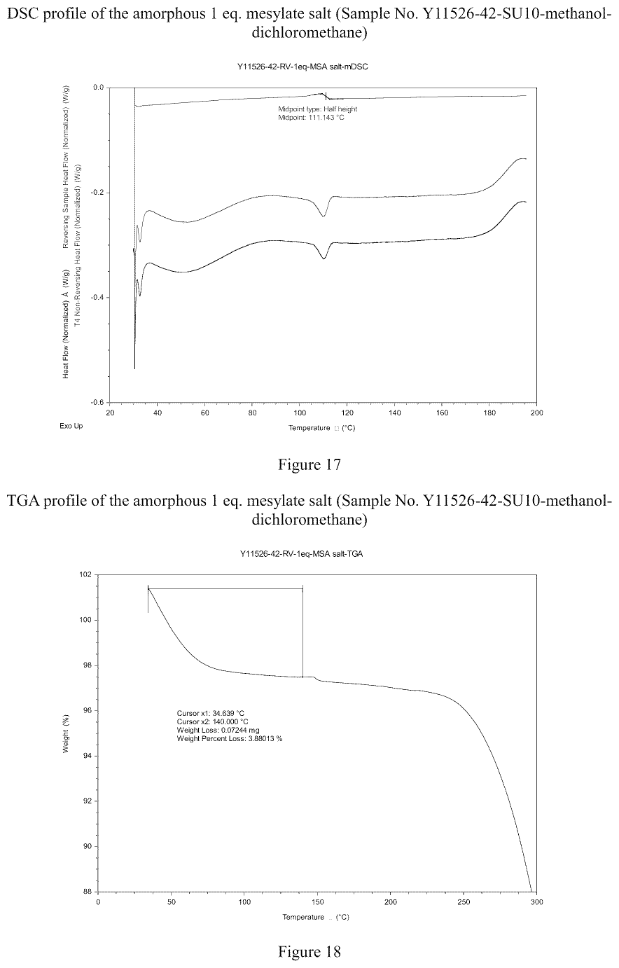

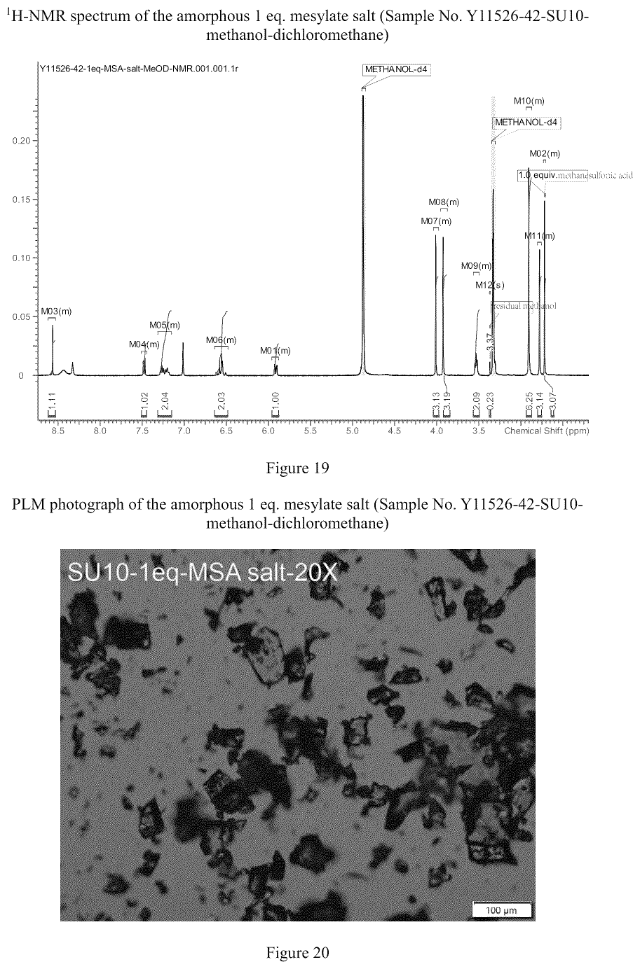

HPLC showed that the product had a purity of 99.9%. PLM showed that the product was an irregular sample (FIG. 20). XRPD showed that the product was amorphous (FIG. 16). DSC showed that the product had a glass-transition temperature of 111.1° C. (FIG. 17). TGA showed that the product had a weight loss of about 3.9% at 140° C. (FIG. 18). ¹H-NMR showed a base:acid stoichiometric ratio of about 1:1 in the product (FIG. 19).

The amorphous 1 eq. mesylate salt (Sample No. Y11526-42-SU10-methanol-dichloromethane) was chemically and physically stable and reasonable in terms of the base:acid stoichiometric ratio at the end of the preparation.

### Example 5: Amorphous 2 Eq. Mesylate Salt (Sample No. Y11526-28-SU4-Methanol-Dichloromethane)

About 300 mg of the compound of formula (I) (with a purity of 99.9%) were weighed and placed in a 40 mL glass bottle, 2 mL of methanol and 2 eq. of a diluted methanesulfonic acid solution (798 μL, 148 mg/mL, in methanol) were added, and the clear solution became a yellow opaque system. 2 mL of dichloromethane were added, the solution became clear, and 1 mL of methanol/dichloromethane (v:v=1:1) was continued to be added. The reaction was performed at 50° C. for 2 h, then cooled down to 25° C. and continued at this temperature for about 3 h to obtain a clear solution. The resulting clear solution was subjected to rapid volatilization to remove the solvent to afford about 380 mg of a yellow solid in about 85% yield.

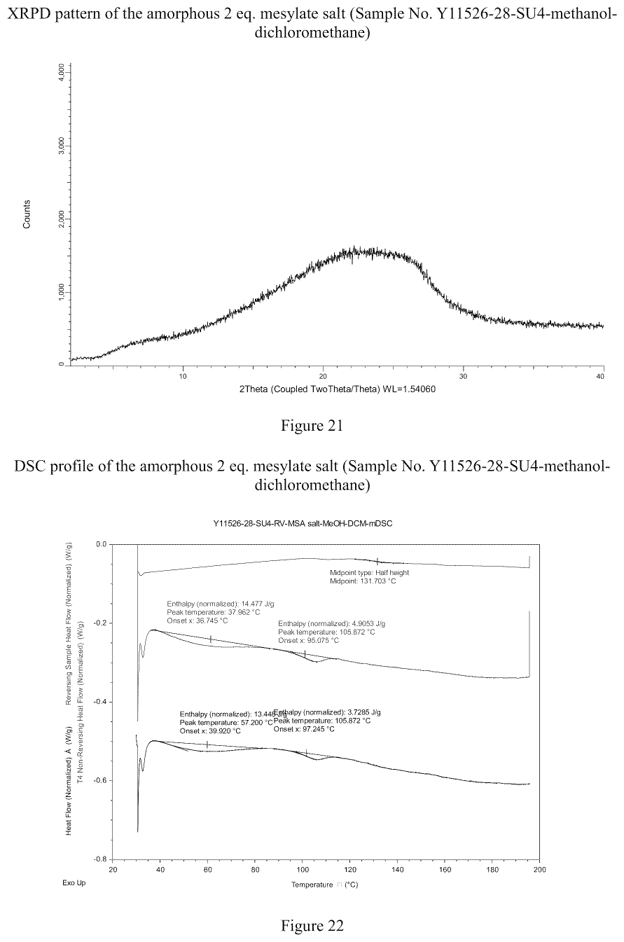

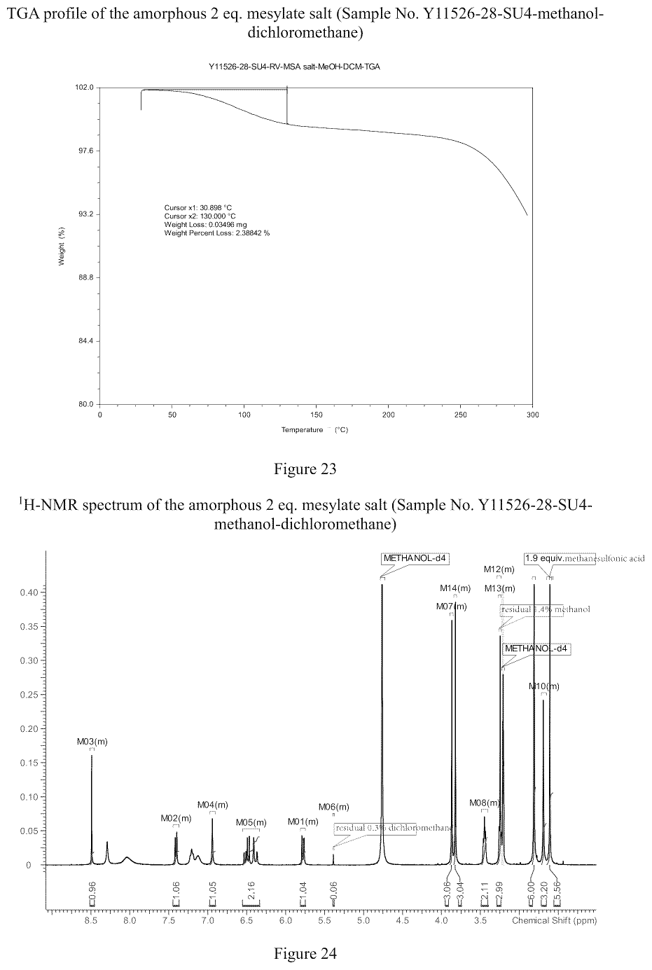

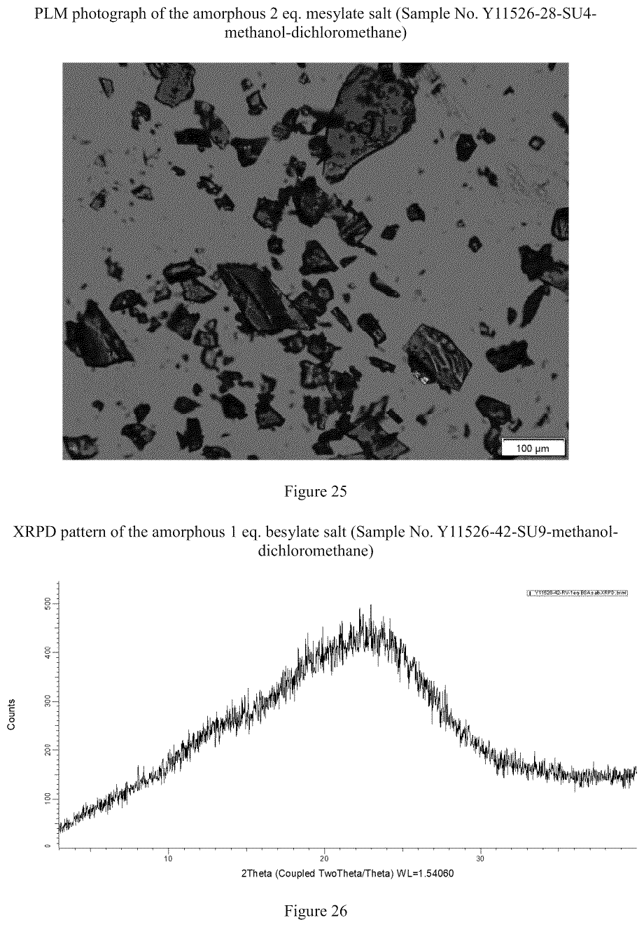

HPLC showed that the product had a purity of 99.9%. PLM showed that the product was an irregular sample (FIG. 25). XRPD showed that the product was amorphous (FIG. 21). DSC showed that the product had a glass-transition temperature of 131.7° C. (FIG. 22). TGA showed that the product had a weight loss of about 2.4% at 130° C. (FIG. 23). ¹H-NMR showed a base:acid stoichiometric ratio of about 1:2 in the product (FIG. 24).

The amorphous 2 eq. mesylate salt (Sample No. Y11526-28-SU4-methanol-dichloromethane) was chemically and physically stable and reasonable in terms of the base:acid stoichiometric ratio at the end of the preparation.

### Example 6: Amorphous 1 Eq. Besylate Salt (Sample No. Y11526-42-SU9-Methanol-Dichloromethane)

About 300 mg of the compound of formula (I) (with a purity of 99.9%) were weighed and placed in a 250 mL round-bottom flask together with 1 eq. of benzenesulfonic acid, 15 mL of water were added, and the reaction was performed at 50° C. for 2 h to obtain a clear solution followed by solid precipitation. The suspension was pre-frozen in a dry ice/ethanol mixture for 2 h, and then water was removed by freeze drying to afford a low-crystallinity sample. The freeze-dried sample was dissolved in 5 mL of methanol/dichloromethane (v:v=1:1) to obtain a clear solution. The clear solution was subjected to rapid volatilization to remove the solvent to afford about 320 mg of a pale yellow solid in about 83% yield.

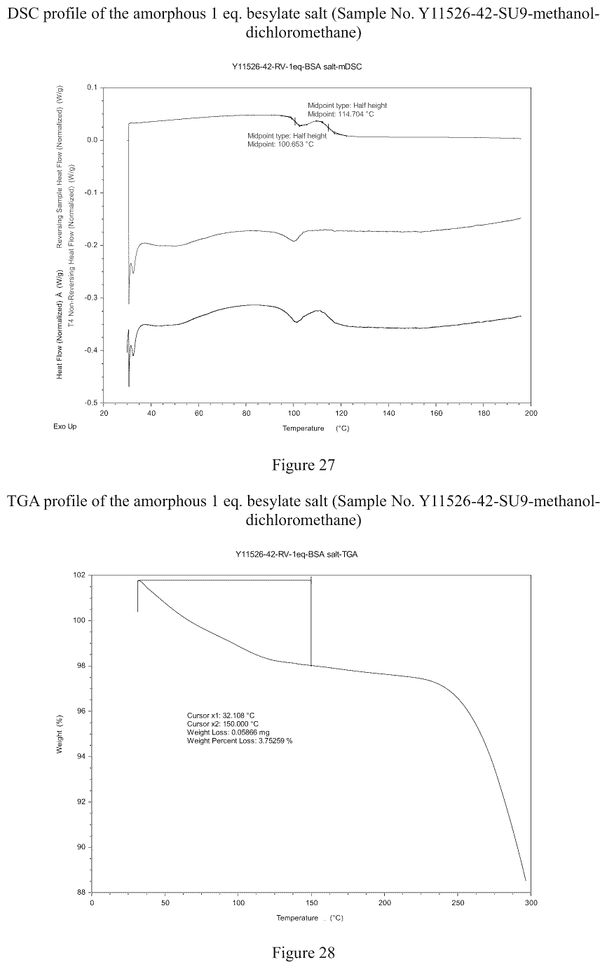

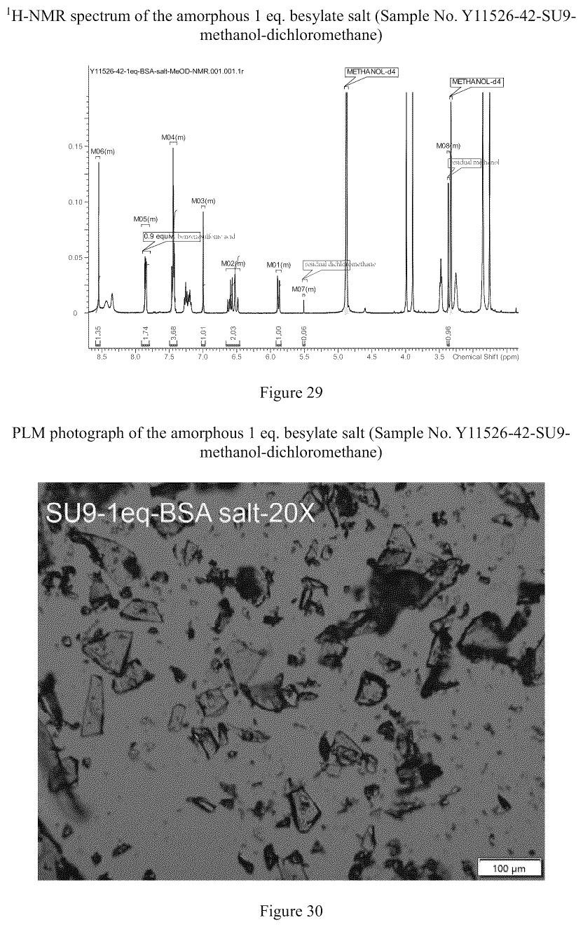

HPLC showed that the product had a purity of 99.7%. PLM showed that the product was an irregular sample (FIG. 30). XRPD showed that the product was amorphous (FIG. 26). DSC showed that the product had a glass-transition temperature of 100.7° C. and 114.7° C. (FIG. 27). TGA showed that the product had a weight loss of about 3.8% at 150° C. (FIG. 28). ¹H-NMR showed a base:acid stoichiometric ratio of about 1:1 in the product (FIG. 29).

The amorphous 1 eq. besylate salt (Sample No. Y11526-42-SU9-methanol-dichloromethane) was chemically and physically stable and reasonable in terms of the base:acid stoichiometric ratio at the end of the preparation.

### Example 7: Amorphous 2 Eq. Besylate Salt (Sample No. Y11526-30-SU1-Water)

About 300 mg of the compound of formula (I) (with a purity of 99.9%) were weighed and placed in a 250 mL round-bottom flask together with 2 eq. of benzenesulfonic acid, 15 mL of water were added, and the reaction was performed at 50° C. for 2 h to obtain a clear solution. The resulting clear solution was pre-frozen in a dry ice/ethanol mixture for 2 h, and then water was removed by freeze drying to afford about 380 mg of a yellow solid in about 81% yield.

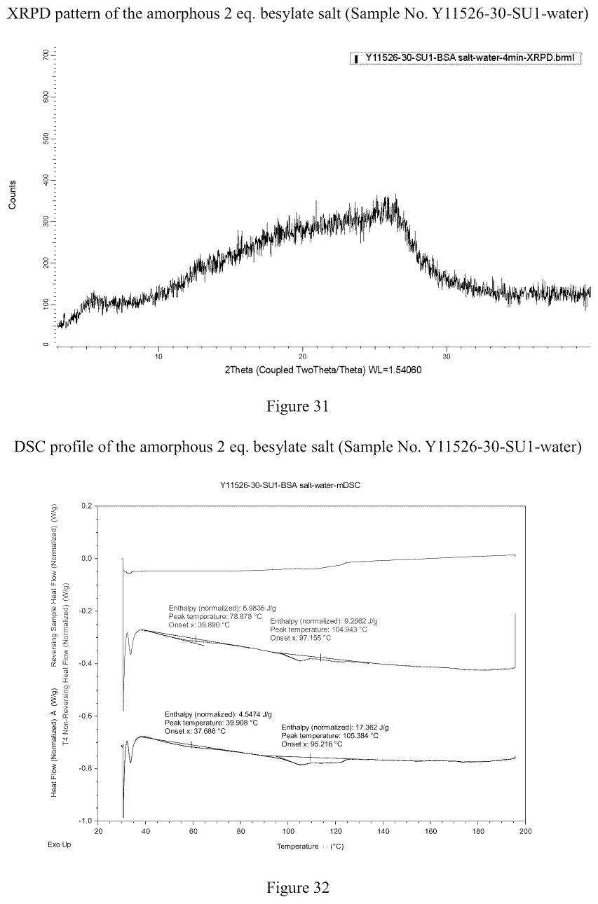

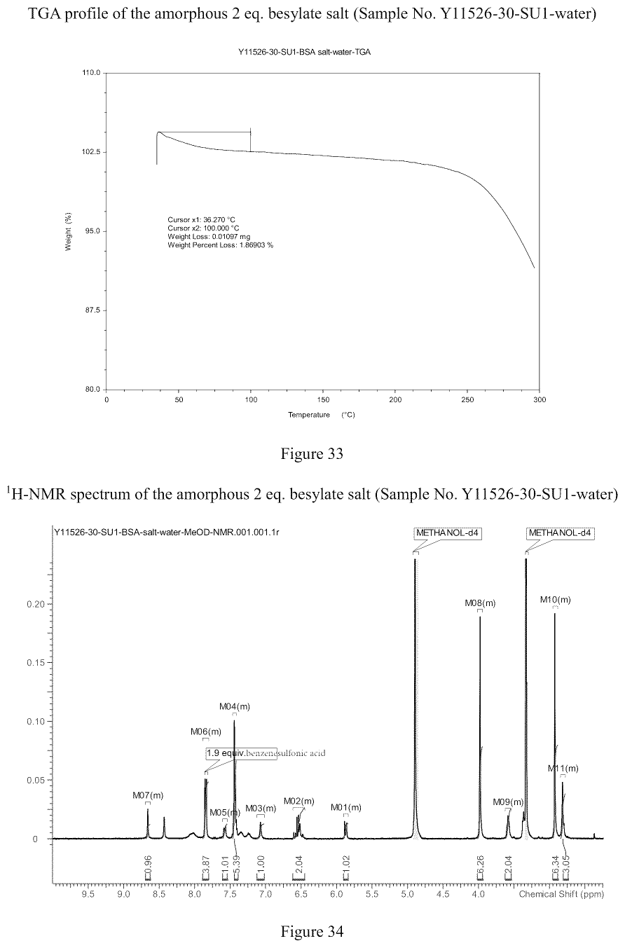

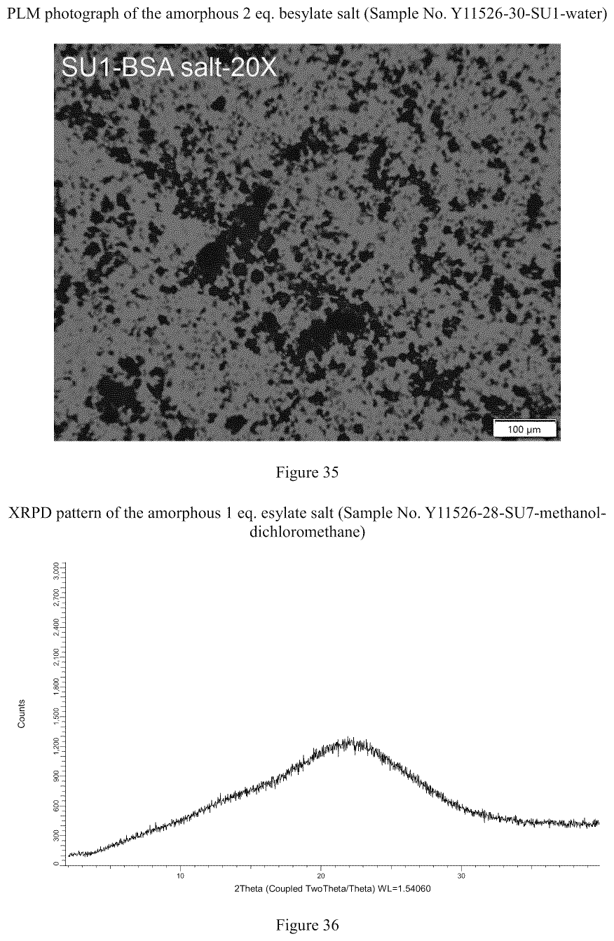

HPLC showed that the product had a purity of 99.7%. PLM showed that the product was an irregular sample (FIG. 35). XRPD showed that the product was amorphous (FIG. 31). DSC showed that the product had no glass-transition temperature (FIG. 32). TGA showed that the product had a weight loss of about 1.9% at 100° C. (FIG. 33). ¹H-NMR showed a base:acid stoichiometric ratio of about 1:2 in the product (FIG. 34).

The amorphous 2 eq. besylate salt (Sample No. Y11526-30-SU1-water) was chemically and physically stable and reasonable in terms of the base:acid stoichiometric ratio at the end of the preparation.

### Example 8: Amorphous 1 Eq. Esylate Salt (Sample No. Y11526-28-SU7-Methanol-Dichloromethane)

About 300 mg of the compound of formula (I) (with a purity of 99.9%) were weighed and placed in a 40 mL glass bottle, 5 mL of methanol/dichloromethane (v:v=1:1) and 1 eq. of a diluted ethanesulfonic acid solution (541 μL, 128 mg/mL, in methanol/dichloromethane (v:v=1:1)) were added, and the reaction was performed at 50° C. for 2 h, then cooled down to 25° C. and continued at this temperature for about 2 h to obtain a clear solution. The resulting clear solution was subjected to rapid volatilization to remove the solvent to afford about 340 mg of a yellow solid in about 90% yield.

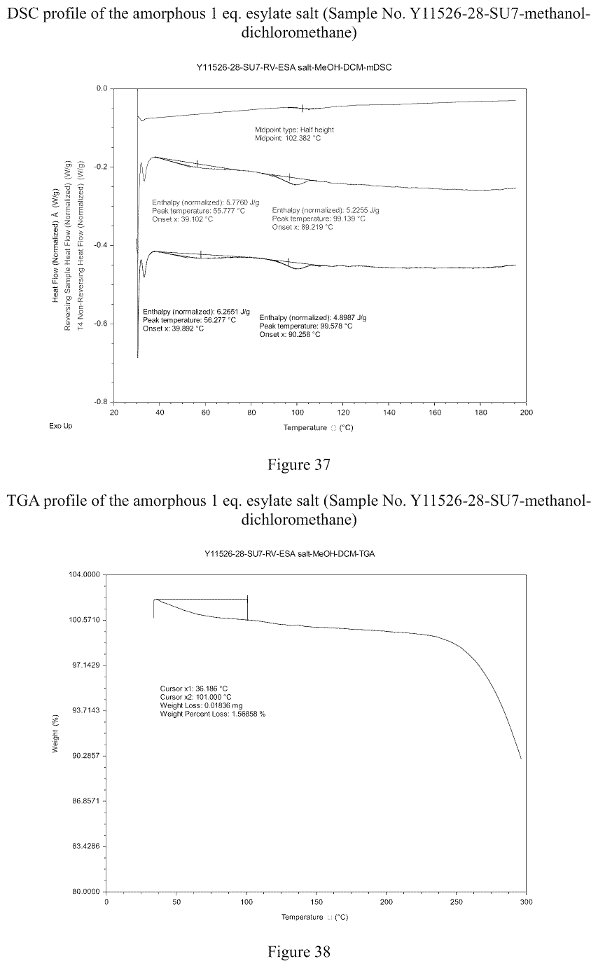

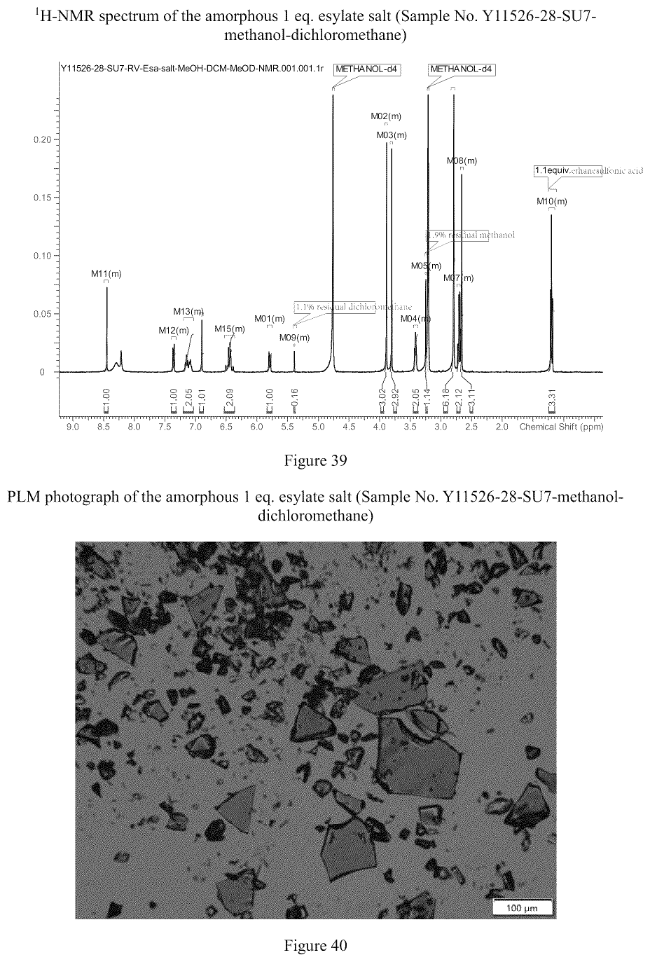

HPLC showed that the product had a purity of 99.9%. PLM showed that the product was an irregular sample (FIG. 40). XRPD showed that the product was amorphous (FIG. 36). DSC showed that the product had a glass-transition temperature of 102.4° C. (FIG. 37). TGA showed that the product had a weight loss of about 1.6% at 101° C. (FIG. 38). ¹H-NMR showed a base:acid stoichiometric ratio of about 1:1 in the product (FIG. 39).

The amorphous 1 eq. esylate salt (Sample No. Y11526-28-SU7-methanol-dichloromethane) was chemically and physically stable and reasonable in terms of the base:acid stoichiometric ratio at the end of the preparation.

### Example 9: Amorphous 1 Eq. Maleate Salt (Sample No. Y11526-30-SU2-Water)

About 300 mg of the compound of formula (I) (with a purity of 99.9%) were weighed and placed in a 250 mL round-bottom flask together with 1 eq. of maleic acid, 20 mL of water were added, and the reaction was performed at 50° C. for 2 h to obtain a clear solution. The resulting clear solution was pre-frozen in a dry ice/ethanol mixture for 2 h, and then water was removed by freeze drying to afford about 330 mg of a pale yellow solid in about 89% yield.

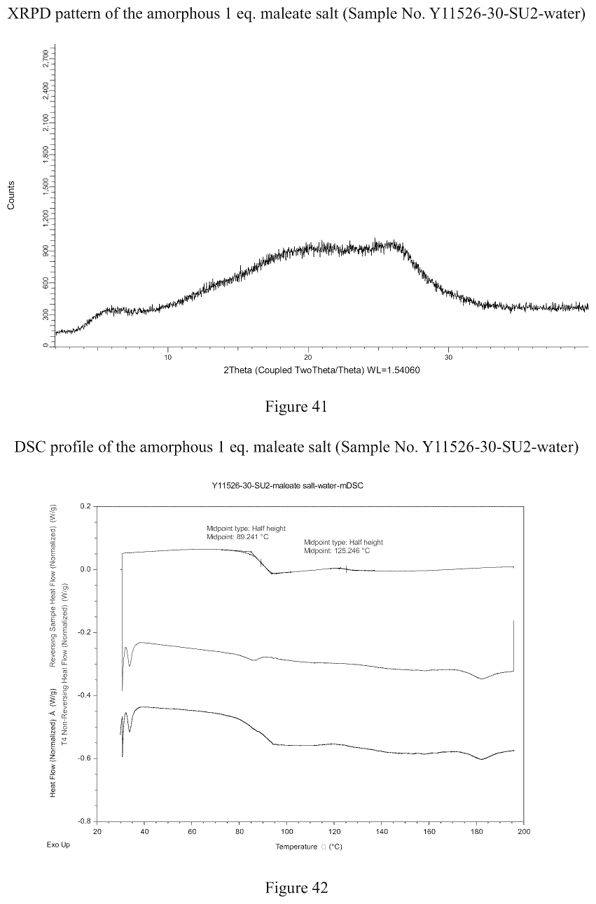

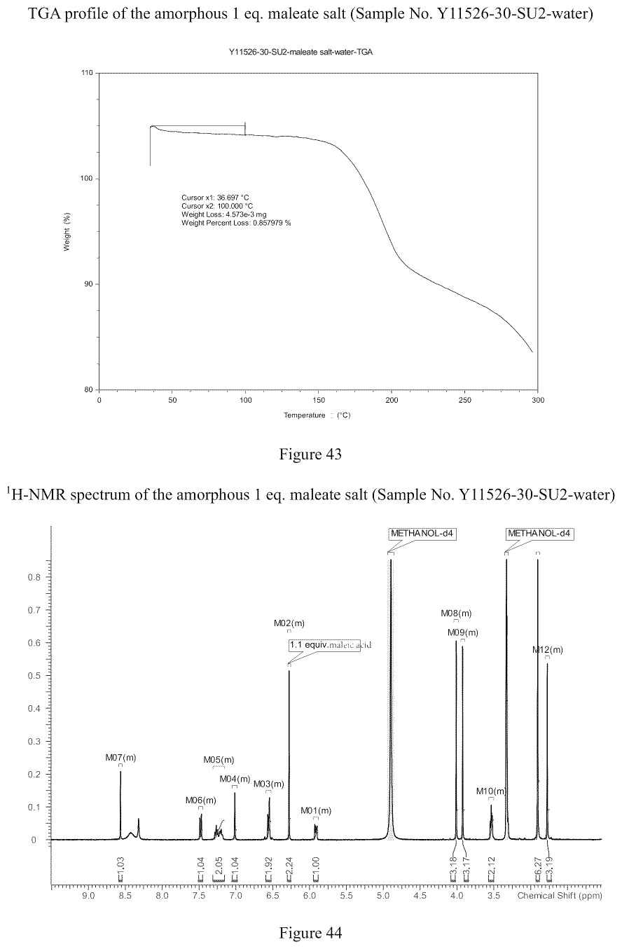

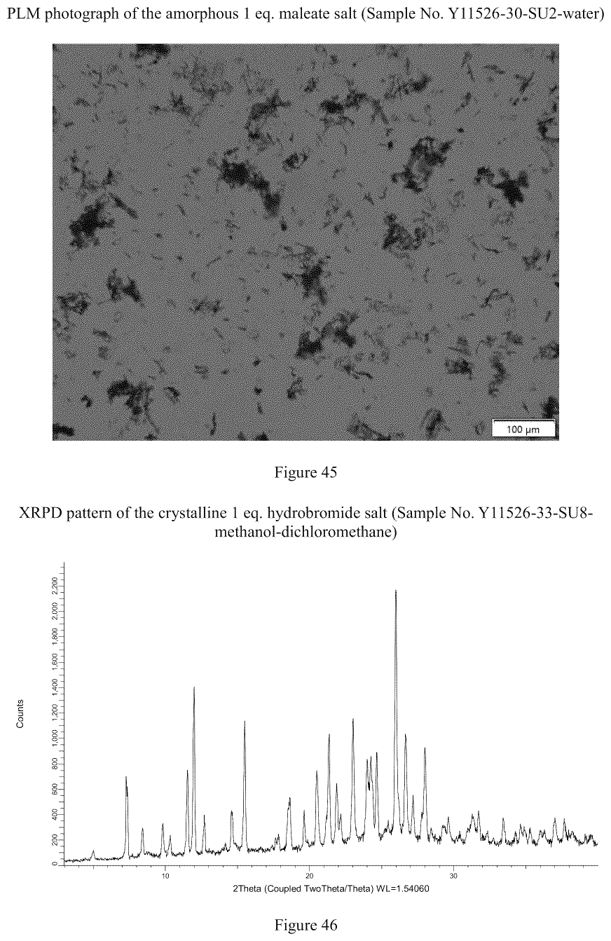

HPLC showed that the product had a purity of 99.9%. PLM showed that the product was an irregular sample (FIG. 45). XRPD showed that the product was amorphous (FIG. 41). DSC showed that the product had a glass-transition temperature of 89.2° C. and 125.2° C. (FIG. 42). TGA showed that the product had a weight loss of about 0.9% at 100° C. (FIG. 43). ¹H-NMR showed a base:acid stoichiometric ratio of about 1:1 in the product (FIG. 44).

The amorphous 1 eq. maleate salt (Sample No. Y11526-30-SU2-water) was chemically and physically stable and reasonable in terms of the base:acid stoichiometric ratio at the end of the preparation.

### Example 10: The Crystalline 1 Eq. Hydrobromide Salt (Sample No. Y11526-33-SU8-Methanol-Dichloromethane)

About 300 mg of the compound of formula (I) (with a purity of 99.9%) were weighed and placed in a 40 mL glass bottle, 20 mL of methanol/dichloromethane (v:v=1:1) and 1 eq. of a diluted hydrobromic acid solution (728 μL, 70 mg/mL, in methanol/dichloromethane (v:v=1:1)) were added, and the reaction was performed at 50° C. for 2 h to obtain a nearly clear solution (trace insoluble impurities). The impurities were filtered through a 0.45 μm filter membrane to obtain a clear solution, and the resulting clear solution was subjected to rapid volatilization to remove the solvent to afford about 310 mg of a pale yellow solid in about 90% yield.

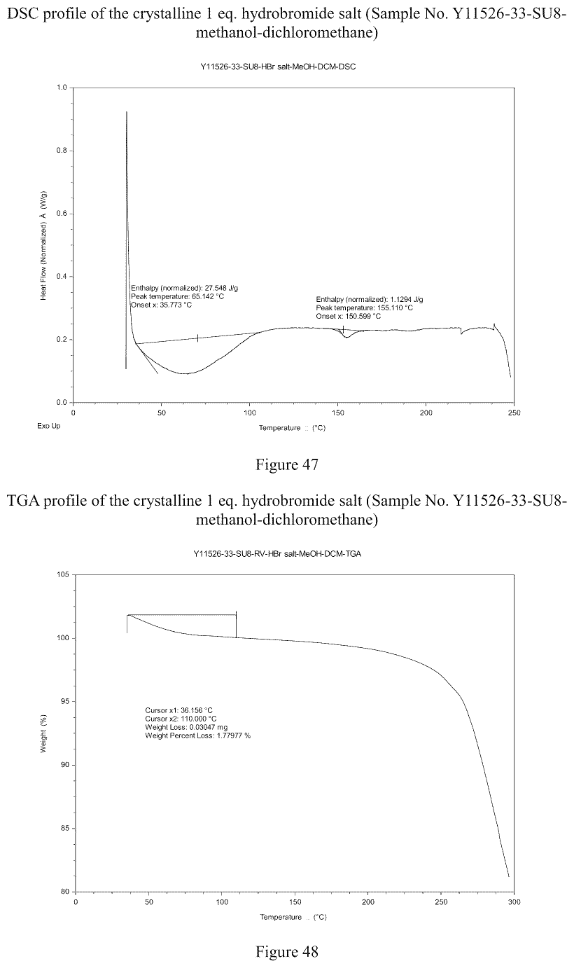

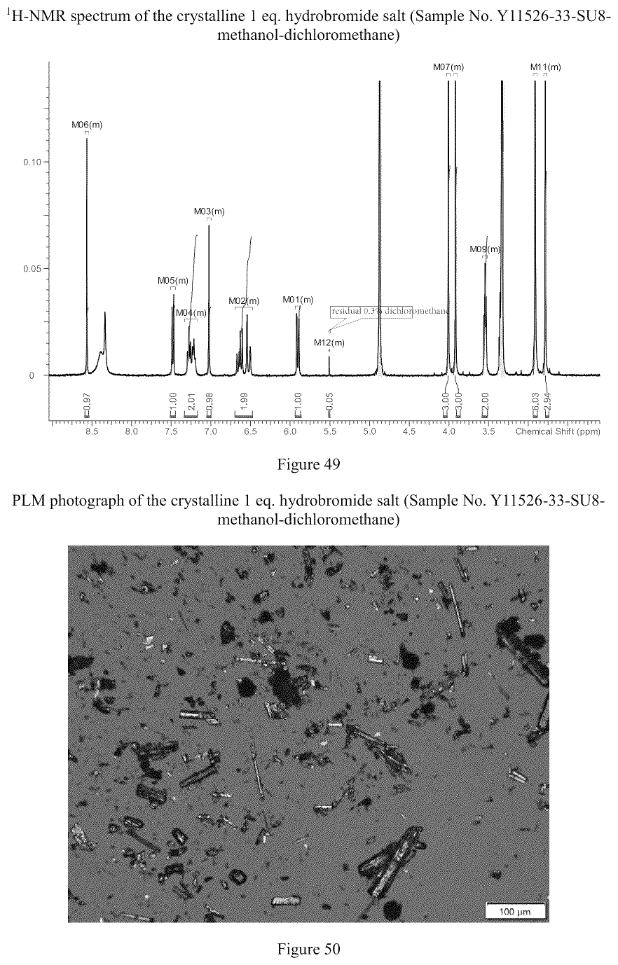

HPLC showed that the product had a purity of 99.9%. PLM showed that the product as rod-like and block-like samples (FIG. 50). XRPD showed that the product had a high crystallinity (FIG. 46). DSC showed that the product started to dehydrate from about 30° C. (FIG. 47). TGA showed that the product had a weight loss of about 1.8% at 110° C. (FIG. 48). KF showed that the product contained 1.9% of water. IC showed that the product had a free base concentration of 0.5 mg/mL and a bromide ion concentration of 68.1 mg/L, so the base:acid stoichiometric ratio in the product was about 1:1.

The crystalline 1 eq. hydrobromide salt (Sample No. Y11526-33-SU8-methanol-dichloromethane) was chemically and physically stable and reasonable in terms of the base:acid stoichiometric ratio at the end of the preparation.

### Example 11: Amorphous Nitrate Salt (Sample No. Y11526-18-RV7-Methanol-Dichloromethane)

About 100 mg of the compound of formula (I) (with a purity of 99.9%) were weighed and placed in a 40 mL glass bottle, 10 mL of 1,4-dioxane were added, 2 eq. of a diluted nitric acid solution (144 μL, 180 mg/mL, in 1,4-dioxane) were added, and the reaction was performed at 50° C. for 2 h. The resulting clear solution was pre-frozen in a dry ice/ethanol mixture for 2 h, and then 1,4-dioxane was removed by freeze drying to afford a nearly amorphous material.

About 80 mg of the nearly amorphous material were weighed and placed in a 40 mL glass bottle, and 10 mL of methanol/dichloromethane (v:v=1:1) were added to obtain a clear solution. The clear solution was subjected to rapid volatilization to remove the solvent to afford the amorphous nitrate salt.

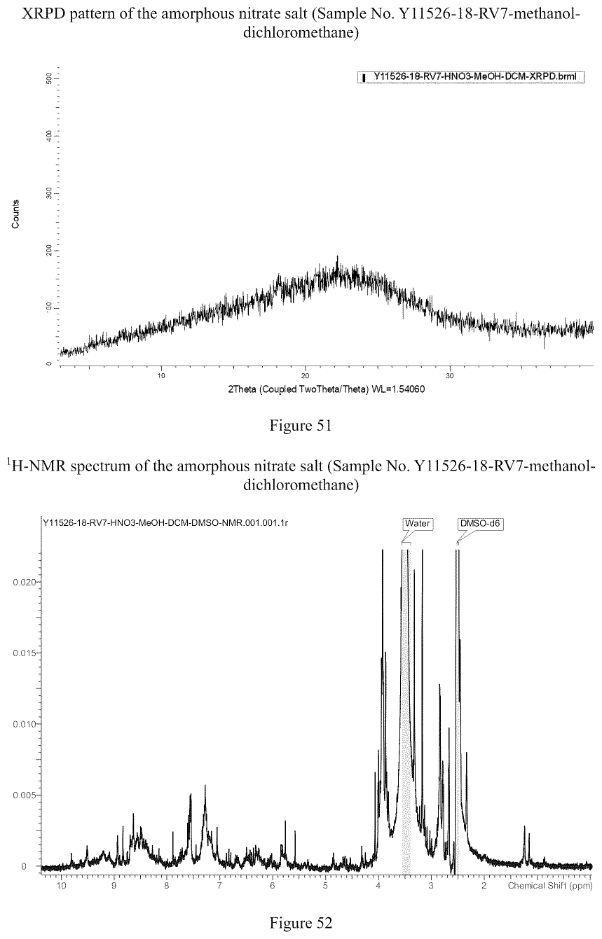

XRPD showed that the product was amorphous (FIG. 51). ¹H-NMR showed that the product was degraded (FIG. 52).

The amorphous nitrate salt (Sample No. Y11526-18-RV7-methanol-dichloromethane) was at least chemically unstable at the end of the preparation.

### Example 12: Amorphous Sulfate Salt (Sample No. Y11526-15-RV3-Methanol-Acetonitrile)

About 100 mg of the compound of formula (I) (with a purity of 99.9%) were weighed and placed in a 40 mL glass bottle, 10 mL of 1,4-dioxane were added, 2 eq. of a diluted sulfuric acid solution (228 μL, 180.3 mg/mL, in 1,4-dioxane) were added, and the reaction was performed at 50° C. for 2 h. The resulting clear solution was pre-frozen in a dry ice/ethanol mixture for 2 h, and then 1,4-dioxane was removed by freeze drying to afford a nearly amorphous material.

About 80 mg of the nearly amorphous material obtained were weighed and placed in a 40 mL glass bottle, and about 10 mL of methanol/acetonitrile (v:v=1:1) were added to obtain a clear solution. The clear solution was subjected to rapid volatilization to remove the solvent to afford the amorphous sulfate salt.

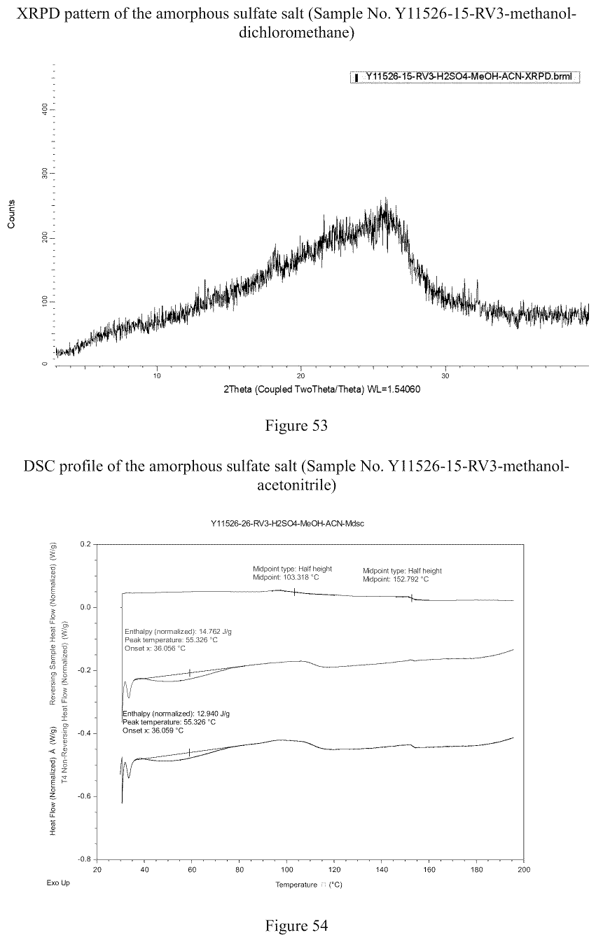

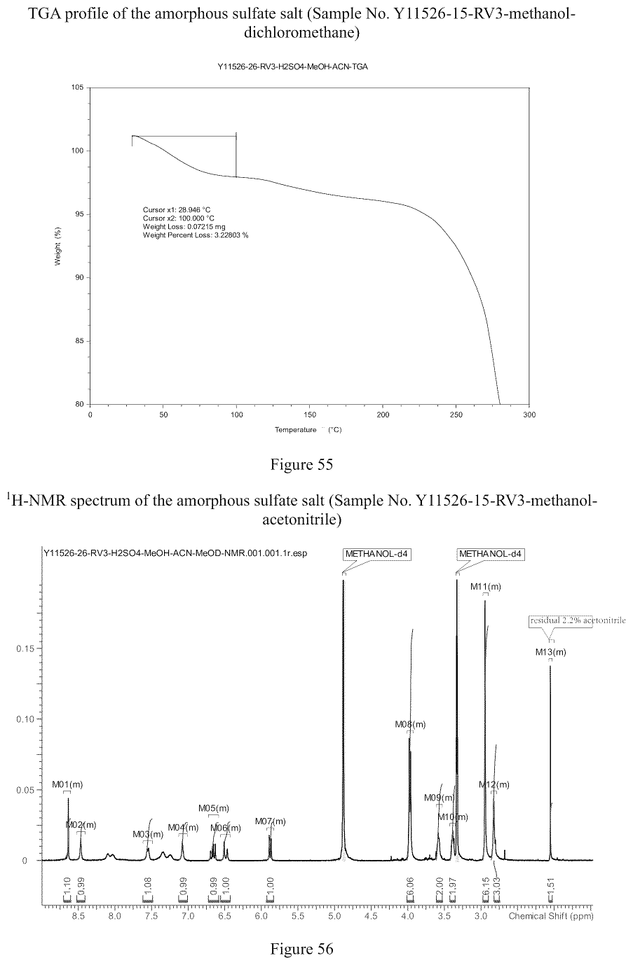

HPLC showed that the product had a purity of 99.7%. PLM showed that the product was an irregular sample (FIG. 57). XRPD showed that the product was amorphous (FIG. 53). DSC showed that the product had a glass-transition temperature of 103.3° C. and 152.8° C. (FIG. 54). TGA showed that the product had a weight loss of about 3.2% at 100° C. (FIG. 55). IC showed that the product had a free base concentration of 0.5 mg/mL and a sulfate concentration of 162.5 mg/L, so the base:acid stoichiometric ratio in the product was about 1:1.7, which was unreasonable.

The amorphous sulfate salt (Sample No. Y11526-15-RV3-methanol-acetonitrile) was chemically stable at the end of the preparation, but was physically unstable due to a certain hygroscopicity and unreasonable in terms of the alkali:acid stoichiometric ratio.

### Example 13: Amorphous 2 Eq. p-Tosylate Salt (Sample No. Y11526-23-FD11-Water)

About 100 mg of the compound of formula (I) (with a purity of 99.9%) were weighed and placed in a 40 mL glass bottle together with 2 eq. of p-toluenesulfonic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford a nearly amorphous material. The resulting nearly amorphous material was fully dissolved in 10 ml of water, and the resulting clear solution was pre-frozen in a dry ice/ethanol mixture for 2 h and then water was removed by freeze drying to afford the amorphous 2 eq. p-tosylate salt.

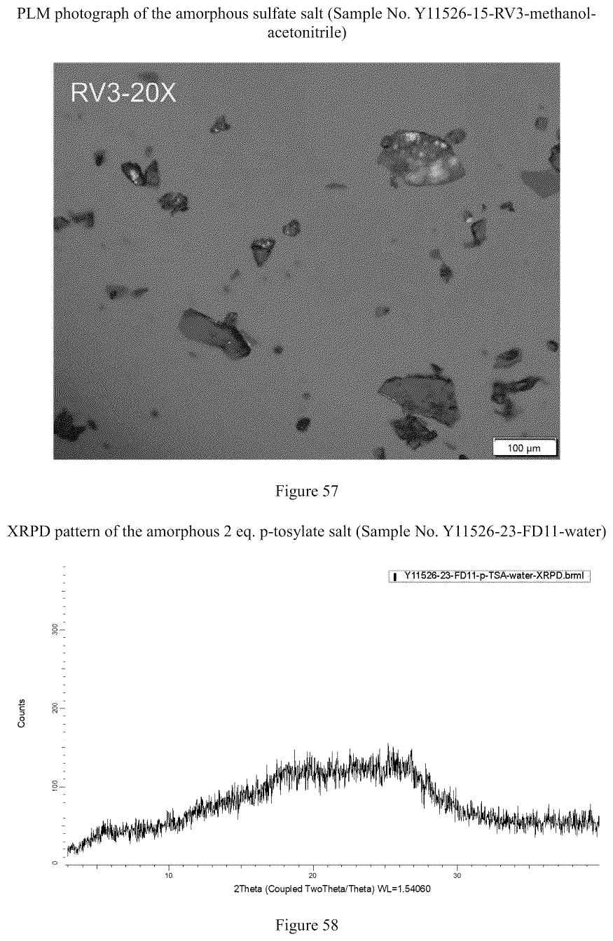

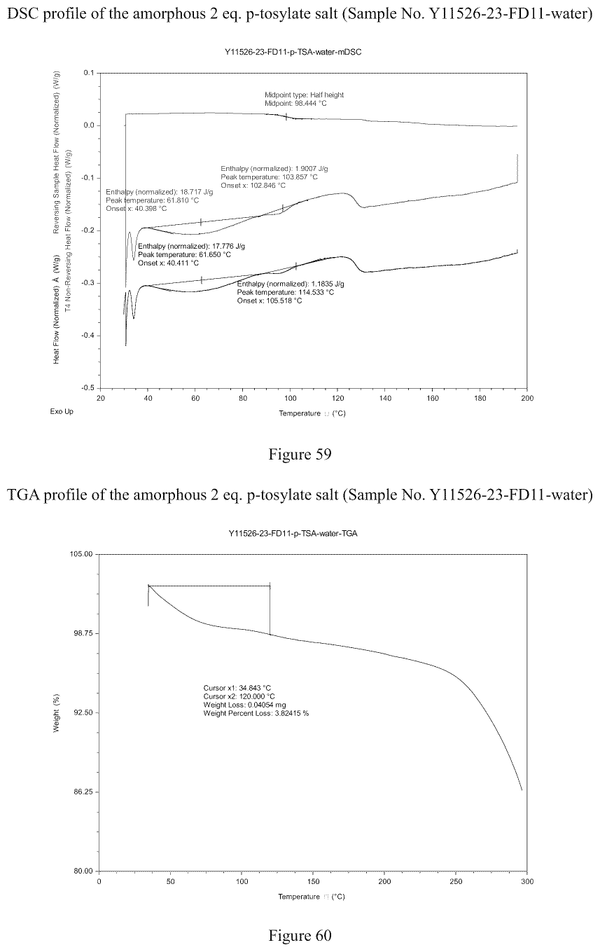

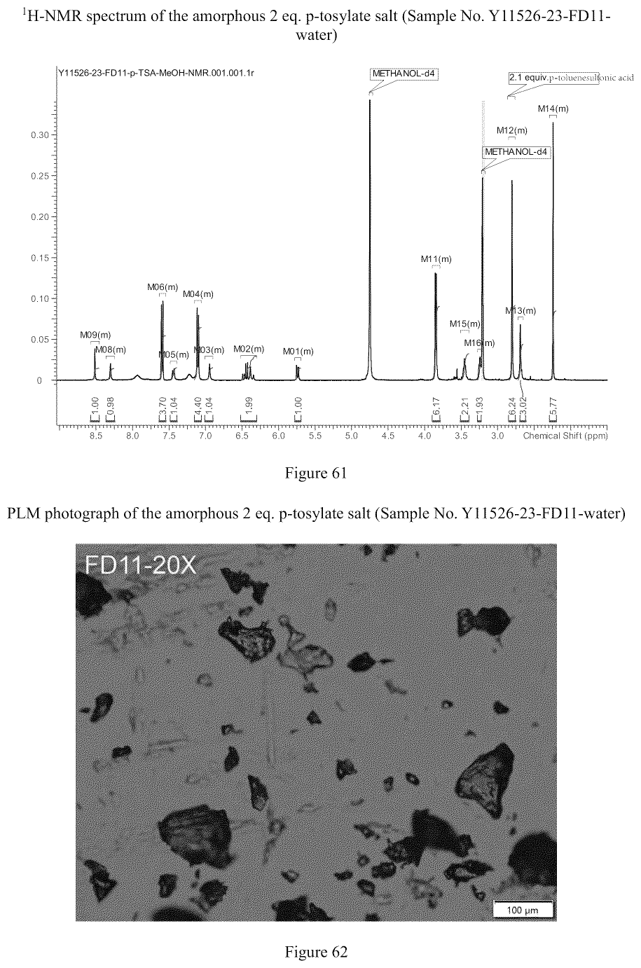

HPLC showed that the product had a purity of 99.6%. PLM showed that the product as a block-like sample (FIG. 62). XRPD showed that the product was amorphous (FIG. 58). DSC showed that the product had a glass-transition temperature of 98.4° C. (FIG. 59). TGA showed that the product had a weight loss of about 3.8% at 140° C. (FIG. 60). ¹H-NMR showed a base:acid stoichiometric ratio of about 1:2 in the product (FIG. 61).

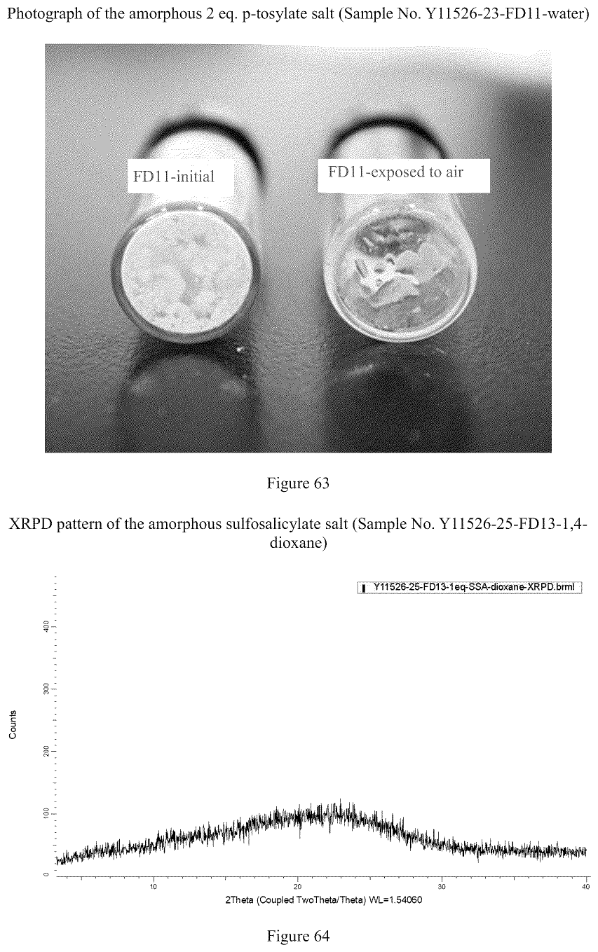

The amorphous 2 eq. p-tosylate salt (Sample No. Y11526-23-FD11-water) was chemically stable and reasonable in terms of the base:acid stoichiometric ratio at the end of the preparation, but was physically unstable due to caking and fusion to a glassy state upon short exposure to environmental conditions (FIG. 63).

### Example 14: Amorphous Sulfosalicylate Salt (Sample No. Y11526-25-FD13-1,4-Dioxane)

About 100 mg of the compound of formula (I) (with a purity of 99.9%) were weighed and placed in a 40 mL glass bottle together with 1 eq. of sulfosalicylic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous sulfosalicylate salt.

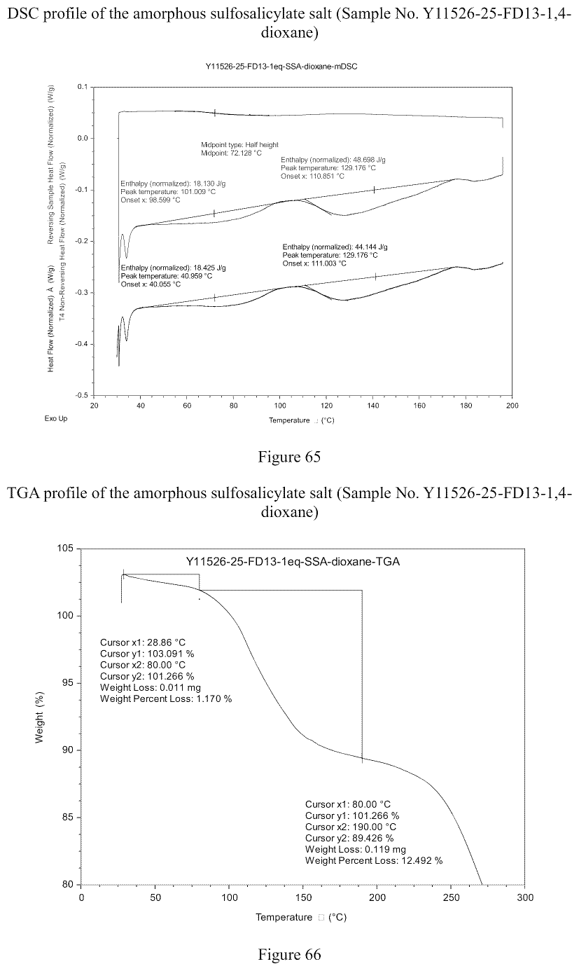

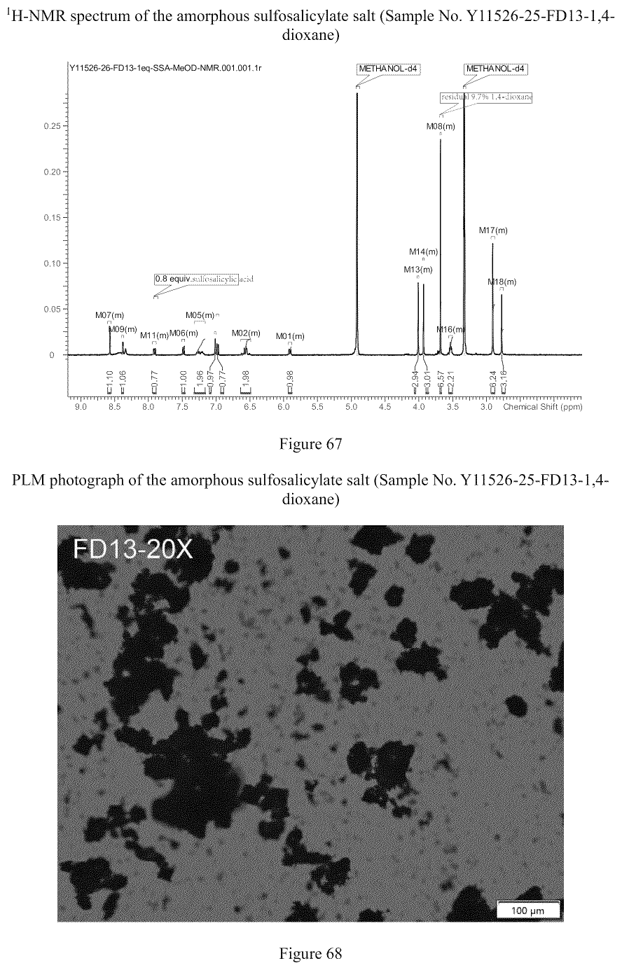

HPLC showed that the product had a purity of 99.7%. PLM showed that the product was an irregular sample (FIG. 68). XRPD showed that the product was amorphous (FIG. 64). DSC showed that the product had a glass-transition temperature of 72.1° C. (FIG. 65). TGA showed that the product had a weight loss of about 1.2% at 80° C. (FIG. 66). ¹H-NMR showed a base:acid stoichiometric ratio of about 1:0.8 in the product (FIG. 67), which was unreasonable.

The amorphous sulfosalicylate salt (Sample No. Y11526-25-FD13-1,4-dioxane) was chemically and physically stable, but unreasonable in terms of the base:acid stoichiometric ratio at the end of the preparation.

### Example 15: Amorphous Sulfosalicylate Salt (Sample No. Y11526-25-FD12-1,4-Dioxane)

About 100 mg of the compound of formula (I) (with a purity of 99.9%) were weighed and placed in a 40 mL glass bottle together with 2 eq. of sulfosalicylic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous sulfosalicylate salt.

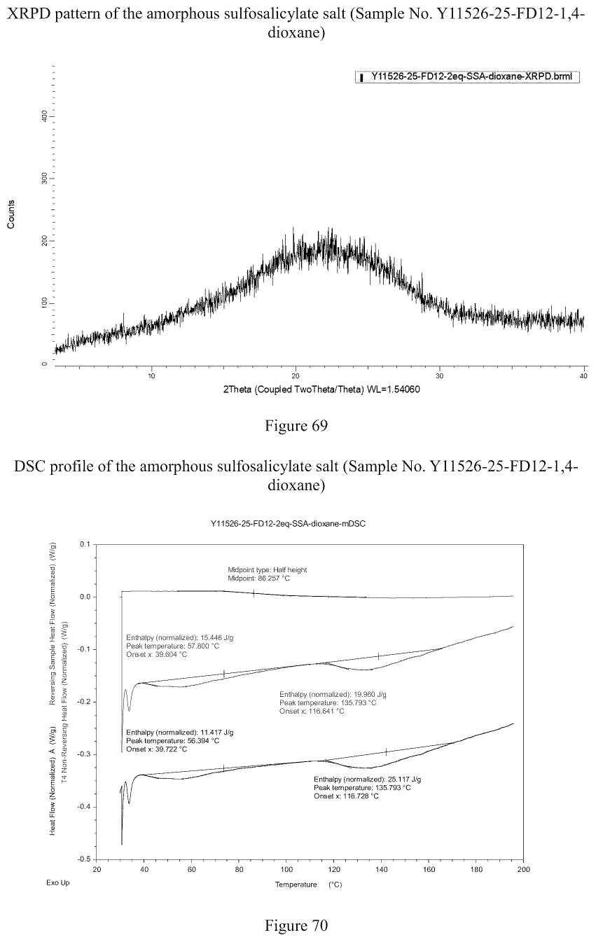

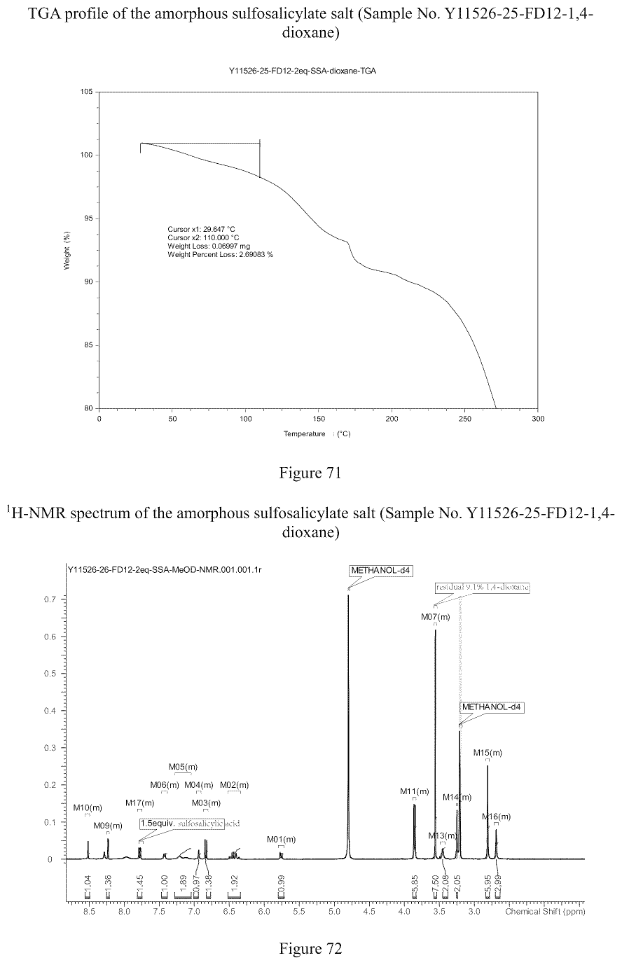

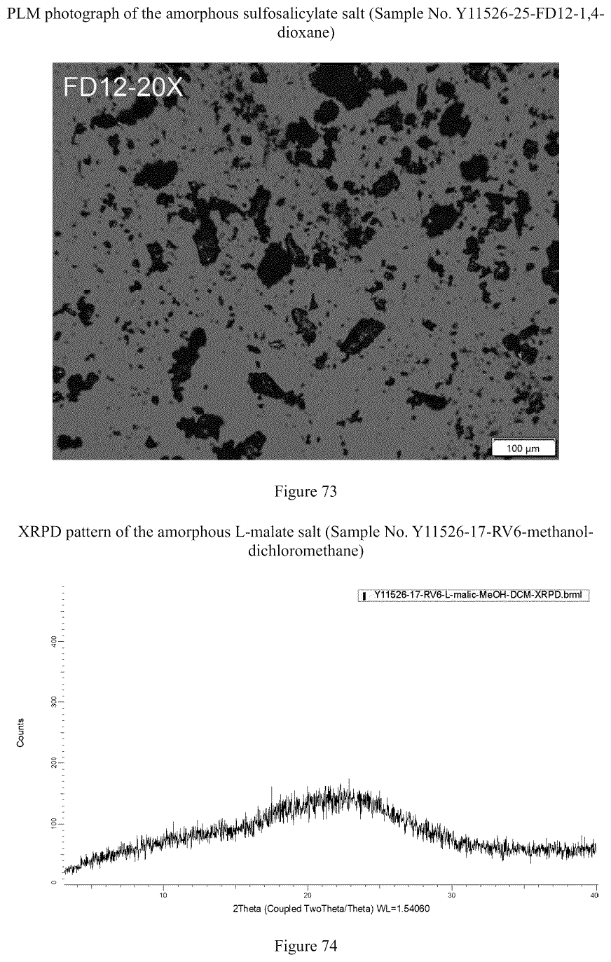

HPLC showed that the product had a purity of 99.8%. PLM showed that the product was an irregular sample (FIG. 73). XRPD showed that the product was amorphous (FIG. 69). DSC showed that the product had a glass-transition temperature of 86.3° C. (FIG. 70). TGA showed that the product had a weight loss of about 2.7% at 110° C. (FIG. 71). ¹H-NMR showed a base:acid stoichiometric ratio of about 1:1.5 in the product (FIG. 72), which was unreasonable.

The amorphous sulfosalicylate salt (Sample No. Y11526-25-FD12-1,4-dioxane) was chemically and physically stable, but unreasonable in terms of the base:acid stoichiometric ratio at the end of the preparation.

### Example 16: Amorphous L-Malate Salt (Sample No. Y11526-17-RV6-Methanol-Dichloromethane)

About 100 mg of the compound of formula (I) (with a purity of 99.9%) were weighed and placed in a 40 mL glass bottle together with 1 eq. of L-malic acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford a high-crystallinity material.

About 80 mg of the high-crystallinity material obtained were weighed and placed in a 40 mL glass bottle, and about 10 mL of methanol/dichloromethane (v:v=1:1) were added to obtain a clear solution. The clear solution was subjected to rapid volatilization to remove the solvent to afford the amorphous L-malate salt.

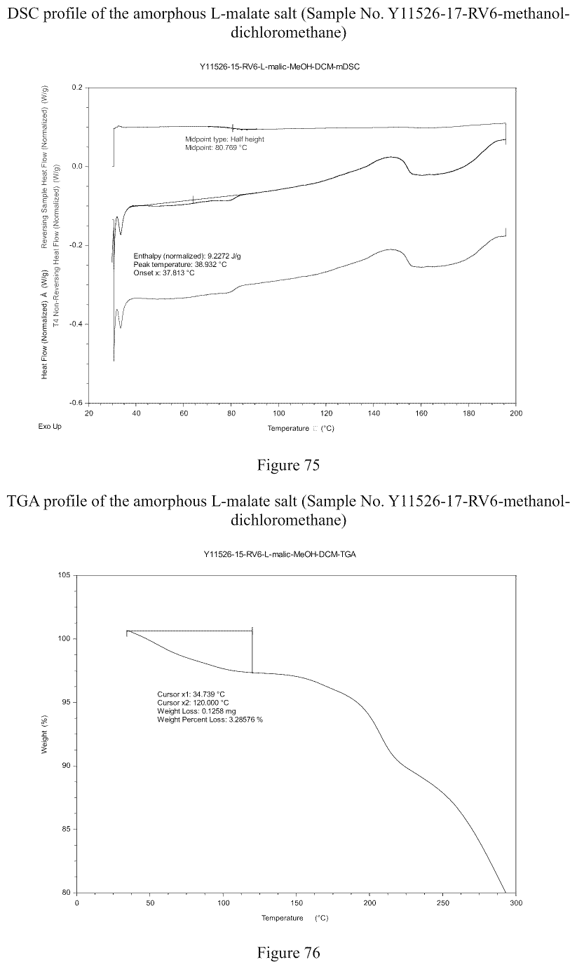

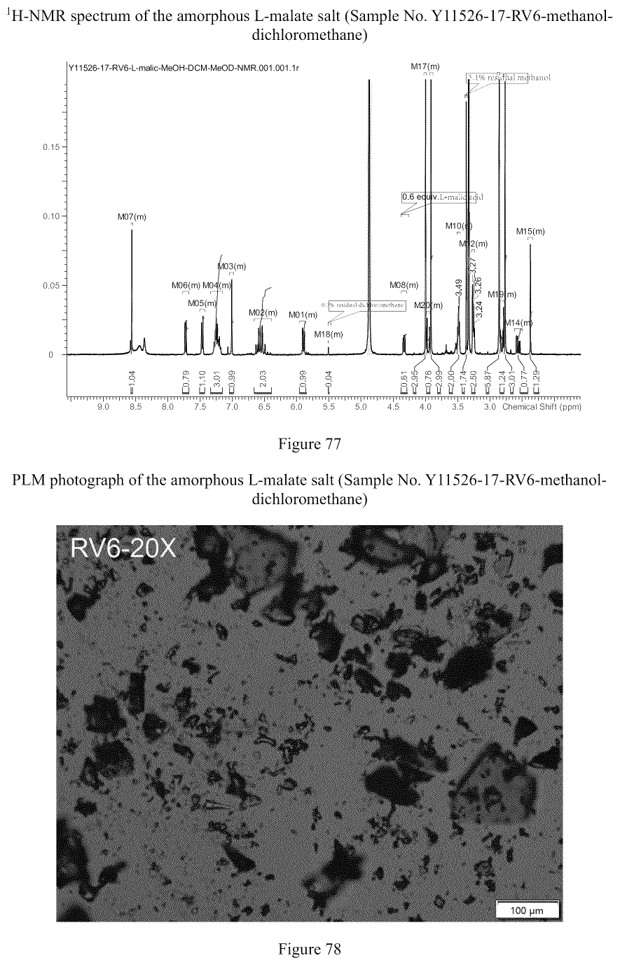

HPLC showed that the product had a purity of 91.1%, that is, the purity of the salt obtained at the end of the preparation was significantly reduced compared with the purity of the free base used to prepare the salt. PLM showed that the product was an irregular sample (FIG. 78). XRPD showed that the product was amorphous (FIG. 74). DSC showed that the product had a glass-transition temperature of 80.8° C. (FIG. 75). TGA showed that the product had a weight loss of about 3.3% at 120° C. (FIG. 76). ¹H-NMR showed a base:acid stoichiometric ratio of about 1:0.6 in the product (FIG. 77), which was unreasonable.

The amorphous L-malate salt (Sample No. Y11526-17-RV6-methanol-dichloromethane) was at least chemically unstable and unreasonable in terms of the base:acid stoichiometric ratio at the end of the preparation.

### Example 17: Amorphous 1 Eq. Citrate Salt (Sample No. Y11526-10-FD6-1,4-Dioxane)

About 100 mg of the compound of formula (I) (with a purity of 99.9%) were weighed and placed in a 40 mL glass bottle together with 1 eq. of citric acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous 1 eq. citrate salt.

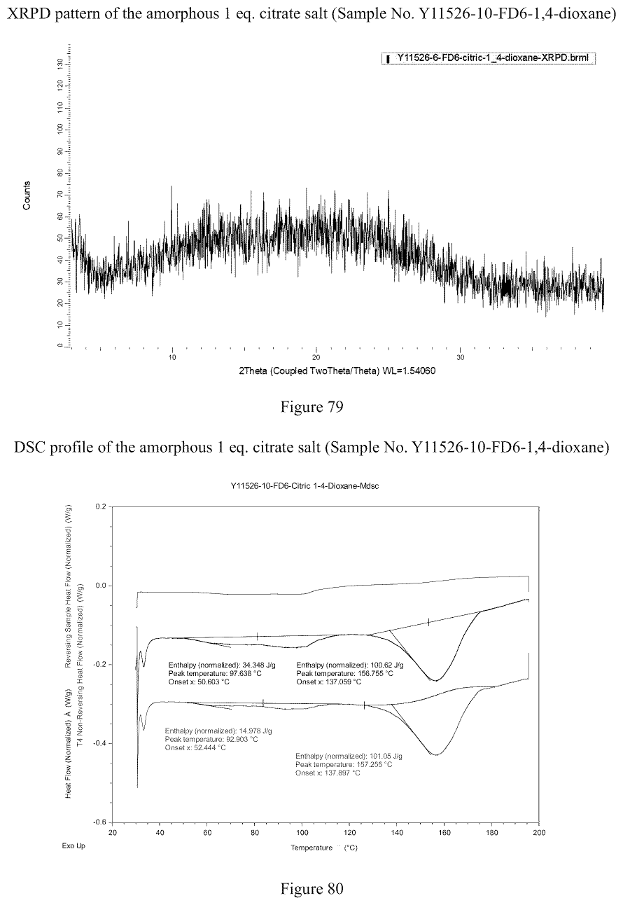

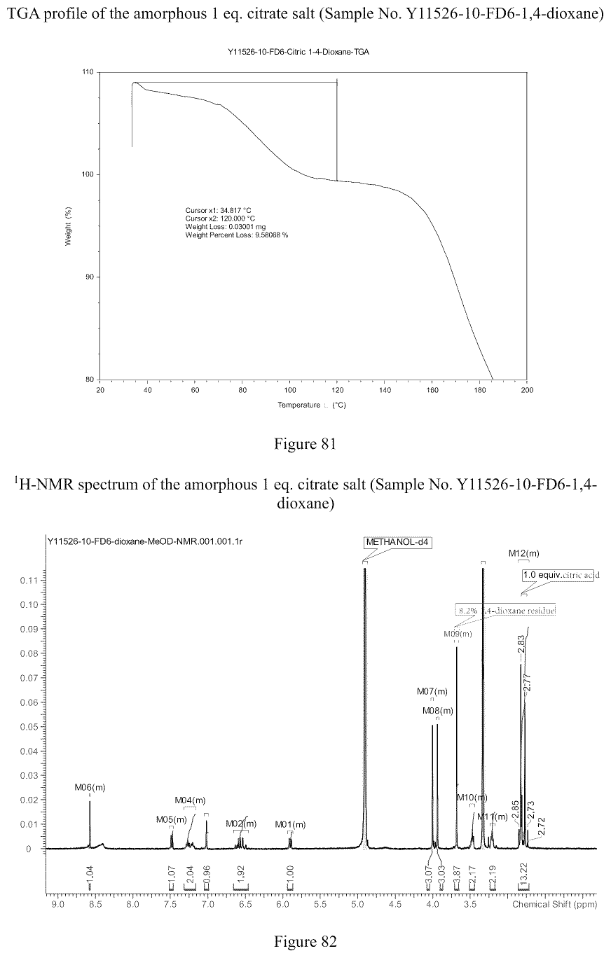

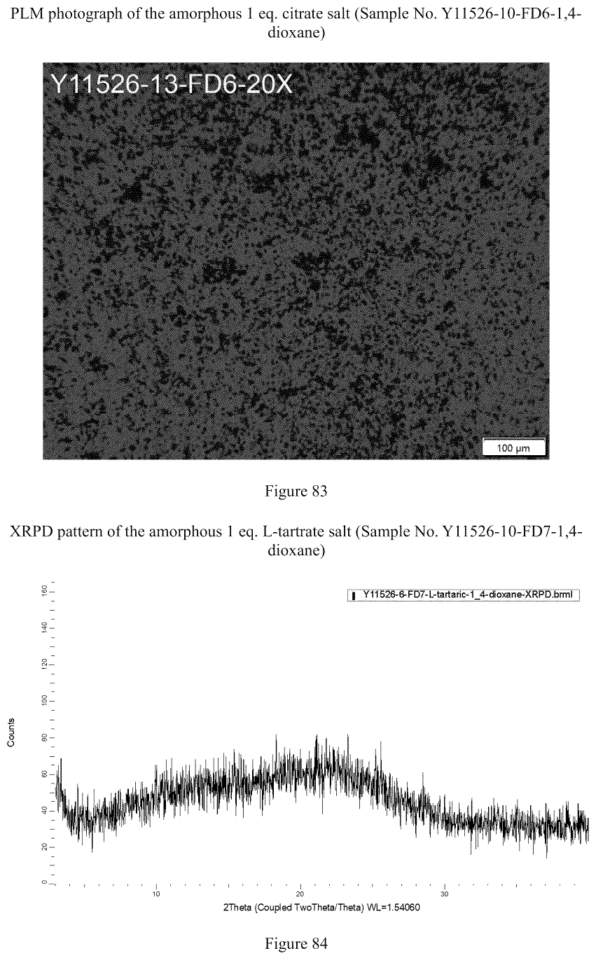

HPLC showed that the product had a purity of 94.4%, that is, the purity of the salt obtained at the end of the preparation was significantly reduced compared with the purity of the free base used to prepare the salt. PLM showed that the product was an irregular sample (FIG. 83). XRPD showed that the product was amorphous (FIG. 79). DSC showed that the product had no glass-transition temperature (FIG. 80). TGA showed that the product had a weight loss of about 9.6% at 120° C. (FIG. 81). ¹H-NMR showed a base:acid stoichiometric ratio of about 1:1 in the product (FIG. 82).

The amorphous 1 eq. citrate salt (Sample No. Y11526-10-FD6-1,4-dioxane) was reasonable in terms of the base:acid stoichiometric ratio, but at least chemically unstable at the end of the preparation.

### Example 18: Amorphous 1 Eq. L-Tartrate Salt (Sample No. Y11526-10-FD7-1,4-Dioxane)

About 100 mg of the compound of formula (I) (with a purity of 99.9%) were weighed and placed in a 40 mL glass bottle together with 1 eq. of L-tartaric acid, 10 mL of 1,4-dioxane were added, and the reaction was performed at 50° C. for 2 h. The resulting clear solution was pre-frozen in a dry ice/ethanol mixture for 2 h, and then 1,4-dioxane was removed by freeze drying to afford the amorphous 1 eq. L-tartrate.

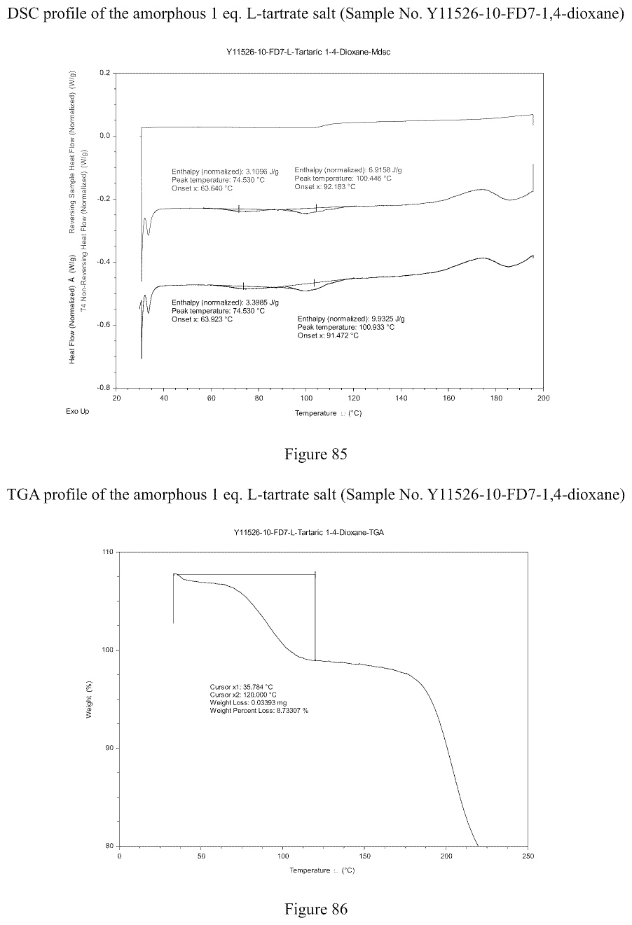

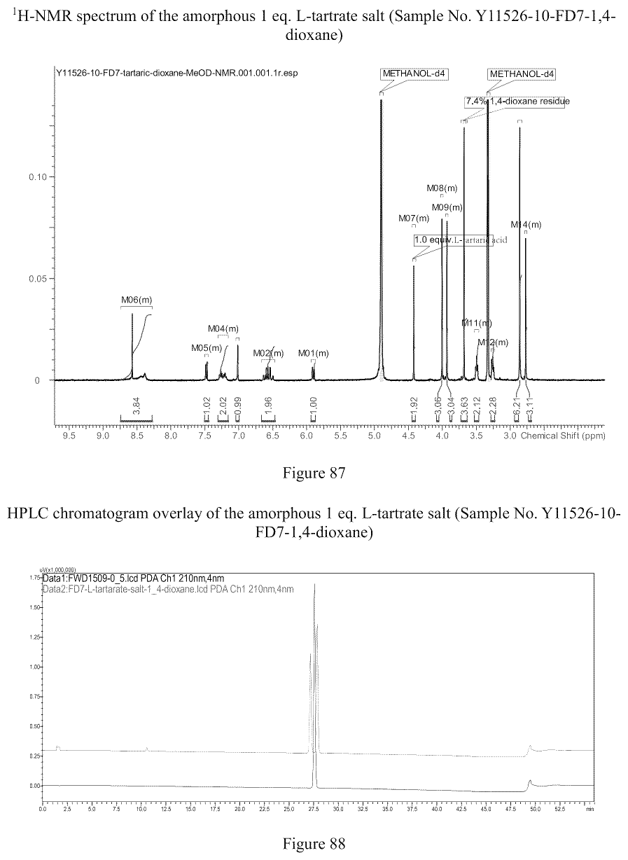

HPLC showed that the product was degraded (FIG. 88). XRPD showed that the product was amorphous (FIG. 84). DSC showed that the product had no glass-transition temperature (FIG. 85). TGA showed that the product had a weight loss of about 8.7% at 120° C. (FIG. 86). ¹H-NMR showed a base:acid stoichiometric ratio of about 1:1 in the product (FIG. 87).

The amorphous 1 eq. L-tartrate salt (Sample No. Y11526-10-FD7-1,4-dioxane) was reasonable in terms of the base:acid stoichiometric ratio, but at least chemically unstable at the end of the preparation.

It can be seen from Examples 1-18 that:
when the base:acid molar charge ratio was 1:1, sulfosalicylic acid and L-malic acid cannot enable the resulting salts to have a stoichiometric ratio of compound of formula (I):acid of 1:1;
when the base:acid molar charge ratio was 1:1, hydrochloric acid, methanesulfonic acid, benzenesulfonic acid, ethanesulfonic acid, maleic acid, hydrobromic acid, citric acid, and L-tartaric acid enable the resulting salts to have a stoichiometric ratio of compound of formula (I):acid of 1:1;
when the base:acid molar charge ratio was 1:2, nitric acid, sulfuric acid, and sulfosalicylic acid cannot enable the resulting salts to have a stoichiometric ratio of compound of formula (I):acid of 1:2; and
when the base:acid molar charge ratio was 1:2, hydrochloric acid, methanesulfonic acid, benzenesulfonic acid, and p-toluenesulfonic acid enable the resulting salts to have a stoichiometric ratio of compound of formula (I):acid of 1:2.

It can also be seen from Examples 1-18 that:
amorphous 1 eq. citrate salt, amorphous 1 eq. L-tartrate, amorphous nitrate salt and amorphous L-malate salt were chemically unstable at the end of the preparation;
amorphous 2 eq. p-tosylate salt and amorphous sulfate salt were physically unstable at the end of the preparation; and
amorphous 1 eq. hydrochloride salt, amorphous 2 eq. hydrochloride salt, amorphous 1 eq. mesylate salt, amorphous 2 eq. mesylate salt, amorphous 1 eq. besylate salt, amorphous 2 eq. besylate salt, amorphous 1 eq. esylate salt, amorphous 1 eq. maleate salt, crystalline 1 eq. hydrobromide salt, and amorphous sulfosalicylate salt were chemically and physically stable at the end of the preparation.

### Example 19: Solid Storage Stability Test

Each amorphous salt solid was weighed (two 2 mg samples were weighed for purity testing after solid storage stability study, and one 10 mg sample was weighed for XRPD testing after solid storage stability study) and placed in a glass vial. In a 25° C./60% RH solid storage stability test, the amorphous salt solid was placed in a 25° C./60% RH constant temperature and humidity chamber, and placed in the dark for 1 week (i.e. “solid/25° C./60% RH/open/1 week”), and in a 60° C. solid storage stability test, the amorphous salt solid was sealed in an oven at 60° C. and heated for 1 week in the dark (i.e. “solid/60° C./closed container/1 week”). Then the sample was taken out for purity testing, crystal form detection and appearance observation, respectively.

Examples 1-10 were subjected to the above solid storage stability tests, and the results are shown in Table 1.

TABLE 1
Results of the solid storage stability test

| | Examples | | | | |
|---|---|---|---|---|---|
| | 1 | 2 | 3 | 4 | 5 |
| | Sample | | | | |
| | amorphous free | amorphous 1 eq. | amorphous 2 eq | amorphous 1 eq. | amorphous 2 eq. |
| | base | hydrochloride salt | hydrochloride salt | mesylate salt | mesylate salt |
| Original purity | 99.9% | 99.9% | 99.9% | 99.9% | 99.9% |
| Original morphology | amorphous | amorphous | amorphous | amorphous | amorphous |

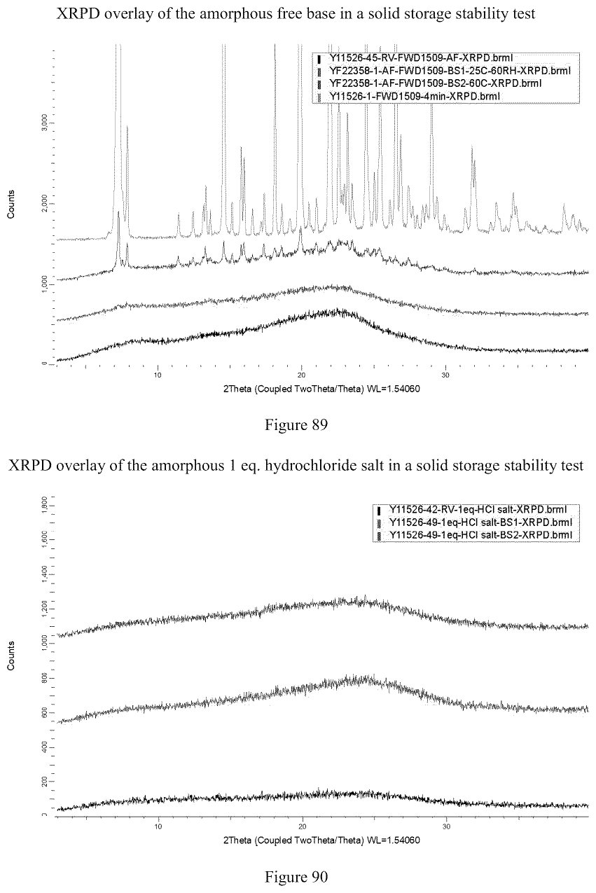

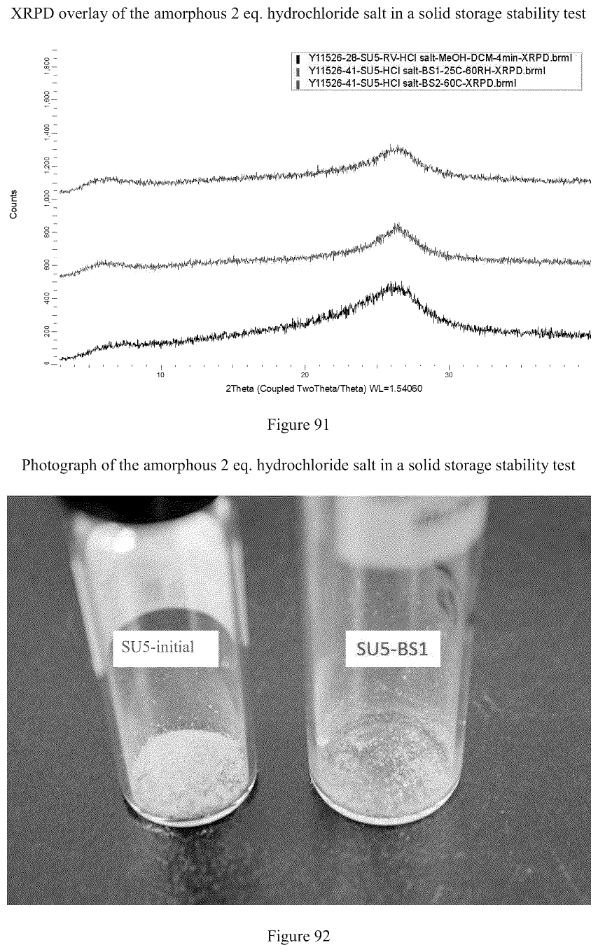

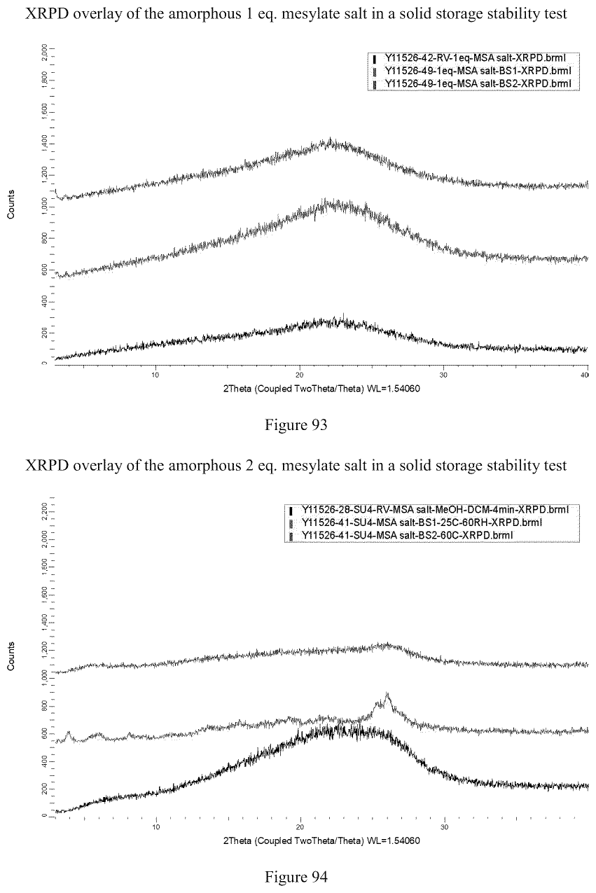

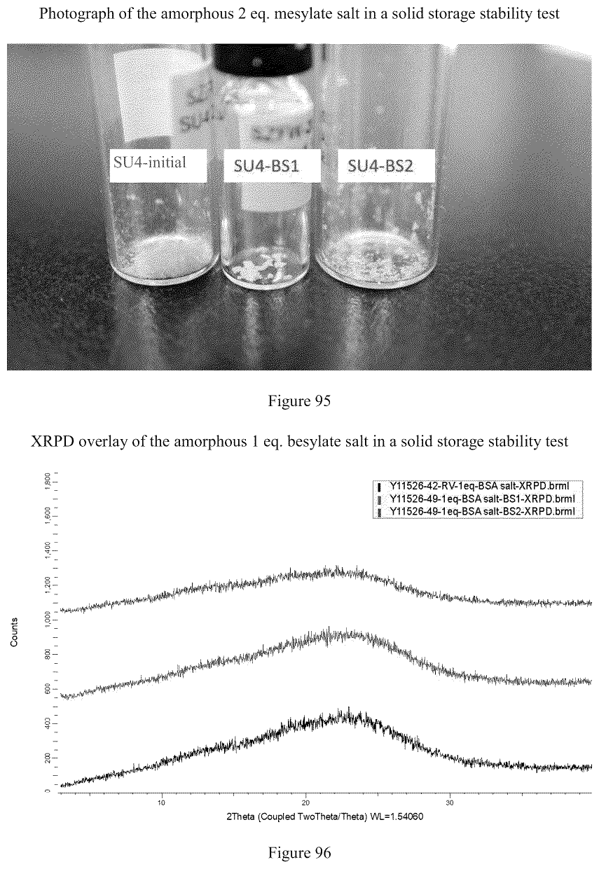

Test BS1: solid/25° C./60% RH/open/1 week

| | | | | | |
|---|---|---|---|---|---|
| Purity | 99.6% | 99.9% | 99.6% | 99.8% | 99.9% |
| Color | no color change | no color change | slight color change | no color change | no color change |
| Morphology | amorphous (FIG. 89), | amorphous (FIG. 90), | amorphous (FIG. 91), | amorphous (FIG. 93), | low crystallinity (FIG. |
| | no agglomeration | no agglomeration | partial agglomeration | no agglomeration | 94), agglomeration |
| | | | (FIG. 92) | | (FIG. 95) |

Test BS2: solid/60° C./closed container/1 week

| | | | | | |
|---|---|---|---|---|---|
| Purity | 99.9% | 98.2% | 99.6% | 99.9% | 99.8% |
| Color | no color change | no color change | no color change | no color change | no color change |
| Morphology | medium crystallinity | amorphous (FIG. 90), | amorphous (FIG. 91), | amorphous (FIG. 93), | amorphous (FIG. 94), |
| | (FIG. 89) | no agglomeration | no agglomeration | no agglomeration | partial agglomeration |
| | | | | | (FIG. 95) |

| | Examples | | | | |
|---|---|---|---|---|---|
| | 6 | 7 | 8 | 9 | 10 |
| | Sample | | | | |
| | amorphous 1 eq. | amorphous 2 eq. | amorphous 1 eq. | amorphous 1 eq. | crystalline 1 eq. |
| | besylate salt | besylate salt | esylate salt | maleate salt | hydrobromide salt |
| Original purity | 99.7% | 99.6% | 99.8% | 99.8% | 99.9% |
| Original morphology | amorphous | amorphous | amorphous | amorphous | crystalline |

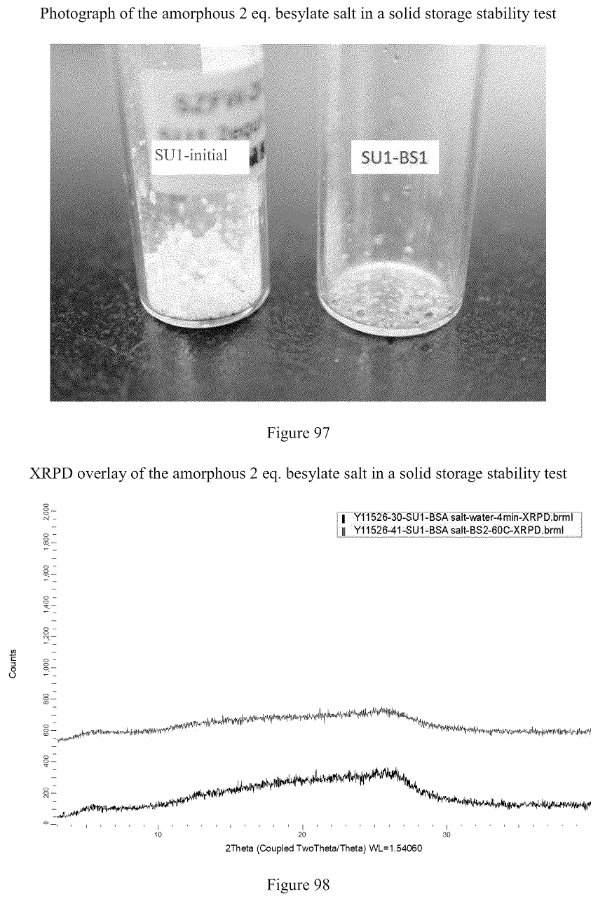

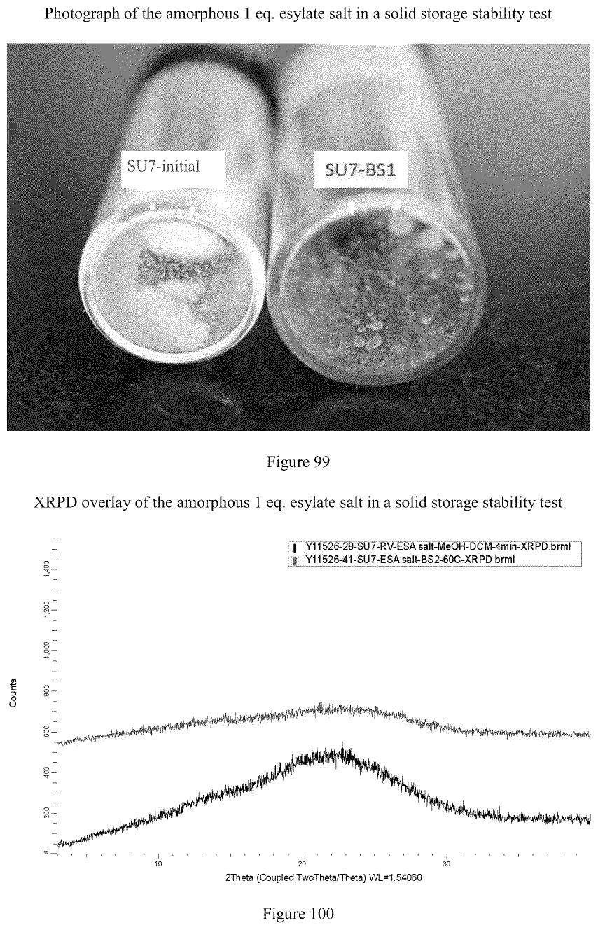

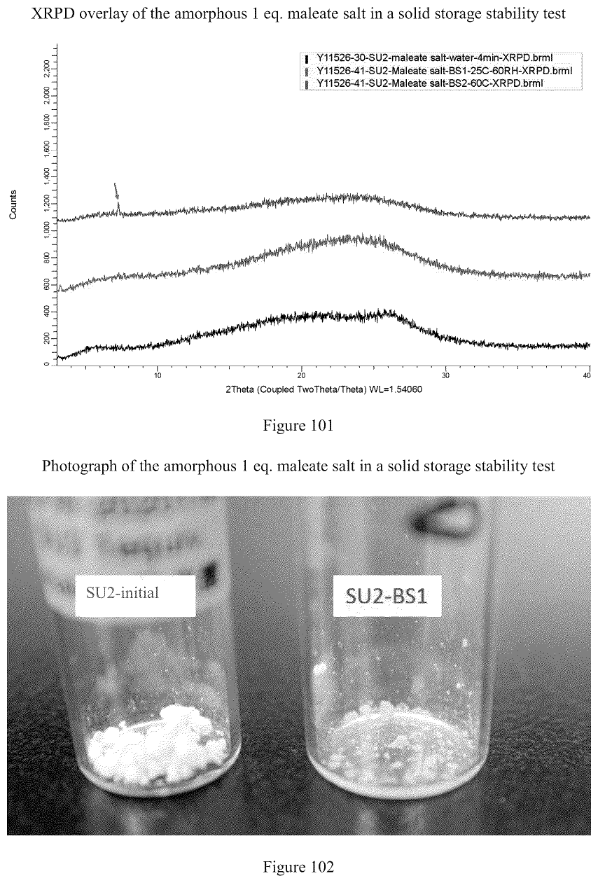

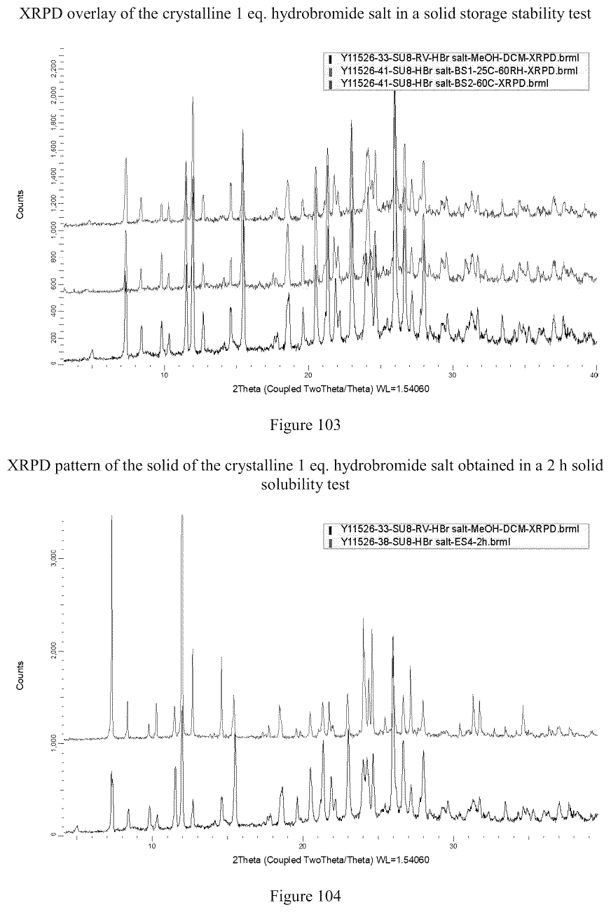

Test BS1: solid/25° C./60% RH/open/1 week

| | | | | | |
|---|---|---|---|---|---|
| Purity | 98.2% | 99.6% | 99.9% | 99.7% | 99.9% |
| Color | no color change | slight color change | no color change | slight color change | no color change |
| Morphology | amorphous (FIG. 96), | caking and fusion to a | caking and fusion to a | amorphous (FIG. 101), | no crystal form |
| | no agglomeration | glassy state (FIG. 97) | glassy state (FIG. 99) | agglomeration (FIG. 102) | change (FIG. 103) |

Test BS2: solid/60° C./closed container/1 week

| | | | | | |
|---|---|---|---|---|---|
| Purity | 99.6% | 99.4% | 99.8% | 98.2% | 99.9% |
| Color | no color change | no color change | no color change | no color change | no color change |
| Morphology | amorphous (FIG. 96), | amorphous (FIG. 98), | amorphous (FIG. 100), | low crystallinity | no crystal form |
| | no agglomeration | no agglomeration | no agglomeration | (FIG. 101) | change (FIG. 103) |

The amorphous free base showed good physical and chemical stability under the storage stability test conditions of “solid/25° C./60% RH/open/1 week”, and transformed from amorphous to a moderate crystallinity under the storage stability test conditions of “solid/60° C./closed container/1 week”.

The amorphous 1 eq. hydrochloride salt showed good physical and chemical stability under the storage stability test conditions of “solid/25° C./60% RH/open/1 week”, and had a decrease in purity (about 2%) under the storage stability test conditions of “solid/60° C./closed container/1 week”.

The amorphous 2 eq. hydrochloride salt had slight color change and showed a certain hygroscopicity (partial agglomeration) under the storage stability test conditions of “solid/25° C./60% RH/open/1 week”, and showed good physical and chemical stability under the storage stability test conditions of “solid/60° C./closed container/1 week”.

The amorphous 1 eq. mesylate salt showed good physical and chemical stability under the storage stability test conditions of both “solid/25° C./60% RH/open/1 week” and “solid/60° C./closed container/1 week”.

The amorphous 2 eq. mesylate salt transformed from amorphous to a low crystallinity and showed a certain hygroscopicity (agglomeration) under the storage stability test conditions of “solid/25° C./60% RH/open/1 week”, and showed a certain hygroscopicity (partial agglomeration) under the storage stability test conditions of “solid/60° C./closed container/1 week”.

The amorphous 1 eq. besylate salt had a decrease in purity (about 2%) under the storage stability test conditions of “solid/25° C./60% RH/open/1 week”, and showed good physical and chemical stability under the storage stability test conditions of “solid/60° C./closed container/1 week”.

The amorphous 2 eq. besylate salt had slight color change and showed significant hygroscopicity (caking and fusion to a glassy state) under the storage stability test conditions of “solid/25° C./60% RH/open/1 week”, and showed good physical and chemical stability under the storage stability test conditions of “solid/60° C./closed container/1 week”.

The amorphous 1 eq. esylate salt showed significant hygroscopicity (caking and fusion to a glassy state) under the storage stability test conditions of “solid/25° C./60% RH/open/1 week”, and showed good physical and chemical stability under the storage stability test conditions of “solid/60° C./closed container/1 week”.

The amorphous 1 eq. maleate salt had slight color change and showed a certain hygroscopicity (agglomeration) under the storage stability test conditions of “solid/25° C./60% RH/open/1 week”, and had a decrease in purity (about 2%) and transformed from amorphous to a low crystallinity under the storage stability test conditions of “solid/60° C./closed container/1 week”.

The crystalline 1 eq. hydrobromide salt showed good physical and chemical stability under the storage stability test conditions of both “solid/25° C./60% RH/open/1 week” and “solid/60° C./closed container/1 week”.

It can be seen from Example 19 that:
The amorphous 1 eq. besylate salt, amorphous 1 eq. hydrochloride salt and amorphous 1 eq. maleate salt were chemically unstable under the storage conditions of “solid/25° C./60% RH/open/1 week” and/or “solid/60° C./closed container/1 week”;
the amorphous 2 eq. hydrochloride salt, amorphous 2 eq. mesylate salt, amorphous 2 eq. besylate salt, amorphous 1 eq. esylate salt, and amorphous 1 eq. maleate salt were physically unstable under the storage conditions of “solid/25° C./60% RH/open/1 week” and/or “solid/60° C./closed container/1 week”; and
the amorphous 1 eq. mesylate salt and crystalline 1 eq. hydrobromide salt were chemically and physically stable under the storage conditions of “solid/25° C./60% RH/open/1 week” and “solid/60° C./closed container/1 week”.

### Example 20: Solid Solubility Test

5 samples were weighed for each amorphous salt solid (equivalent to 20 mg of free base), and 10 mL of the following solvents were respectively added: 0.1N HCl solution (pH 1.0), 50 mM phosphate buffer (pH 4.5), FeSSIF-V1 (pH 5.0), FaSSIF-V1 (pH 6.5) and SGF (pH 2.0), and stirred at 37° C. for 2 h. The suspension was then centrifuged at 37° C., the supernatant was measured for solubility by HPLC, and the solid fraction was measured for XRPD. The target solubility was at least 2 mg (based on free base)/mL.

Examples 1-10 were subjected to the above 2 h solid solubility test, and the results are shown in Table 2. Similarly, Examples 1 and 4 were subjected to a 24 h solid solubility test, and the results are shown in Table 3.

TABLE 2
Results of the 2 h solid solubility test
Examples

| | | | |
|---|---|---|---|
| 1 | 2 | 3 | 4 |

Sample

| | | amorphous 1 eq. | | amorphous 2 eq. | | amorphous 1 eq. | |
|---|---|---|---|---|---|---|---|
| amorphous free base | | hydrochloride salt | | hydrochloride salt | | mesylate salt | |
| Solubility | XRPD | Solubility | XRPD | Solubility | XRPD | Solubility | XRPD |
| measured at | measured at | measured at | measured at | measured at | measured at | measured at | measured at |
| 2 h (pH) | 2 h | 2 h (pH) | 2 h | 2 h (pH) | 2 h | 2 h (pH) | 2 h |

Test ES1: 0.1N HCl solution (pH 1.0)

| | | | | | | | |
|---|---|---|---|---|---|---|---|
| >2 mg/mL | clear solution | >2 mg/mL | clear solution | >2 mg/mL | clear solution | >2 mg/mL | clear solution |
| (0.8) | not performed | (0.7) | not performed | (0.7) | not performed | (0.7) | not performed |

Test ES2: 50 mM phosphate buffer (pH 4.5)

| | | | | | | | |
|---|---|---|---|---|---|---|---|
| >2 mg/mL | clear solution | >2 mg/mL | clear solution | >2 mg/mL | clear solution | >2 mg/mL | clear solution |
| (4.6) | not performed | (4.5) | not performed | (4.2) | not performed | (4.5) | not performed |

Test ES3: FeSSIF-V1 (pH 5.0)

| | | | | | | | |
|---|---|---|---|---|---|---|---|
| >2 mg/mL | clear solution | >2 mg/mL | clear solution | >2 mg/mL | clear solution | >2 mg/mL | clear solution |
| (4.9) | not performed | (4.8) | not performed | (4.8) | not performed | (4.8) | not performed |

Test ES4: FaSSIF-V1 (pH 6.5)

| | | | | | | | |
|---|---|---|---|---|---|---|---|
| >2 mg/mL | clear solution | >2 mg/mL | clear solution | >2 mg/mL | clear solution | >2 mg/mL | clear solution |
| (6.8) | not performed | (6.5) | not performed | (6.1) | not performed | (6.5) | not performed |

Test ES5: SGF (pH 2.0)

| | | | | | | | |
|---|---|---|---|---|---|---|---|
| >2 mg/mL | clear solution | >2 mg/mL | clear solution | >2 mg/mL | clear solution | >2 mg/mL | clear solution |
| (2.4) | not performed | (2.0) | not performed | (1.8) | not performed | (2.0) | not performed |

Examples

| | | |
|---|---|---|
| 5 | 6 | 7 |

Sample

| amorphous 2 eq. | | amorphous 1 eq. | | amorphous 2 eq. | |
|---|---|---|---|---|---|
| mesylate salt | | besylate salt | | besylate salt | |
| Solubility | XRPD | Solubility | XRPD | Solubility | XRPD |
| measured at | measured at | measured at | measured at | measured at | measured at |
| 2 h (pH) | 2 h | 2 h (pH) | 2 h | 2 h (pH) | 2 h |
| Test ES1: 0.1N HCl solution (pH 1.0) | | | | | |
| >2 mg/mL | clear solution | >2 mg/mL | clear solution | 1.8 mg/mL | nearly clear solution |
| (0.7) | not performed | (0.7) | not performed | (0.8) | a small amount of |
| | | | | | floc not performed |
| Test ES2: 50 mM phosphate buffer (pH 4.5) | | | | | |
| >2 mg/mL | clear solution | >2 mg/mL | clear solution | >2 mg/mL | clear solution |
| (4.2) | not performed | (4.5) | not performed | (4.2) | not performed |
| Test ES3: FeSSIF-V1 (pH 5.0) | | | | | |
| >2 mg/mL | clear solution | >2 mg/mL | clear solution | >2 mg/mL | clear solution |
| (4.8) | not performed | (4.8) | not performed | L(4.8) | not performed |
| Test ES4: FaSSIF-V1 (pH 6.5) | | | | | |
| >2 mg/mL | clear solution | >2 mg/mL | clear solution | >2 mg/mL | clear solution |
| (6.2) | not performed | (6.5) | not performed | (6.1) | not performed |
| Test ES5: SGF (pH 2.0) | | | | | |
| >2 mg/mL | clear solution | >2 mg/mL | clear solution | >2 mg/mL | clear solution |
| (1.8) | not performed | (2.1) | not performed | (1.7) | not performed |

Examples

| | | |
|---|---|---|
| 8 | 9 | 10 |

Sample

| amorphous 1 eq. | | amorphous 1 eq. | | crystalline 1 eq. | |
|---|---|---|---|---|---|
| esylate salt | | maleate salt | | hydrobromide salt | |
| Solubility | XRPD | Solubility | XRPD | Solubility | XRPD |
| measured at | measured at | measured at | measured at | measured at | measured at |
| 2 h (pH) | 2 h | 2 h (pH) | 2 h | 2 h (pH) | 2 h |

Test ES1: 0.1N HCl solution (pH 1.0)

| | | | | | |
|---|---|---|---|---|---|
| >2 mg/mL | clear solution | >2 mg/mL | clear solution | >2 mg/mL | clear solution |
| (0.8) | not performed | (0.8) | not performed | (0.8) | not performed |

Test ES2: 50 mM phosphate buffer (pH 4.5)

| | | | | | |
|---|---|---|---|---|---|
| >2 mg/mL | clear solution | >2 mg/mL | clear solution | >2 mg/mL | clear solution |
| (4.4) | not performed | (4.3) | not performed | (4.4) | not performed |

Test ES3: FeSSIF-V1 (pH 5.0)

| | | | | | |
|---|---|---|---|---|---|
| >2 mg/mL | clear solution | >2 mg/mL | clear solution | >2 mg/mL | clear solution |
| (4.8) | not performed | (4.8) | not performed | (4.8) | not performed |

Test ES4: FaSSIF-V1 (pH 6.5)

| | | | | | |
|---|---|---|---|---|---|
| >2 mg/mL | clear solution | >2 mg/mL | clear solution | 0.7 mg/mL | hydrobromide |
| (6.4) | not performed | (6.1) | not performed | (6.4) | salt FIG. 104 |

Test ES5: SGF (pH 2.0)

| | | | | | |
|---|---|---|---|---|---|
| >2 mg/mL | clear solution | >2 mg/mL | clear solution | >2 mg/ml | clear solution |
| (1.9) | not performed | (2.0) | not performed | (1.9) | not performed |

TABLE 3
Results of the 24 h solid solubility test

| Examples | |
|---|---|
| 1 | 4 |
| Sample | |
| amorphous free base | amorphous 1 eq. mesylate salt |

| | | | |
|---|---|---|---|
| Solubility | XRPD | Solubility | XRPD |
| measured at | measured at | measured at | measured at |
| 24 h (pH) | 24 h | 24 h (pH) | 24 h |

Test ES3: FeSSIF-VI (pH 5.0)

| | | | |
|---|---|---|---|
| >2 mg/mL | clear solution | >2 mg/mL | clear solution |
| (5.0) | not performed | (5.0) | not performed |

Test ES4: FaSSIF-VI (pH 6.5)

| | | | |
|---|---|---|---|
| >2 mg/mL | clear solution | >2 mg/mL | clear solution |
| (6.8) | not performed | (6.6) | not performed |

In the 2 h solid solubility test, the amorphous 2 eq. besylate salt did not achieve a solubility of 2 mg/mL in the 0.1N HCl solution (pH 1.0), the crystalline 1 eq. hydrobromide salt did not achieve a solubility of 2 mg/mL in FaSSIF-V1 (pH 6.5), and the amorphous 1 eq. hydrochloride salt, amorphous 2 eq. hydrochloride salt, amorphous 1 eq. mesylate salt, amorphous 2 eq. mesylate salt, amorphous 1 eq. besylate salt, amorphous 1 eq. esylate salt, and amorphous 1 eq. maleate salt had a solubility greater than 2 mg/mL in all solvents tested. Additionally, the amorphous free base and the amorphous 1 eq. mesylate salt had a solubility greater than 2 mg/mL in FeSSIF-V1 (pH 5.0) and FaSSIF-V1 (pH 6.5) in the 24 h solid solubility test.

It can be seen from Examples 1-20 that the amorphous 1 eq. mesylate salt had a reasonable salt-forming equivalent ratio, better chemical stability, better physical stability and better solubility simultaneously.

### Example 21: Synthesis of N-(2-((2-(dimethylamino)ethyl(methyl)amino)-4-methoxy-5-((4-(1-methyl-1H-indole-3-yl)-1,3,5-triazin-2-yl)amino)phenyl)acrylamide

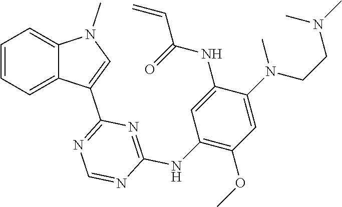 [SMILES: C=CC(=O)Nc1cc(Nc2ncnc(-c3cn(C)c4ccccc34)n2)c(OC)cc1N(C)CCN(C)C]

### A. Synthesis of Key Intermediate 6

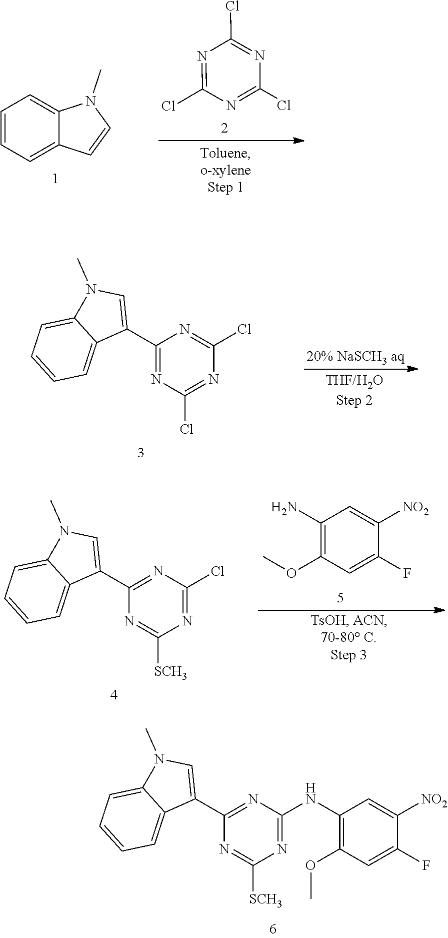

Step 1: Toluene (6V) and o-xylene (6V) were added to a reaction flask, stirred, and heated up to 60-70° C., and Starting material 1 (1.0 eq.) and Compound 2 (1.2 eq.) were added successively to the reaction flask, heated up to 110-120° C., reacted under stirring for 16-19 h, cooled down to 20-30° C. and stirred for crystallization for 3 h, and filtered. The filter cake was rinsed twice with acetonitrile (1V*2), and dried to constant weight to obtain Intermediate 3 with a purity of 96% in a yield of 75%.

¹H NMR (CDCl₃, 400 MHZ): δ 3.91 (s, 3H), 7.35-7.44 (m, 3H), 8.30 (s, 1H), 8.50-8.55 (m, 1H) ppm.

Step 2: The Intermediate 3 (1.0 eq.) and tetrahydrofuran (20V) were added to the reaction flask and stirred at 0-20° C. 10% NaSCH₃ (1.1 eq.) was slowly added dropwise to the reaction flask with the temperature controlled at 0-20° C., and reacted under stirring for 1-2 h, and water (40V) was added to the reaction mixture, stirred with the temperature controlled at 0-20° C., and filtered. The filter cake was rinsed twice with water (1V*2), and dried to constant weight to obtain the crude product. The crude product and DMA (8V) were added to the reaction flask successively and heated up to 70-80° C. for dissolved clarification. ACN (12V) was slowly added to the reaction flask with the temperature controlled at 70-80° C., stirred for 30 min, cooled down to 20-30° C., stirred for 1-2 h, and filtered. The filter cake was washed with acetonitrile (1V*2), and dried to constant weight to obtain Intermediate 4 with a purity of 98% in a yield of 68%.

¹H NMR (DMSO-d₆, 400 MHZ): δ 2.66 (s, 3H), 3.93 (s, 3H), 7.31-7.34 (m, 2H), 7.60-7.62 (m, 1H), 8.34-8.38 (m, 2H), 8.60 (s, 1H) ppm.

Step 3: ACN (15V) and Starting material 5 (1.1 eq.) were added to the reaction flask, and then TsOH (0.2 eq.) and Intermediate 4 (1.0 eq.) were added to the reaction flask, heated up to 70-80° C., reacted under stirring for 4-6 h, and filtered while hot. ACN (5V) was added to the filter cake, heated up to 60-70° C. and stirred for 30 min, and filtered while hot, and the filter cake was washed twice with ACN (2V*2). The filter cake was dried to constant weight to obtain Intermediate 6 with a purity of 97% in a yield of 86%.

¹H NMR (DMSO-d₆, 400 MHZ): δ 2.58 (s, 1H), 3.90 (s, 1H), 3.99 (s, 1H), 7.17 (s, 1H), 7.27 (t, J=7.50 Hz, 1H), 7.40 (d, J=13.38 Hz, 1H), 7.54 (d, J=8.25 Hz, 1H), 8.36 (s, 1H), 8.86 (d, J=8.50 Hz, 1H), 9.16 (s, 1H) ppm.

### B. Synthesis of the Target Compound N-(2-((2-(dimethylamino)ethyl(methyl)amino)-4-methoxy-5-((4-(1-methyl-1H-indole-3-yl)-1,3,5-triazin-2-yl)amino)phenyl)acrylamide

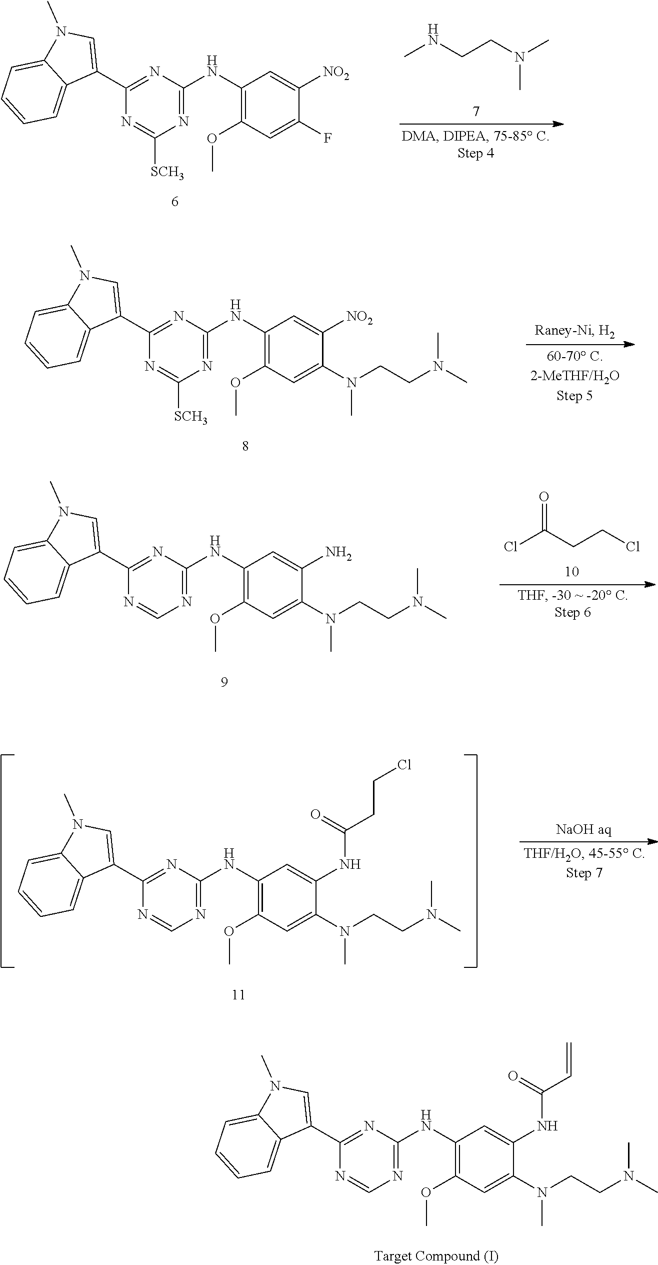

Step 4: DMA (4.68 g) was weighed into the reaction flask, stirred and heated up to 50-60° C. DIPEA (0.44 g) and Starting material 7 (0.30 g) were added to the reaction and stirred at 50-60° C. Intermediate 6 (1 g) was weighed, added to the reaction system at 50-60° C. and stirred. The reaction system was heated up to 75-85° C., the reaction mixture was stirred at 75-85° C. for 4-5 h, and a sample was taken and sent to HPLC for in-process control. If the Intermediate 6 was ≤0.5%, the reaction was stopped (if the Intermediate 6 was >0.5%, the reaction time was prolonged, and the sample was taken every 1-2 h, until the Intermediate 6 was ≤0.5%, and the reaction was stopped). The reaction mixture was subjected to silica gel column chromatography to obtain Intermediate 8 with a purity of 99% in a yield of 72%.

¹H NMR (DMSO-d₆, 400 MHZ): δ 2.16 (s, 6H), 2.46-2.49 (m, 2H), 2.55 (s, 3H), 2.88 (s, 3H), 3.30 (t, 2H), 3.88 (s, 3H), 3.92 (s, 3H), 6.82 (s, 1H), 7.24 (m, 2H), 7.52 (d, J=8.00 Hz, 1H), 8.32-8.40 (m, 3H), 8.90 (s, 1H) ppm.

Step 5: Raney-Ni (4.00 g) and water (4.50 g) were weighed into an autoclave, Intermediate 8 (1 g) was weighed, added to 2-MeTHF (12.90 g) and stirred for use. The mixture of Intermediate 8 and 2-MeTHF was poured into a 10 L autoclave, and the reaction system in the autoclave was purged with argon or nitrogen 3 times, then purged with hydrogen 3 times, with the hydrogen pressure controlled at 0.2-0.6 Mpa, and stirred. The temperature was increased to 60-70° C., the hydrogen pressure was 0.2-0.6 Mpa, and the reaction was stirred for 10-12 h. The reaction mixture was subjected to silica gel column chromatography to obtain Intermediate 9 with a purity of 98% in a yield of 63%.

¹H NMR (DMSO-d₆, 400 MHZ): δ 2.18 (s, 6H), 2.38 (t, J=6.57 Hz, 2H), 2.65 (s, 3H), 2.91 (t, J=6.38 Hz, 2H), 3.35 (s, 3H), 3.88 (s, 3H), 4.67 (br s, 2H), 6.77 (s, 1H), 7.12 (s, 2H), 7.24 (t, J=7.44 Hz, 1H), 7.50 (d, J=8.25 Hz, 1H), 8.32 (br s, 2H), 8.53 (s, 1H), 8.67 (s, 1H) ppm.

Step 6: Tetrahydrofuran (8.90 g) was weighed, added to a glass reaction flask and stirred, and Intermediate 9 (1 g) was weighed and added to the reaction flask and stirred until dissolved. The mixture was cooled down to −30° C. to −20° C., and 3-chloropropionyl chloride (0.34 g) was weighed, slowly added dropwise to the reaction flask at −30° C. to −20° C., and stirred for reaction at −30° C. to −10° C. for 0.5-1.0 h.

Step 7: A sodium hydroxide solution (sodium hydroxide (0.45 g) was dissolved in water (2.00 g) and stirred until dissolved completely) was added to the reaction flask and heated up to 45-55° C., at which temperature the reaction was kept under stirring for 12-14 h. The reaction mixture was extracted and subjected to silica gel column chromatography to obtain the target compound of formula (I) with a purity of 99% in a yield of 72%.

¹H NMR (CDCl₃, 400 MHZ): δ 10.15 (s, 1H), 9.92 (s, 1H), 9.38 (s, 1H), 8.74 (s, 1H), 8.65 (dd, J=6.1, 2.9 Hz, 1H), 7.80 (s, 1H), 7.41 (dd, J=6.3, 2.8 Hz, 1H), 7.37-7.22 (m, 2H), 6.83 (s, 1H), 6.57-6.46 (m, 1H), 6.41 (dd, J=16.8, 9.8 Hz, 1H), 5.77 (dd, J=9.8, 1.9 Hz, 1H), 4.02 (s, 3H), 3.93 (s, 3H), 2.37 (s, 2H), 2.36 (d, J=8.7 Hz, 6H) ppm.

### Example 22: Preparation of the Crystalline Form II-A of the Mesylate Salt of N-(2-((2-(dimethylamino)ethyl(methyl)amino)-4-methoxy-5-((4-(1-methyl-1H-indole-3-yl)-1,3,5-triazin-2-yl)amino)phenyl)acrylamide

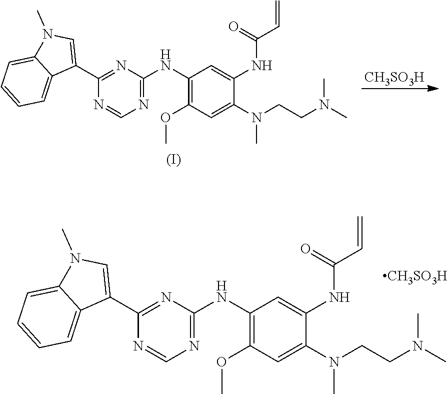

800 mg of N-(2-((2-(dimethylamino)ethyl(methyl)amino)-4-methoxy-5-((4-(1-methyl-1H-indole-3-yl)-1,3,5-triazin-2-yl)amino)phenyl)acrylamide were added to a round-bottom flask, 0.4 mL of water was added to the round-bottom flask, 152 mg of methanesulfonic acid dissolved in 5 mL of acetone were added to the round-bottom flask, and after suspending and stirring at room temperature for about 3 days, the solid was separated by centrifugation and dried under vacuum at 50° C. for 1 h to obtain the target product. The yield was 85.0%.

¹H NMR (CDCl₃, 400 MHZ): δ 10.15 (s, 1H), 9.92 (s, 1H), 9.38 (s, 1H), 8.74 (s, 1H), 8.65 (dd, J=6.1, 2.9 Hz, 1H), 7.80 (s, 1H), 7.41 (dd, J=6.3, 2.8 Hz, 1H), 7.37-7.22 (m, 2H), 6.83 (s, 1H), 6.57-6.46 (m, 1H), 6.41 (dd, J=16.8, 9.8 Hz, 1H), 5.77 (dd, J=9.8, 1.9 Hz, 1H), 4.02 (s, 3H), 3.93 (s, 3H), 2.30 (s, 3H), 2.37 (s, 2H), 2.36 (d, J=8.7 Hz, 6H) ppm.

After testing, the powder X-ray diffraction of the crystalline form II-A obtained in this example had characteristic peaks at diffraction angle 2θ° of 7.22°±0.2°, 8.62°±0.2°, 9.48°±0.2°, 10.36°±0.2°, 15.12°±0.2°, 15.74°±0.2°, 16.46°±0.2°, 16.92°±0.2°, 17.70°±0.2°, 18.94°±0.2°, 19.30°±0.2°, 19.62°±0.2°, 20.36°±0.2°, 20.81°±0.2°, 22.28°±0.2°, 24.06°±0.2°, 24.78°±0.2°, 25.54°±0.2°, 25.82°±0.2°, and 26.24°±0.2°. Its X-ray powder diffraction pattern (XRPD) is shown in FIG. 105.

After testing, the crystalline form II-A was obtained in this example, and had a weight loss of about 0.56% when heated to 200.0° C.; and the crystalline form II-A began to show an endothermic peak when heated to 255.9° C., with its thermogravimetric analysis (TGA)/differential scanning calorimetry (DSC) profile substantially as shown in FIG. 106.

### Example 23: Preparation of the Crystalline Form III-A of the Hydrochloride Salt of N-(2-((2-(dimethylamino)ethyl(methyl)amino)-4-methoxy-5-((4-(1-methyl-1H-indole-3-yl)-1,3,5-triazin-2-yl)amino)phenyl)acrylamide

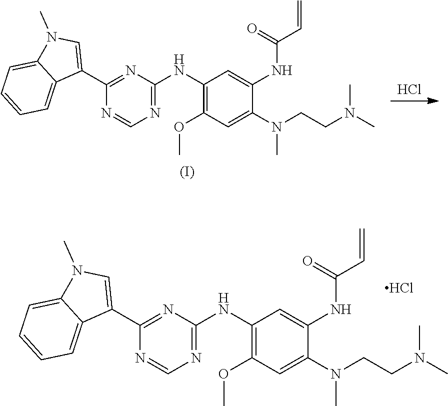

800 mg of N-(2-((2-(dimethylamino)ethyl(methyl)amino)-4-methoxy-5-((4-(1-methyl-1H-indole-3-yl)-1,3,5-triazin-2-yl)amino)phenyl)acrylamide were added to a round-bottom flask, 132 μL of concentrated hydrochloric acid (about 37%) dissolved in 5 mL of ethanol were added to the round-bottom flask, and after suspending and stirring at room temperature for about 3 days, the solid was separated by centrifugation and dried under vacuum at 50° C. for 1 h to obtain the target product. The yield was 79.4%.

¹H NMR (CDCl₃, 400 MHZ): δ 10.15 (s, 1H), 9.92 (s, 1H), 9.38 (s, 1H), 8.74 (s, 1H), 8.65 (dd, J=6.1, 2.9 Hz, 1H), 7.80 (s, 1H), 7.41 (dd, J=6.3, 2.8 Hz, 1H), 7.37-7.22 (m, 2H), 6.83 (s, 1H), 6.57-6.46 (m, 1H), 6.41 (dd, J=16.8, 9.8 Hz, 1H), 5.77 (dd, J=9.8, 1.9 Hz, 1H), 4.02 (s, 3H), 3.93 (s, 3H), 2.37 (s, 2H), 2.36 (d, J=8.7 Hz, 6H) ppm.

After testing, the powder X-ray diffraction of the crystalline form III-A obtained in this example had characteristic peaks at 2θ° of 6.55°±0.2°, 10.36°±0.2°, 12.28°±0.2°, 13.10°±0.2°, 14.08°±0.2°, 14.45°±0.2°, 15.98°±0.2°, 17.33°±0.2°, 19.07°±0.2°, 19.57°±0.2°, 21.15°±0.2°, 21.72°±0.2°, 24.30°±0.2°, 24.85°±0.2°, 25.87°±0.2°, and 33.29°±0.2°. Its X-ray powder diffraction pattern (XRPD) is shown in FIG. 107.

After testing, the crystalline form III-A was obtained in this example, and had a weight loss of about 1.05% when heated to 150.0° C.; and the crystalline form III-A began to show an endothermic peak when heated to 193.3° C., with its thermogravimetric analysis (TGA)/differential scanning calorimetry (DSC) profile substantially as shown in FIG. 108.

### Example 24: Preparation of the Crystalline Form IV-C of the Maleate Salt of N-(2-((2-(dimethylamino)ethyl(methyl)amino)-4-methoxy-5-((4-(1-methyl-1H-indole-3-yl)-1,3,5-triazin-2-yl)amino)phenyl)acrylamide

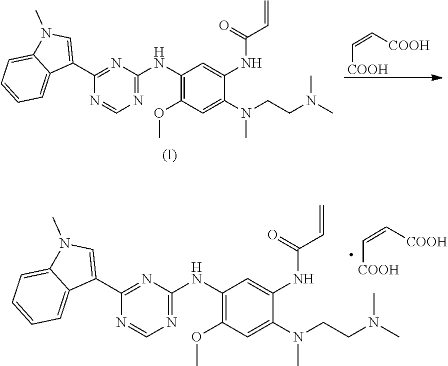

800 mg of N-(2-((2-(dimethylamino)ethyl(methyl)amino)-4-methoxy-5-((4-(1-methyl-1H-indole-3-yl)-1,3,5-triazin-2-yl)amino)phenyl)acrylamide were added to a round-bottom flask, 0.4 mL of water was added to the round-bottom flask, 184 mg of maleic acid dissolved in 5 mL of ethanol were added to the round-bottom flask, and after suspending and stirring at room temperature for about 3 days, the solid was separated by centrifugation and dried under vacuum at 50° C. for 1 h to obtain the target product. The yield was 83.8%.

¹H NMR (CDCl₃, 400 MHZ): δ 10.15 (s, 1H), 9.92 (s, 1H), 9.38 (s, 1H), 8.74 (s, 1H), 8.65 (dd, J=6.1, 2.9 Hz, 1H), 7.80 (s, 1H), 7.41 (dd, J=6.3, 2.8 Hz, 1H), 7.37-7.22 (m, 2H), 6.83 (s, 1H), 6.57-6.46 (m, 1H), 6.41 (dd, J=16.8, 9.8 Hz, 1H), 6.02 (s, 2H), 5.77 (dd, J=9.8, 1.9 Hz, 1H), 4.02 (s, 3H), 3.93 (s, 3H), 2.37 (s, 2H), 2.36 (d, J=8.7 Hz, 6H) ppm.

After testing, the powder X-ray diffraction of the crystalline form IV-C obtained in this example had characteristic peaks at diffraction angle 2θ° of 6.47°±0.2°, 8.44°±0.2°, 9.15°±0.2°, 10.63°±0.2°, 12.70°±0.2°, 15.48°±0.2°, 16.42°±0.2°, 17.00°±0.2°, 18.19°±0.2°, 20.71°±0.2°, 22.43°±0.2°, 25.76°±0.2°, 28.70°±0.2°, and 31.39°±0.2°. Its X-ray powder diffraction pattern (XRPD) is shown in FIG. 109.

After testing, the crystalline form IV-C was obtained in this example, and had a weight loss of about 1.96% when heated to 220.0° C.; and the crystalline form IV-C began to show an endothermic peak when heated to 255.5° C., with its thermogravimetric analysis (TGA)/differential scanning calorimetry (DSC) profile substantially as shown in FIG. 110.

### Test Example 1: Drug Absorption Test in Male Han-Wister Rats

Intravenous administration: Three healthy male Han-Wister rats having a body weight of 250-350 g were provided by Beijing Vital River Laboratory Animal Technology Co., Ltd. The above compound of Example 1 was administered intravenously at the doses (calculated based on free base) listed in Table 1 below, respectively. Before administration and 15 min, 0.5 h, 1.0 h, 2.0 h, 4.0 h, 8.0 h, 12 h, and 24 h after administration, jugular vein puncture was performed to take 0.05 mL of blood, plasma was separated and prepared, and the drug concentration in the plasma was measured by liquid chromatography-tandem mass spectrometry (LC-MS) to obtain a drug concentration-time curve.

The main pharmacokinetic parameters are shown in Table 1 below:

TABLE 1

| Parameter | Example 1 |
|---|---|
| Dose (mg/kg) | 1.0 |
| Cₘₐₓ (ng/mL) | 364 |
| AUC₀₋ₜ (ng · h/mL) | 446 |
| T_{1/2} (h) | 2.99 |

Intragastric administration: Twelve healthy male Han-Wister rats having a body weight of 250-350 g were provided by Beijing Vital River Laboratory Animal Technology Co., Ltd. and randomly divided into 4 groups. The above compounds of Example 1, Example 2, Example 3 and Example 4 were administered intragastrically at the doses (calculated based on free base) listed in Table 2 below, respectively. Before administration and 15 min, 0.5 h, 1.0 h, 2.0 h, 4.0 h, 8.0 h, 12 h, and 24 h after administration, jugular vein puncture was performed to take 0.05 mL of blood, plasma was separated and prepared, and the drug concentration in the plasma was measured by liquid chromatography-tandem mass spectrometry (LC-MS) to obtain a drug concentration-time curve.

The main pharmacokinetic parameters are shown in Table 2 below:

TABLE 2

| Parameter | Example 1 | Example 2 | Example 3 | Example 4 |
|---|---|---|---|---|
| Dose (mg/kg) | 80 | 100 | 93.2 | 81.1 |
| Cₘₐₓ (ng/mL) | 583 | 1024 | 1020 | 1376 |
| AUC₀₋ₜ (ng · h/mL) | 3968 | 13060 | 11213 | 9703 |
| T_{1/2} (h) | 3.36 | 6.65 | 6.04 | 6.17 |
| F (%) | 11.1% | 29.3% | 27.0% | 26.8% |

Conclusion: The bioavailability of the crystalline form II-A of the mesylate salt of the compound of formula (I), the crystalline form III-A of the hydrochloride salt of the compound of formula (I) and the crystalline form IV-C of the maleate salt of the compound of formula (I) is significantly better than that of the compound of formula (I) of Example 1.

### Test Example 2: Solubility Test

The dynamic solubility of the compound of formula (I) of Example 1, the crystalline form II-A of the mesylate salt of the compound of formula (I), the crystalline form III-A of the hydrochloride salt of the compound of formula (I) and the crystalline form IV-C of the maleate salt of the compound of formula (I) in water, SGF, FaSSIF and FeSSIF (taken at the time of 0.5 h, 1 h, 2 h, 4 h, and 24 h) was investigated.

Determination method: According to a feeding concentration of 10 mg/mL, about 50 mg of the sample were weighed and added to 5 mL of the corresponding medium, and the sample was dissolved by rotating at −25 rpm at room temperature. After rotating for 0.5 h, 1 h, 2 h, 4 h, and 24 h, respectively, 1 mL of the sample was taken and centrifuged at 12,000 rpm for 2 min at room temperature, and the supernatant was used for solubility determination.

The solubility results are shown in Table 3 below, in mg/mL.

TABLE 3

| Solvent | Sample | 0.5 h | 1 h | 2 h | 4 h | 24 h |
|---|---|---|---|---|---|---|
| water | crystaline form II-A | >9.2 | >9.2 | >9.4 | >9.8 | >9.1 |
| | of mesylate salt | | | | | |
| | crystaline form III-A | 8.2 | 8.8 | 9.7 | 9.0 | 9.7 |
| | of hydrochloride salt | | | | | |
| | crystaline form IV-C | 4.0 | 4.3 | 4.4 | 4.7 | 4.4 |
| | of maleate salt | | | | | |
| | compound of formula (I) | <1.0 | <1.0 | <1.0 | <1.0 | <1.0 |
| SGF | crystaline form II-A | >10.0 | >10.0 | >10.0 | >10.0 | >10.0 |
| | of mesylate salt | | | | | |
| | crystaline form III-A | >10.0 | >10.0 | >10.0 | >10.0 | >10.0 |
| | of hydrochloride salt | | | | | |
| | crystaline form IV-C | >10.0 | >10.0 | >10.0 | >10.0 | >10.0 |
| | of maleate salt | | | | | |
| | compound of formula (I) | <1.0 | <1.0 | <1.0 | <1.0 | <1.0 |
| FaSSIF | crystaline form II-A | 6.6 | 6.2 | 7.0 | 6.9 | 9.9 |
| | of mesylate salt | | | | | |
| | crystaline form III-A | 5.3 | 5.2 | 5.0 | 5.0 | 8.2 |
| | of hydrochloride salt | | | | | |
| | crystaline form IV-C | 6.1 | 6.2 | 7.2 | 7.7 | >9.9 |
| | of maleate salt | | | | | |
| | compound of formula (I) | <1.0 | <1.0 | <1.0 | <1.0 | <1.0 |
| FeSSIF | crystaline form II-A | 8.1 | >9.1 | >10.0 | >10.0 | >10.0 |
| | of mesylate salt | | | | | |
| | crystaline form III-A | >10.0 | >10.0 | >10.0 | >10.0 | >10.0 |
| | of hydrochloride salt | | | | | |
| | crystaline form IV-C | >10.0 | >10.0 | >10.0 | >10.0 | >10.0 |
| | of maleate salt | | | | | |
| | compound of formula (I) | <1.0 | <1.0 | <1.0 | <1.0 | <1.0 |

Conclusion: The crystalline form II-A of the mesylate salt of the compound of formula (I), the crystalline form III-A of the hydrochloride salt of the compound of formula (I) and the crystalline form IV-C of the maleate salt of the compound of formula (I) have higher dynamic solubility (>4.0 mg/mL) in water, SGF, FaSSIF and FeSSIF, which is significantly better than the dynamic solubility of the compound of formula (I) (free base) in the corresponding medium.

Since the mesylate salt of the compound of formula (I) achieves surprising and unexpected technical effects in both salt formation and crystallization, it is more suitable for further drug development.

Although the invention has been described in detail by the detailed description and examples for clarity, these description and examples should not be construed as limiting the scope of the present invention.

1. A crystalline form of a mesylate salt of a compound of formula (I):

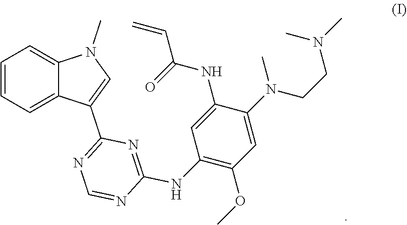 [SMILES: C=CC(=O)Nc1cc(Nc2ncnc(-c3cn(C)c4ccccc34)n2)c(OC)cc1N(C)CCN(C)C]

2. The crystalline form of the mesylate salt of the compound of formula (I) according to claim 1, which is crystalline form II-A, characterized in that the crystalline form II-A at least has an X-ray powder diffraction pattern having characteristic peaks expressed in 2θ° of 15.12°±0.2°, 22.28°±0.2° and 25.82°±0.2°.

3. A crystalline form of the hydrochloride salt of a compound of formula (I):

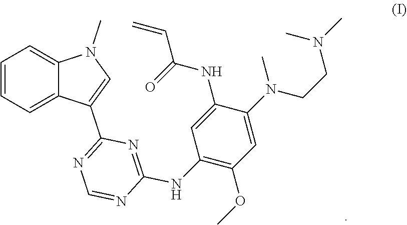 [SMILES: C=CC(=O)Nc1cc(Nc2ncnc(-c3cn(C)c4ccccc34)n2)c(OC)cc1N(C)CCN(C)C]

4. The crystalline form of the hydrochloride salt of the compound of formula (I) according to claim 3, which is crystalline form III-A, characterized in that the crystalline form III-A at least has an X-ray powder diffraction pattern having characteristic peaks expressed in 2θ° of 24.85°±0.2°, 14.08°±0.2° and 14.45°±0.2°.

5. A crystalline form of the maleate salt of a compound of formula (I):

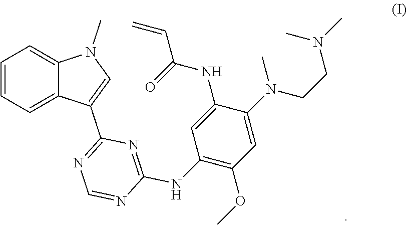 [SMILES: C=CC(=O)Nc1cc(Nc2ncnc(-c3cn(C)c4ccccc34)n2)c(OC)cc1N(C)CCN(C)C]

6. The crystalline form of the maleate salt of the compound of formula (I) according to claim 5, which is crystalline form IV-C, characterized in that the crystalline form IV-C at least has an X-ray powder diffraction pattern having characteristic peaks expressed in 2θ° of 20.71°±0.2°, 25.76°±0.2° and 17.00°±0.2″.

7. A pharmaceutical composition comprising the crystalline form according to claim 1 and a pharmaceutically acceptable carrier.

8. A method of treating non-small cell lung cancer (NSCLC) with EGFR exon 20 insertion mutation, comprising administering the crystalline form according to claim 1 to a patient.

9-10. (canceled)

11. A method of treating non-small cell lung cancer (NSCLC) with EGFR exon 20 insertion mutation, comprising administering the pharmaceutical composition according to claim 7 to a patient.
